(19) United States
(12) Patent Application Publication
(10) Pub. No.: US 2025/0161532 A1
(43) Pub. Date: May 22, 2025

(54) SELECTIVELY PERMEABLE ANNULUS FIBERS AND METHODS OF USING SAME

(71) Applicant: ASPECT BIOSYSTEMS LTD., Vancouver (CA)

(72) Inventors: Sam WADSWORTH, Vancouver (CA); Tamer MOHAMED, Vancouver (CA); Spiro GETSIOS, Nanaimo (CA); Valerio RUSSO, Vancouver (CA); Sheng PAN, Richmond (CA); Simon BEYER, Richmond (CA); James PIRET, Vancouver (CA); René Gonzalo PEDROZA LOZANO, Vancouver (CA)

(21) Appl. No.: 18/563,872

(22) PCT Filed: May 24, 2022
(86) PCT No.: PCT/CA22/50825

§ 371 Date: Nov. 22, 2023

Related U.S. Application Data
(60) Provisional application No. 63/192,552, filed on May 24, 2021.

(51) Int. Cl. A61L 27/26; A61L 27/20; A61L 27/38; A61L 27/52; A61L 27/54

(52) U.S. Cl. CPC A61L 27/26; A61L 27/20; A61L 27/38; A61L 27/52; A61L 27/54; A61L 2300/412; A61L 2400/18; A61L 2420/00

(57) ABSTRACT

Aspects of the invention include systems and methods for producing selectively permeable cell laden solid core fiber structures, and for producing three-dimensional (3D) biological structures for digital files. In embodiments, the printed fibers comprise living cells embedded in at least one annulus layer or the printed fibers comprise a solid core of at least one biological material. The tissue fiber can be used in tissue engineering as synthetic tissue structures.

### CROSS-REFERENCE TO RELATED APPLICATIONS

This application claims the benefit of priority to U.S. Provisional Application No. 63/192,552 filed on May 24, 2021.

### FIELD OF THE INVENTION

The present invention relates to systems and methods for producing selectively permeable and immunoprotective solid core fiber structures, and to three-dimensional (3D) printing of such structures from digital files. In some embodiments, the printed fibers comprise living cells.

### BACKGROUND OF THE INVENTION

The tissue engineering art has long sought to fabricate viable synthetic structures capable of mimicking and/or replacing living organs and tissues using myriad materials and methods. Cell encapsulation in particular has been repeatedly explored as a potential alternative to direct tissue transplantation and consequent life-long immunosuppression, but a number of challenges still remain. The essence of this approach is to place a semipermeable membrane around a therapeutic cell population (e.g., islet cells) that can effectively exclude immune cells, antibodies and complement components while ensuring sufficient diffusion of small molecules (e.g., glucose, oxygen) and desired biologically active agent (e.g., secretory peptides/proteins). De Vos et al., Adv Drug Deliv Rev. 2014; 67-68:15-34. Importantly, however, while glucose and oxygen are consumed at roughly the same molar rates, the latter is approximately 100 times less abundant physiologically. Colton, Adv Drug Deliv Rev. 2014; 67-68:93-110. Accordingly, the adequate passage of oxygen has consistently been one of the primary constraints in the effective clinical design of cell encapsulation devices.

A variety of device geometries have been explored, including microencapsulation in spheres and macroencapsulation in planar diffusion chambers. While microencapsulation in hydrogel beads provides a higher surface area to volume ratio for oxygen transport, settling and stacking of the beads has been reported after implantation to result in oxygen deprivation. Vaithilingam et al. Rev Diabet Stud. 2017; 14(1):51-78. Conversely, despite showing some promise in small animal studies, planar diffusion chambers have also suffered from considerable challenges in the scale up to human patients due to their intrinsically low surface area for mass transfer. Calafiore and Basta, Adv Drug Deliv Rev. (2014); 67-68: 84-92. A general limitation of all these approaches is the lack of control over the placement of the cells within the cell containing portion of the encapsulation device and its impact on oxygen supply. Accordingly, despite decades of research across the globe, an effective clinical application of cell encapsulation remains elusive (Orive et al., Trends Pharmacol Sci. (2015); 36:537-546).

Moreover, implantation into the body triggers an orchestrated biological response by both innate and adaptive immune systems against a device with the intention of eliminating it. The cellular response against perceived pathogens too large to be phagocytosed is mediated in part by macrophages that overexpress ECM proteins, such as fibronectin, and also produce pro-fibrogenic factors which enhance fibrogenesis by fibroblasts, resulting in the formation of a fibrotic capsule around the device. This fibrous capsule can interfere with device function particularly when they contain therapeutic cell populations which require access to nutrients and oxygen flow in order to perform their intended function. Although increasing the diameter of an implanted device can help reduce this foreign body response (FBR) and the consequent development of fibrosis, see, e.g., Watanabe et al., Biomaterials (2020), doi.org/10.1016/j.biomaterials.2020.120162, the requisite increase in size can also lead to oxygen deprivation and apoptosis for the cells in the device.

Accordingly, improvements in both design and materials are still needed to accommodate the opposing objectives of immune protection and nutrient passage, and to help mitigate the FBR response, and to enable consistent fiber-to-fiber deposition for synthetic structure manufacture. As such, there is a need for synthetic tissue structures that effectively balance capabilities that reduce or avoid immune system recognition and/or breach of such synthetic tissue structures with capabilities that ensure adequate passage of oxygen and nutrients to cells of the synthetic structure and without compromising their secretory capacity.

All prior art references listed herein are incorporated by reference in their entirety.

### SUMMARY OF INVENTION

The present invention successfully resolves the foregoing multiple conflicting objectives in the art with a selectively permeable bioprinted fiber comprising a solid core and one or more shell layers surrounding the solid core, wherein a biological material, e.g. living cells producing and secreting a biologically active agent of interest, are radially displaced outside of the solid core. In embodiments, the fiber comprises at least one annulus layer printed between the solid core and at least one external shell layer, wherein the cells are embedded within the at least one annulus layer, and preferably wherein the cells are homogeneously dispersed throughout the at least one annulus layer. In some embodiments, the cells are segmented/compartmentalized along a length of the bioprinted fiber, and/or the at least one external shell layer is immunoprotective and/or pro-vasculogenic. As demonstrated herein, radially displacing the embedded cell population in at least one annulus layer outside of the solid core improves both cell viability and function, increases secretory capacity and enables higher cell loadings than with conventional core-shell fibers.

According to one aspect, a bioprinted selectively permeable fiber is provided comprising a solid core, at least one annulus layer surrounding said solid core comprising at least one biological material embedded within a biocompatible material, and at least one external shell layer surrounding said at least one annulus layer. In embodiments, the at least one external shell layer comprises a cross-linked hydrogel material. In embodiments, the at least one biological material is homogeneously dispersed throughout the at least one annulus layer. In embodiments, the biological material is segmented/compartmentalized along the length of the fiber. In embodiments, the at least one external shell layer of the bioprinted selectively permeable fiber is immunoprotective and/or pro-vasculogenic.

Preferably, the biological material comprises a cell population and/or a cell-derived extracellular vesicle population producing/secreting a biologically active agent of interest, e.g., a therapeutic protein or nucleic acid, or extracellular vesicle. In some examples, the cell population comprises cells from endocrine and exocrine glands selected from the group consisting of pancreas, liver, thyroid, parathyroid, pineal gland, pituitary gland, thymus, adrenal gland, ovary, testis, enteroendocrine cells, stem cells, stem cell-derived cells or cells engineered to secrete a biologically active agent of interest. In some examples, the cell population releases cell-derived extracellular vesicles in the form of exosomes containing a therapeutic protein or nucleic acid. In some examples, the biocompatible material is selected from alginate, functionalized alginate, collagen-1, basement membrane proteins, collagen-4, collagen-2, fibronectin, vitronectin, laminin, decellularized extracellular matrices, hyaluronic acid, polyethylene glycol (PEG), poly(ethylene glycol) diacrylate (PEGDA) and other functionalized PEG, fibrin, gelatin, gelatin methacryloyl (GEL-MA), silk, chitosan, cellulose, poly(oligo(ethylene glycol) methyl ether methacrylate) (POEGMA), self assembling peptide hydrogels, and combinations thereof. In embodiments, the functionalized alginate may comprise one or more of methacrylated alginate, alginate furan, alginate thiol, alginate maleimide, alginate tetrazine, alginate norbornene, alginate hydrazide, RGD-alginate (arginine-guanidine-aspartate), YIGSR-alginate (Tyr-Ile-Gly-Ser-Arg), and covalent click alginates (e.g., alginate blended with 2-(Methacryloyloxy)ethyl]dimethyl-(3-sulfopropyl)ammonium hydroxide (DMAPS)-Ald and/or DMAPS-Hzd).

In some embodiments, the bioprinted selectively permeable fiber is reinforced. For example, the solid core may provide reinforcement to an overall fiber (e.g., a core-shell or annulus fiber of the present disclosure). As a representative example, a reinforced fiber may be comprised of a core material selected to have a higher tensile strength and/or modulus than the annulus layer and/or one or more external shell layers. In some embodiments, said solid core comprises a non-biodegradable material. In some examples, the solid core comprises a polymeric material selected from the group comprising or consisting of PEGDA and other functionalized PEG, POEGMA, polyvinyl alcohol (PVA) acrylamide, GEL-MA, or functionalized alginate, for example a functionalized alginate capable of covalent cross-linking. The functionalized alginate may comprise one or more of methacrylated alginate, alginate furan, alginate thiol, alginate maleimide, alginate tetrazine, alginate norbornene, alginate hydrazide, and covalent click alginates (e.g., alginate blended with DMAPS-Ald and/or DMAPS-Hzd).

In some embodiments, the at least one external shell layer of the bioprinted selectively permeable fiber is immunoprotective. In some examples, the external shell layer comprises an immunoprotective hydrogel material selected from alginate, chitosan, GEL-MA, PEG, PEGDA and other functionalized PEG (multi-arm PEG acrylate such as 4-arm PEG-tetra-acrylate (PEGTA) and PEFOA, and other PEG-based materials), POEGMA, poly-L-lysine (PLL), methacrylated hyaluronic acid (HA), thiolated hyaluronic acid, triazole, and combinations thereof. In some examples, the immunoprotective hydrogel material comprises a functionalized alginate. The functionalized alginate may comprise one or more of methacrylated alginate, alginate furan, alginate thiol, alginate maleimide, alginate hydrazide, alginate tetrazine, alginate norbornene, and covalent click alginates (e.g., alginate blended with DMAPS-Ald and/or DMAPS-Hzd). In some examples, the immunoprotective hydrogel material comprises or further comprises an acrylated zwitterionic monomer sulfobetaine methacrylate (SBMA) and/or PEGDA. In some examples, the immunoprotective hydrogel material comprises or further comprises a functionalized hyaluronic acid (for example thiolated HA) with a functionalized alginate or PEG (for example alginate-maleimide or PEG-maleimide).

In some embodiments, the at least one external shell layer of the bioprinted selectively permeable fiber is pro-vasculogenic. In some embodiments, the at least one pro-vasculogenic layer is positioned external to at least one external shell layer comprising immunoprotective materials. In some embodiments, the at least one external shell layer may be comprised of one or more pro-vasculogenic materials in addition to one or more immunoprotective materials. In some embodiments, a fiber of the present disclosure may comprise at least one external shell layer comprised of pro-vasculogenic materials, but may lack an immunoprotective shell layer. Pro-vasculogenic materials may comprise biodegradable materials including but not limited to fibrin, collagen, gelatin, GEL-MA, decellularized extracellular matrix (dECM), polymethacrylic acid, RGD-alginate, YIGSR-alginate, halyuronic acid, and the like.

In some embodiments, the diameter of the bioprinted selectively permeable fiber is between about 2.0 mm to 0.25 mm, more preferably between about 0.7 mm and about 1.7 mm, more preferably between about 1.5 mm to 0.45 mm, and most preferably between about 1.1 mm to 0.7 mm.

In some embodiments, the diameter of the solid core of the bioprinted selectively permeable fiber is between about 1.92 mm to 0.05 mm more preferably between about 1.1 mm to 0.41 mm, or between about 0.030 mm and about 1.0 mm, and most preferably between about 0.5 mm to 0.1 mm, for example about 0.3 mm.

In some embodiments, the thickness of the external shell layer of the bioprinted selectively permeable fiber is between about 0.01 mm to 0.3 mm, or between about 0.015 mm and about 0.5 mm, more preferably between about 0.025 mm to 0.2 mm, or between about 0.05 mm to 0.125 mm. In embodiments, the thickness is about 0.150 mm.

In some embodiments, the thickness of the annulus layer of the bioprinted selectively permeable fiber is between about 0.01 mm to 0.4 mm, more preferably between about 0.025 mm to 0.3 mm, more preferably between about 0.1 mm to about 0.3 mm, and most preferably between about 0.05 mm to 0.2 mm. In embodiments, the thickness is about 0.2 mm.

In another aspect, methods for bioprinting a selectively permeable annulus fiber are provided comprising dispensing a core material through a core channel, a first annulus layer material and optionally a second annulus layer material through a first shell channel, and a cross-linkable material through a second shell channel via a coaxial microfluidic device or multi-shell print head, wherein one of the first or second annulus layer materials comprises the biological material, preferably in a biocompatible material, optionally wherein the annulus layer materials are alternately dispensed to create segments/compartments of biological material along the length of the fiber. In some embodiments, the first annulus layer material and the second annulus layer material comprise the same biocompatible material.

According to another aspect, a bioprinted selectively permeable tissue fiber is provided comprising a solid core comprising at least one biological material segmented/compartmentalized in a biocompatible material along the length of the fiber, and at least one external shell layer surrounding said solid core comprising a cross-linked hydrogel material. Preferably, the biological material comprises a cell population producing/secreting a biologically active agent of interest, e.g. a therapeutic protein or nucleic acid, or a cellular vesicle comprising same. In some examples, the cell population comprises cells from endocrine and exocrine glands selected from the group consisting of pancreas, liver, thyroid, parathyroid, pineal gland, pituitary gland, thymus, adrenal gland, ovary, testis, enteroendocrine cells, stem cells, stem cell-derived cells or cells engineered to secrete a biologically active agent of interest. In some examples, the cell population releases cell-derived extracellular vesicles in the form of exosomes containing a therapeutic protein or nucleic acid. In some examples, the biocompatible material is selected from alginate, functionalized alginate, collagen-1, basement membrane proteins, collagen-4, collagen-2, fibronectin, vitronectin, laminin, decellularized extracellular matrices, hyaluronic acid, PEG, PEGDA and other functionalized PEG, fibrin, gelatin, GEL-MA, silk, chitosan, cellulose, POEGMA, self assembling peptide hydrogels, and combinations thereof. In embodiments, the functionalized alginate may comprise one or more of methacrylated alginate, alginate furan, alginate thiol, alginate maleimide, alginate tetrazine, alginate norbornene, alginate hydrazide, RGD-alginate (arginine-guanidine-aspartate), YIGSR-alginate (Tyr-Ile-Gly-Ser-Arg), and covalent click alginates (e.g., alginate blended with DMAPS-Ald and/or DMAPS-Hzd).

In some embodiments, the solid core further comprises a cross-linker for cross-linking the external shell layer from the inside-out. For example, the solid core may comprise a cross linker capable of cross-linking external shell material, whereas material corresponding to the solid core may be cross linked by a different mechanism. As a representative example, the solid core may include PEGDA+calcium+a photoinitiator, whereas the external shell may comprise alginate. The calcium ions in the core may cross link the alginate-based external shell from the inside out. Illumination of the fiber may cause activation of the photoinitiator and thus cross-linking of the core.

In some embodiments, the external shell layer of the bioprinted selectively permeable fiber is immunoprotective and/or pro-vasculogenic. In some examples, the bioprinted selectively permeable fiber has an immunoprotective shell comprised of immunoprotective material(s) that is surrounded in turn by a pro-vasculogenic shell comprised of pro-vasculogenic material(s). In some examples, the immunoprotective material is selected from alginate, chitosan, GEL-MA, PEG, PEGDA and other functionalized PEG (multi-arm PEG acrylate such as PEGTA and PEFOA, and other PEG-based materials), POEGMA, PLL, triazole, and combinations thereof. In some examples, the immunoprotective hydrogel material comprises a functionalized alginate. The functionalized alginate may comprise one or more of methacrylated alginate, alginate furan, alginate tetrazine, alginate norbornene, alginate thiol, alginate maleimide, alginate hydrazide, and covalent click alginates (e.g., alginate blended with DMAPS-Ald and/or DMAPS-Hzd). In some examples, the immunoprotective hydrogel material comprises or further comprises an acrylated zwitterionic monomer (SBMA) and/or PEGDA. In some examples, the immunoprotective hydrogel material comprises or further comprises a functionalized hyaluronic acid (for example thiolated HA) with a functionalized alginate or PEG (for example alginate-maleimide or PEG-maleimide). In some examples, the pro-vasculogenic material is selected from fibrin, collagen, gelatin, GEL-MA, decellularized extracellular matrix (dECM), polymethacrylic acid, RGD-alginate, YIGSR-alginate, halyuronic acid, and the like.

In some embodiments, the diameter of the bioprinted selectively permeable fiber is between about 2.0 mm to 0.2 mm, more preferably between about 1.5 mm to 0.5 mm, and most preferably between about 1.1 mm to 0.8 mm.

In some embodiments, the diameter of the solid core of the bioprinted selectively permeable fiber is between about 1.98 mm to 0.18 mm more preferably between about 1.48 mm to 0.48 mm, and most preferably between about 0.9 mm to 0.50 mm.

In some embodiments, the thickness of the external shell layer of the bioprinted selectively permeable fiber is between about 0.01 mm to 0.3 mm, more preferably between about 0.025 mm to 0.2 mm, and most preferably between about 0.05 mm to 0.15 mm.

In another aspect, methods for bioprinting a selectively permeable fiber comprising a solid core comprising at least one biological material segmented/compartmentalized along the length of the fiber are provided. In one embodiment, the method comprises discontinuously dispensing a biocompatible material comprising the biological material through a core channel of a coaxial microfluidic device or multi-shell print head, while continually dispensing an immunoprotective cross-linkable material through at least one shell channel, wherein the shell material fills in the fiber between segments of the core. In another embodiment, the method comprises alternating between dispensing a first core material and a second core material through a core channel of a coaxial microfluidic device or multi-shell print head, while continually dispensing an immunoprotective cross-linkable material through at least one shell channel, wherein one of the first or second core materials comprises the biological material, preferably in a biocompatible material, such that the alternating core materials create segments/compartments of biological material along the length of the fiber. In some embodiments, the first core material and the second core material comprise the same biocompatible material. In embodiments, the at least one external shell layer of the bioprinted selectively permeable fiber is immunoprotective and/or pro-vasculogenic.

In some embodiments, the biological material comprises a cell population producing/secreting a biologically active agent of interest, e.g., a therapeutic protein/peptide, nucleic acid, or extracellular vesicle. In some examples, the cell population comprises cells from endocrine and exocrine glands selected from the group consisting of pancreas, liver, thyroid, parathyroid, pineal gland, pituitary gland, thymus, adrenal gland, ovary, testis, enteroendocrine cells, stem cells, stem cell-derived cells or cells engineered to secrete a biologically active agent of interest. In some examples, the cell population releases cell-derived extracellular vesicles in the form of exosomes containing a therapeutic protein or nucleic acid. In some examples, the biocompatible material is selected from alginate, collagen-1, basement membrane proteins, collagen-4, collagen-2, fibronectin, vitronectin, laminin, decellularized extracellular matrices, hyaluronic acid, PEG, PEGDA and other functionalized PEG, fibrin, gelatin, GEL-MA, silk, chitosan, cellulose, POEGMA, self assembling peptide hydrogels, and/or functionalized alginate. Examples of functionalized alginates include but are not limited to methacrylated alginate, alginate furan, alginate thiol, alginate maleimide, alginate tetrazine, alginate norbornene, alginate hydrazide, RGD-alginate, YIGSR-alginate, and covalent click alginates (e.g., alginate blended with DMAPS-Ald and/or DMAPS-Hzd).

In some embodiments, the at least one external shell layer of the bioprinted selectively permeable fiber is immunoprotective and/or pro-vasculogenic. In some embodiments, an external shell layer comprising immunoprotective materials is in turn surrounded by an external shell layer comprising pro-vasculogenic materials. In some examples, the immunoprotective material is selected from alginate, chitosan, GEL-MA, PEG, PEGDA and other functionalized PEG (multi-arm PEG acrylate such as PEGTA and PEFOA, and other PEG-based materials), POEGMA, PLL, triazole, and combinations thereof. In some examples, the immunoprotective hydrogel material comprises a functionalized alginate. The functionalized alginate may comprise one or more of methacrylated alginate, alginate furan, alginate tetrazine, alginate norbornene, alginate thiol, alginate maleimide, alginate hydrazide, and covalent click alginates (e.g., alginate blended with DMAPS-Ald and/or DMAPS-Hzd). In some examples, the immunoprotective hydrogel material comprises or further comprises an acrylated zwitterionic monomer (SBMA) and/or PEGDA. In some examples, the immunoprotective hydrogel material comprises or further comprises a functionalized hyaluronic acid (for example thiolated HA) with a functionalized alginate or PEG (for example alginate-maleimide or PEG-maleimide). In some examples, the pro-vasculogenic material is selected from fibrin, collagen, gelatin, GEL-MA, decellularized extracellular matrix (dECM), polymethacrylic acid, RGD-alginate, YIGSR-alginate, halyuronic acid, and the like.

In some embodiments, the method further comprises dispensing one or more cross-linking agents through the core channel contemporaneously with the biocompatible materials so as to cross-link the selectively permeable fiber from inside-out. In some embodiments, the method for producing the selectively permeable tissue fiber further comprises dispensing one or more sheath fluids comprising one or more cross-linking materials through a sheath channel so as to cross-link the selectively permeable fiber from the outside-in.

In some embodiments, the selectively permeable tissue fiber is of a diameter of between about 2.0 mm to 0.2 mm, more preferably between about 1.5 mm to 0.5 mm, and most preferably between about 1.1 mm to 0.8 mm.

In some embodiments, the diameter of said solid core is between about 1.98 mm to 0.18 mm, more preferably between about 1.48 mm to 0.48 mm, and most preferably between about 0.9 mm to 0.5 mm.

In some embodiments, the thickness of said immunoprotective external shell layer is between about 0.01 mm to 0.3 mm, more preferably between about 0.025 mm to 0.2 mm, and most preferably between about 0.05 mm to 0.15 mm.

In another aspect, methods for producing/secreting a biological agent of interest are provided, comprising culturing one or more of the aforementioned bioprinted selectively permeable fibers in vitro or in vivo, including in a patient in need thereof, wherein the selectively permeable fibers comprise a biological material capable of producing/secreting the biologically active agent of interest segmented/compartmentalized along a length of the bioprinted fiber, and/or wherein the external shell layer of said fiber(s) is immunoprotective.

### BRIEF DESCRIPTION OF THE DRAWINGS

FIG. 1 depicts a reaction scheme for the methacrylation of alginate.

FIG. 2 depicts chemical structures of a zwitterionic monomer (e.g., sulfobetaine methacrylate (SBMA)) and poly(ethylene glycol) diacrylate (PEGDA), and illustrates that photo cross-linking of SBMA with PEGDA results in a network of cross-linked hydrogel with pendant zwitterionic groups.

FIG. 3 depicts exemplary fiber architectures and cross-linking strategy leading to fiber-fiber adhesion, according to embodiments.

FIG. 4 depicts chemical structures of zwitterionic polymers that can cross-link hydrogel materials via click chemistry, according to embodiments.

FIGS. 5A-5F depicts fiber geometries for the disclosed analytical oxygen transport model. Shown are representative examples of core-shell (FIG. 5A) and core-annulus-shell (FIG. 5D) fibers. The geometry of these alginate fibers, containing a multiplicity of cells (i.e. islets) of different sizes in a confined fiber volume, was simplified to a cell region where the islet volume is homogenously distributed (FIGS. 5B, 5E) with an average cell volume fraction E (FIGS. 5C, 5F).

FIGS. 6A-6D depicts fiber cross-sections for the disclosed oxygen transport model. Shown are schematic cross-sections of core-shell (FIGS. 6A, 6C) and core-annulus-shell fibers (FIGS. 6B, 6D) shown in the absence (FIGS. 6A, 6B) and presence (FIGS. 6C, 6D) of oxygen limitations. Encapsulant material shown in green; oxygenated and anoxic cell layers shown in red and gray, respectively. Fiber dimensions denoted by R₁, R₂ and R₃, correspond to radii of fibers, cell regions and acellular cores. Oxygen partial pressures surrounding fiber and at which cells die indicated by P_{E} and P_{D}. In the presence of oxygen limitations, the radial boundary of the anoxic cell layer (r_{A}) was calculated iteratively.

FIGS. 7A-7D depict fiber cross-sections of the disclosed insulin secretion rate model. Shown are cross-sections of core-shell (FIGS. 7A, 7C) and core-annulus-shell fibers (FIGS. 7B, 7D) shown in the absence (FIGS. 7A, 7B) and presence (FIGS. 7C, 7D) of oxygen limitations. Encapsulant material shown in green; cell layers with maximum and submaximal insulin secretion rate shown in red and orange, respectively; anoxic cell layer shown in gray. Fiber dimensions denoted by R₁, R₂ and R₃ correspond to radii of fibers, cell regions and acellular cores. Oxygen partial pressures surrounding fibers, required for maximum insulin secretion rate and at which cells die indicated by, P_{E}, P_{I} and P_{D}. Radial boundaries of cell layer with submaximal insulin secretion rate (r_{I}) and anoxic cell layer (r_{A}) were calculated iteratively where applicable.

FIGS. 8A-8F depict fiber cross-sections of fully oxygenated and maximum insulin secretion rate scenarios. Shown are cross-sections of core-shell (FIGS. 8A, 8C, 8E) and core-annulus-shell fibers (FIGS. 8B, 8D, 8F) with cell volume fraction E adjusted to attain cell regions with maximum insulin secretion rate (FIGS. 8A, 8B), or fully oxygenated (FIGS. 8C, 8D). Increasing cell volume fraction leads beyond the maximum E for a fully oxygenated cell region leads to formation of anoxic cell layers (FIGS. 8E, 8F). Encapsulant material shown in green; cell layers with maximum and submaximal insulin secretion rate shown in red and orange, respectively; anoxic cell layer shown in gray. Fiber dimensions denoted by R₁, R₂ and R₃ correspond to radii of fibers, cell regions and acellular cores. Oxygen partial pressures surrounding fibers, required for maximum insulin secretion rate and at which cells die indicated by, P_{E}, P_{I} and P_{D}. Radial boundaries of cell layer with submaximal insulin secretion rate (r₁) and anoxic cell layer (r_{A}) were calculated iteratively where applicable.

FIGS. 9A-9E depict theoretical oxygenation of 1 mm fibers with 150 μm immunoprotective shells exposed to 40 mm Hg. Equations 7 to 9 (refer to Example 1 below), were solved to calculate the oxygen profiles for core-shell (FIGS. 9A, 9C, 9E) and core-annulus-shell (FIGS. 9B, 9D) fibers with various levels of cell volume fraction E. Fibers with core-shell geometry had 350-μm radii cell region cores; core-annulus shell fibers had 150-μm acellular cores, surrounded by cell regions 200 μm in thickness. In these panels, orange dashed lines denote presence of cell layers with submaximal insulin secretion rate.

FIGS. 10A-10C depict theoretical insulin secretion rate of 1 mm fibers with 150 μm immunoprotective shells exposed to 40 mm Hg. Cell loadings for each of the fibers (FIG. 10A). Secretory capacities for the same fibers (FIG. 10B), and for core-annulus-shell fibers with various cell region thicknesses and cell volume fraction E adjusted to obtain maximum insulin secretion rate (FIG. 10C). Oxygen profiles were used in equations 11 to 16 (refer to Example 1 below) to estimate insulin secretion rates in fiber cross-sections.

FIG. 11 depicts a key for use in transforming results from sensitivity analyses to fiber cross-sections, and is relevant to use in conjunction with FIGS. 12A-13D.

FIGS. 12A-12E illustrate sensitivity analysis of parameters used in oxygen transport and insulin secretion rate models. Oxygen profiles resulting cell layer radial boundaries were obtained for the 1 mm core-annulus-shell fiber with cell volume fraction ε=22.4% (dashed line in all panels) while varying model parameters values retrieved from the literature and shown in Table 2. Encapsulant material shown in green; cell layers with maximum and submaximal insulin secretion rate shown in red and orange, respectively; anoxic cell layer shown in gray. Effects of increase/decrease in values of oxygen partial pressures at implant surface P_{E} (FIG. 12A), required for maximum insulin secretion rate P_{I} (FIG. 12B), and at which cells die P_{D} (FIG. 12C), maximum oxygen consumption rate of cells Vₘₐₓ (FIG. 12D), diffusion coefficients of oxygen in cells (αD)_{cells} (FIG. 12E) and in alginate hydrogel, mean of (αD)₁ and (αD)_{c,2} (FIG. 12F).

FIGS. 13A-13D illustrate sensitivity analysis of fiber parameters. Oxygen profiles and resulting cell layer radial boundaries were obtained for the 1-mm core-annulus-shell fiber with cell volume fraction ε=22.4% (dashed line in all panels) while varying fiber parameters. Encapsulant material shown in green; cell layers with maximum and submaximal insulin secretion rate shown in red and orange, respectively; anoxic cell layer shown in gray. Effects of increase/decrease in cell volume fraction ε (FIG. 13A), shell thickness with constant R₂. (FIG. 13B), acellular core radius R₃ with constant annular area π(R₂²−R₃²) (FIG. 13C) and constant annular thickness R₂−R₃ (FIG. 13D).

FIGS. 14A-14B illustrate actual images and simplified geometries of solid core, single shell fibers used in analytical models of the present disclosure.

FIGS. 15A-15B illustrate different view of simplified geometries for annulus fibers of the present disclosure.

FIG. 16 is a table with various assumptions on model parameters used in some models of annulus fibers of the present disclosure.

FIG. 17A illustrates example output from an oxygen profiling model of devices comprised of spherical and cylindrical geometries.

FIG. 17B illustrates device dimensions used to obtain the output shown at FIG. 17A.

FIG. 18A illustrates example output from the oxygen profiling model for an annulus device of the present disclosure, overlaid with the output from the devices comprised of spherical and cylindrical geometries (FIG. 17A).

FIG. 18B illustrates device dimensions used to obtain the output from the oxygen profiling model for the annulus device as shown at FIG. 18A.

FIG. 19A illustrates example output from the oxygen profiling model for two other annulus devices of the present disclosure.

FIG. 19B illustrates device dimensions used to obtain the output from the oxygen profiling model for the annulus device as shown at FIG. 19A.

FIGS. 20A-20I illustrate modeled partial pressure of oxygen as a function of radial distance for various annulus devices (FIGS. 20A, 20D, 20G), and modeled local insulin secretion (% maximum) as a function of radial distance for the various annulus devices (FIGS. 20B, 20E, 20H). Parameters for the various devices are illustrated at FIG. 20C, FIG. 20F, and FIG. 20I.

FIGS. 21A-21C show modeled partial pressure of oxygen as a function of outer shell thickness at an annulus core interface (FIG. 21A), based on the general fiber structure of FIG. 21B for various devices having parameters as shown at FIG. 21C.

FIGS. 22A-22C illustrate scenarios for O₂ transfer and insulin secretion rate models of the present disclosure. Depicted are schematics of maximal insulin secretion rate (FIG. 22A), fully oxygenated core (FIG. 22B), and inner anoxic region (FIG. 22C).

FIGS. 23A-23B are graphs illustrating oxygenation and insulin secretion rate in core-shell fibers derived from analytical models discussed with regard to FIGS. 22A-22C. Shown are effects of volume cell fraction (FIG. 23A) and shell thickness (FIG. 23B) on the cross-sectional areas of the fully oxygenated layer (black) and the layer with maximal insulin secretion rate (red). The dashed line at FIG. 23B illustrates FITC-dextran fibers.

FIGS. 24A-24F illustrate data corresponding to FITC-dextran release from core-shell fibers. FIG. 24A illustrates FITC-dextran recovered from supernatants of four types of fibers (n=3). Depicted at FIGS. 24B-24E are actual release profiles for 4 kDa (FIG. 24B), 70 kDa (FIG. 24C), 100 kDa (FIG. 24D), and 250 kDa (FIG. 24E). FIG. 24F is a graph showing time for 90% release of total dextran as a function of molecule size.

FIGS. 25A-25B are graphs showing one factor at a time sensitivity analysis of model parameters discussed with regard to FIGS. 22A-23B. Shown are effectives of uncertainty in values of islet oxygen consumption rate (FIG. 25A) and effective diffusivity of oxygen in alginate hydrogel (FIG. 25B) on the oxygenated layer.

FIG. 26A is a graph showing tensile strength and percentage decrease in mechanical strength approximately one month post-print of Min6 clusters printed in C/S fibers, with varying ratios of zwitterionic alginate to SLG100.

FIGS. 26B-26E are brightfield images of the fibers used to obtain the data depicted at FIG. 26A.

FIGS. 27A-27D represent live/dead images of primary human islets (PHI) at 24 hours for core shell (C/S fibers) containing 1.5% SLG in the core and varying shell materials, specifically SLG 100 alginate (FIG. 27A), pure zwitterionic alginate (UBC) (FIG. 27B), pure zwitterionic alginate (in-house) (FIG. 27C), and DMAPS-Ald/hydrazide DMAPS-Hzd (FIG. 27D).

FIGS. 27E-27H represent live/dead images of PHI of the same fibers of FIGS. 27A-27D, at 7 days post print. Specifically, shell materials included SLG100 (FIG. 27E), pure zwitterionic alginate (UBC) (FIG. 27F), pure zwitterionic alginate (in-house) (FIG. 27G), and DMAPS-Ald/hydrazide DMAPS-Hzd (FIG. 27H).

FIGS. 28A-28C illustrate assessment of F-F adhesion of fibers comprised of a particular composition (FIG. 28A), cross-linked on-chip. FIG. 28B illustrates an image of the patterned structure tested, and FIG. 28C illustrates a high-resolution image of a fiber of the patterned structure of FIG. 28B.

FIGS. 28D-28F illustrate assessment of F-F adhesion of fibers comprised of a particular composition (FIG. 28D), cross-linked post-print. FIG. 28E illustrates an image of the patterned structure tested, and FIG. 28F illustrates a high-resolution image of fibers of the patterned structure of FIG. 28E.

FIGS. 28G-28I depict an illustration of an inside-out (I-O) cross linking methodology as disclosed herein (FIG. 28G), and comparison of fibers cross linked via outside-in (O-I) cross linking (FIG. 28H) with fibers cross linked via I-O cross linking methodology (FIG. 28I).

FIG. 29 depicts a graph showing basal c-peptide plasma levels from mice in which solid core, single shell fibers (C/S) were implanted into healthy C57BL/6 mice.

FIG. 30 are graphs showing blood glucose levels as a function of days post implantation (FIG. 30) of C/S fibers comprised of varying ratios of zwitterionic alginate comprising PHI cells into diabetic immune-competent mice.

FIGS. 31A-31B are graphs showing blood glucose levels as a function of time after intraperitoneal glucose tolerance test post-implantation of bioprinted C/S human islet fibers in immune-competent diabetic C57BL/6 mice.

FIGS. 32A-32B are graphs showing data from three representative immune-competent diabetic C57BL/6 mice implanted with bioprinted C/S human islet fibers pertaining to c-peptide levels after glucose injection.

FIGS. 33A-33C are images of live/dead staining of bioprinted C/S human islet fibers retrieved from three representative immune-competent diabetic C57BL/6 mice.

FIGS. 34A-34B depict graphs of glucose levels as a function of days post-implant of C/S human islet fibers in immune-deficient diabetic mice.

FIGS. 35A-35D depict graphs showing glucose levels as a function of time following glucose tolerance tests initiated at 36 and 39 days post-implant (FIGS. 35A-35B), and 82 and 85 days post-implant (FIGS. 35C-35D) of bioprinted C/S human islet fibers into immune-deficient diabetic mice.

FIGS. 36A-36B are graphs showing c-peptide measurements pre-implant, 72 days post-implant, and 90 days post-implant (post-OGTT) obtained from immune-deficient diabetic mice into which C/S human islet fibers were implanted.

FIG. 37 depicts live/dead staining conducted on fiber devices retrieved from the immune-deficient diabetic mice at 72 and 88 days post-implant.

FIG. 38 depicts a printed annulus fiber with the annulus layer containing cell aggregates of primary human islets.

FIGS. 39A-39B depict images of an annulus fiber with the annulus layer containing primary human hepatocytes (PHH). FIG. 39B includes reference circles to delineate the various layers.

FIGS. 40A-40D depict data showing viability and functionality of annulus fibers as compared to core shell fibers of the present disclosure. FIG. 40A shows images of life/dead staining for core shell and annulus fibers at various time points post printing. FIG. 40B is a graph showing alamar blue fluorescence intensity at various time points for core shell fibers versus annulus fibers. FIG. 40C is a graph showing albumin production (ng/million cells/day) for core shell fibers versus annulus fibers. FIG. 40D is a graph showing urea production (μg/million cells/day) for core shell fibers versus annulus fibers.

FIGS. 41A-41C depict examples of materials switching with respect to segmentation of the fibers of the present disclosure. FIG. 41A is an illustration of a core-shell fiber in one embodiment of the present disclosure, FIG. 41B depicts a fiber with two different shell materials, and FIG. 41C depicts a fiber with two different core materials.

### DETAILED DESCRIPTION

Cell encapsulation can potentially reduce or eliminate the need for life-long immunosuppression associated with conventional cell replacement therapies. Since encapsulated cells are oxygenated via passive diffusion and typically implanted into sites with relatively low oxygen concentrations, it is critical to ensure adequate oxygen diffusion in order to maintain cell viability and function. In the present invention, multi-layered cell-laden fibers are provided where the cell-containing layer is constricted between two acellular layers, i.e., radially displaced from a solid core into an annulus layer. Preferred geometries are provided based on oxygen transfer modeling in comparison with conventional core-shell fibers, and sensitivity analyses illustrate the effects of variations in model parameters and fiber dimensions on the oxygen profiles of encapsulated cells and their secretion rates. Remarkably, and as further demonstrated herein, the present invention also enables an increase in cell loading capacity without compromising the secretory capacity of and/or triggering anoxic conditions for the encapsulated cells.

Aspects of the invention include compositions comprising bioprinted and selectively permeable fibers having a solid, i.e., non-hollow core and one or more shell layers surrounding the core, wherein the fiber comprises at least one biological material capable of producing and/or secreting a biologically active agent of interest, e.g. a therapeutic protein or nucleic acid embedded in an annulus layer between the solid core and an external shell layer. In some embodiments, the biological material is segmented/compartmentalized along the length of the fiber. In some embodiments, the outermost shell of the fiber may be immunoprotective and/or pro-vasculogenic. In some embodiments, at least one immunoprotective layer surrounds the annulus layer, and at least one pro-vasculogenic layer surrounds said at least one immunoprotective layer.

In some embodiments, the core of the bioprinted fibers may comprise at least one biological material, and the core may be surrounded by a single shell layer. In such an example, the single shell layer may comprise an immunoprotective hydrogel material and/or a pro-vasculogenic material. In some embodiments, the core may comprise at least one biological material, and the core may be surrounded by an immunoprotective hydrogel layer, which is surrounded in turn by a pro-vasculogenic layer.

In some embodiments, the core of the bioprinted fibers may be free of biological material, and the core may be surrounded by a plurality of shell layers. In such an example, an internal layer comprising a cellular layer (i.e., annulus layer) may be sandwiched between, for example, the core and at least one external shell layer. In such an example, the at least one external shell layer may comprise an immunoprotective hydrogel material and/or a pro-vasculogenic material. In some embodiments, the annulus layer may be surrounded by an immunoprotective layer, which is surrounded in turn by a pro-vasculogenic layer. Optionally, in some embodiments, the core may comprise a non-material of a higher tensile strength and/or modulus than surrounding layers for reinforcement.

In additional embodiments described herein, the biocompatible material(s) may be segmented/compartmentalized along the length of the fiber so as to reduce the overall cell loss in the event of fiber breach (e.g. by immune effector cells) or other rupture. In some embodiments, the core of the bioprinted fiber comprises the at least one biological material capable of producing the biologically active agent(s) of interest. In some embodiments, the core is free of biological materials, and the fiber further comprises at least one internal annulus layer surrounding the core comprising the at least one biological material capable of producing the biologically active agent(s) of interest. For example, where the core comprises the biological material, the core may comprise at least one biological material that is segmented/compartmentalized along the length of the fiber. In examples, the core may be continuous, but the biological material may be segmented/compartmentalized. In embodiments comprising an annulus layer, the annulus layer may be continuous but the biological material therein may be segmented/compartmentalized.

### Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control.

As used herein when referring to a “cell”, “cell line”, “cell culture” or “cell population” or “population of cells”, the term “isolated” refers to being substantially separated from the source of the cells such that the living cell, cell line, cell culture, cell population or population of cells are capable of being cultured in vitro for extended periods of time. In addition, the term “isolating” can be used to refer to the physical selection of one or more cells out of a group of two or more cells, wherein the cells are selected based on cell morphology and/or the expression of various markers.

The term “displace” as used herein refers to the ability of a first material or fluid to remove a second material or fluid from a given position. For example, in some embodiments, a buffer solution is configured to displace an input material from a position within a dispensing channel (e.g., from a proximal end of the dispensing channel). In some embodiments, a displacement is an instantaneous displacement, which occurs in less than about one second, such as about 900, 800, 700, 600, 500, 400, 300, 200, or 100 milliseconds or less.

The term “solidified” as used herein refers to a solid or semi-solid state of material that maintains its shape fidelity and structural integrity upon deposition. The term “shape fidelity” as used herein means the ability of a material to maintain its three dimensional shape without significant spreading. In some embodiments, a solidified material is one having the ability to maintain its three dimensional shape for a period of time of about 30 seconds or more, such as about 1, 10 or 30 minutes or more, such as about 1, 10, 24, or 48 hours or more. The term “structural integrity” as used herein means the ability of a material to hold together under a load, including its own weight, while resisting breakage or bending.

In some embodiments, a solidified composition is one having an elastic modulus greater than about 5, 10, 15, 20 or 25 kilopascals (kPa), more preferably greater than about 30, 40, 50, 60, 70, 80 or 90 kPa, still more preferably greater than about 100, 110, 120 or 130 kPa. Preferred elastic modulus ranges include from about 5, 10, 15, 20, 25 or 50 Pa to about 80, 100, 120 or 140 kPa. According to the subject invention, the elastic modulus of an input material can be advantageously varied according to the intended function of the input material. In some embodiments, a lower elastic modulus is employed to support cell growth and migration, while in other embodiments, a much higher elastic modulus can be used. In some embodiments, the elastic modulus may vary between different layers within a fiber. In an exemplary embodiment, the core of the fiber may have a relatively high elastic modulus, the internal cellular layer may have a lower modulus than the core, the outer shell may be even lower still in order to reduce FBR.

The term “hydrogel” as used herein refers to a composition comprising water and a network or lattice of polymer chains that are hydrophilic.

The term “sheath fluid” or “sheath solution” as used herein refers to a fluid that is used, at least in part, to envelope or “sheath” a material as the material is passing through a fluid channel. In some embodiments, a sheath fluid comprises an aqueous solvent, e.g., water or glycerol. In some embodiments, a sheath fluid comprises a chemical cross-linking agent. Non-limiting examples of cross-linking agents include divalent cations (e.g. Ca²⁺, Ba²⁺, Sr²⁺, etc.), thrombin, and pH modifying chemicals, such as sodium bicarbonate.

The terms “segmented/compartmentalized” as used herein refer to a discontinuous nature of a biological material included in core or cellular layer of the fibers disclosed herein, e.g. wherein there are intentional gaps in the deposition of the biological material along a length of the fiber. The spacing (e.g., length) between such segments/compartments may be regular (e.g., an approximately same spacing between regions of biological material), or the spacing may be different. The regions of biological material may, in some embodiments, be approximately the same in terms of length, or may be different. Cell density in different segments/compartments along a length of a fiber may be the same or different between different segments/compartments.

The term “selectively permeable” as used herein refers to a nature of core and/or shell layers of fibers of the present disclosure to allow for passage of some molecules or ions while preventing the passage of other molecules or ions. In some embodiments, the selectively permeable nature allows passage of smaller molecular species to the exclusion of larger molecular species.

The term “solid core” as used herein refers to a core of a fiber of the present disclosure that is comprised of a particular material (e.g., hydrogel cross-linkable by a chemical cross-linking agent), such that the core does not comprise a lumen along the entire length of the fiber. The term is not intended to refer to a core that is entirely impenetrable along its length, as solid cores of the present disclosure may enable the passage of particular fluids, molecules and/or ionic species throughout the core.

The term “annulus fibers” as used herein refer to fibers that are comprised of a solid core, and one or more shell layers surrounding the solid core. In embodiments, the core of an annulus fiber is surrounded by a first inner shell, and by a second outer shell, although fibers with greater numbers of shells (e.g., three, four, five, or more) are included within the definition of an annulus fiber of the present disclosure. In embodiments where the core is surrounded by a first inner shell, and by a second outer shell, the first inner shell may comprise a biological material, for example a cell population, and the second outer shell may comprise an immunoprotective and/or pro-vasculogenic layer. In embodiments with greater than two shell layers surrounding the core, an annulus layer surrounding the core may comprise a biological material, an immunoprotective layer may surround the annulus layer, and a pro-vasculogenic layer may surround the immunoprotective layer. In embodiments, the biological material is segmented/compartmentalized along a length of the particular shell layer(s) comprising the biological material.

The term “biocompatible materials” as used herein refer to materials in which biological materials including but not limited to cells can be incorporated into and/or in contact with said biocompatible materials and where said biocompatible materials do not exhibit an adverse effect on the ability of the biological materials to carry out one or more functions (e.g., cellular functions including but not limited to secretion of biologically relevant molecular species, agonist/receptor binding, signal transduction, and the like). Examples of biocompatible materials as herein disclosed can include but are not limited to alginate, functionalized alginate (e.g. RGD-alginate, YIGSR-alginate), collagen, collagen-1, basement membrane proteins, collagen-4, collagen-2, fibronectin, fibrin, gelatin, vitronectin, laminin, decellularized extracellular matrices (dECM), hyaluronic acid (HA), polyethylene glycol (PEG), PEGDA and other functionalized PEG, fibrin, gelatin, gelatin-methacryloyl (GEL-MA), silk, chitosan, cellulose, polyoligo(ethylene glycol) methyl ether methacrylate (POEGMA), self assembling peptide hydrogels, or a combination thereof.

The term “functionalized alginate” as used herein refers to alginate that is chemically modified to include one or more properties that are advantageous in the manufacture of a fiber of the present disclosure. Examples of functionalized alginates include but are not limited to methacrylated alginate, alginate furan, alginate thiol, alginate maleimide, RGD-alginate, YIGSR-alginate, and covalent click alginates (e.g., alginate blended with DMAPS-Ald and/or DMAPS-Hzd).

The term “immunoprotective” as used herein refers broadly to a design aspect of a fiber of the present disclosure that serves to reduce, prevent or eliminate the host immune response including, e.g., immune cell invasion of the fiber upon implantation of the fiber into a body (e.g., mammalian body). In some embodiments the external shell layer of the present disclosure may be free from any cellular material. In some embodiments the external shell layer may be comprised of a hydrogel material, for example a hydrogel material comprising one or more of alginate, chitosan, GEL-MA, PEG, PEDGA, multi-arm PEG acrylate (PEGTA and PEFOA) (and other PEG-based materials), POEGMA, methacrylated hyaluronic acid, thiolated hyaluronic acid, DMAPS-Ald, DMAPS-Hzd, poly-L-lysine (PLL), triazole (Qingsheng et al., Biomaterials. 2020; 230:119640), and the like. In some examples, an immunoprotective hydrogel material may comprise, for example, a functionalized alginate.

The term “pro-vasculogenic” as used herein refers broadly to a design aspect of a fiber of the present disclosure that serves to encourage blood vessel growth into and/or around a fiber of the present disclosure. Pro-vasculogenic materials may comprise biodegradable materials including but not limited to fibrin, collagen, gelatin, GEL-MA, decellularized extracellular matrix (dECM), polymethacrylic acid, RGD-alginate, YIGSR-alginate, hyaluronic acid (HA), and the like.

The term “agent” as used herein refers to any protein, nucleic acid molecule (including chemically modified nucleic acid molecules), antibody, small molecule, organic compound, inorganic compound, or other molecule of interest. An agent can include a biologically relevant agent, a therapeutic agent, a diagnostic agent or a pharmaceutical agent. A therapeutic or pharmaceutical agent is one that alone or together with an additional compound induces a desired response (such as inducing a therapeutic or prophylactic effect when administered in a manner consistent with the present disclosure to a subject. A biologically relevant agent is one that supports another biological process, for example an agent that supports cell viability.

### Input Materials:

Aspects of the invention include input materials that can be used for printing fiber structures. In some embodiments, an input material comprises a hydrogel. Non-limiting examples of hydrogels include alginate, agarose, collagen, fibrinogen, gelatin, chitosan, hyaluronic acid based gels, or any combination thereof. A variety of synthetic hydrogels are known and can be used in embodiments of the systems and methods provided herein. For example, in some embodiments, one or more hydrogels form at least part of the structural basis for three dimensional structures that are printed. In some embodiments, a hydrogel has the capacity to support growth and/or proliferation of one or more cell types, which may be dispersed within the hydrogel or added to the hydrogel after it has been printed in a three dimensional configuration. In some embodiments, a hydrogel is cross-linkable by a chemical cross-linking agent. For example, a hydrogel comprising alginate may be cross-linkable in the presence of a divalent cation, a hydrogel containing chitosan may be cross-linked using a polyvalent anion such as sodium tripolyphosphate (STP), a hydrogel comprising fibrinogen may be cross-linkable in the presence of an enzyme such as thrombin, and a hydrogel comprising collagen, gelatin, agarose or chitosan may be cross-linkable in the presence of heat or a basic solution. In some embodiments hydrogel fibers may be generated through a precipitation reaction achieved via solvent extraction from the input material upon exposure to a cross-linker material that is miscible with the input material. Non-limiting examples of input materials that form fibers via a precipitation reaction include collagen and polylactic acid (PLA). Non-limiting examples of cross-linking materials that enable precipitation-mediated hydrogel fiber formation include polyethylene glycol (PEG) and alginate. Cross-linking of the hydrogel will increase the hardness of the hydrogel, in some embodiments allowing formation of a solidified hydrogel.

In some embodiments, a hydrogel comprises alginate. Alginate forms solidified colloidal gels (high water content gels, or hydrogels) when contacted with divalent cations. Any suitable divalent cation can be used to form a solidified hydrogel with an input material that comprises alginate. In the alginate ion affinity series Cd²⁺>Ba²⁺>Cu²⁺>Ca²⁺>Ni²⁺>Co²⁺>Mn²⁺, Ca²⁺ is the best characterized and most used to form alginate gels (Ouwerx, C. et al., Polymer Gels and Networks, 1998, 6(5):393-408). Studies indicate that Ca-alginate gels form via a cooperative binding of Ca²⁺ ions by poly G blocks on adjacent polymer chains, the so-called “egg-box” model (ISP Alginates, Section 3: Algin-Manufacture and Structure, in Alginates: Products for Scientific Water Control, 2000, International Specialty Products: San Diego, pp. 4-7). G-rich alginates tend to form thermally stable, strong yet brittle Ca-gels, while M-rich alginates tend to form less thermally stable, weaker but more elastic gels. In some embodiments, a hydrogel comprises a depolymerized alginate.

In some embodiments, a hydrogel is cross-linkable using a free-radical polymerization reaction to generate covalent bonds between molecules. Free radicals can be generated by exposing a photoinitiator to light (often ultraviolet), or by exposing the hydrogel precursor to a chemical source of free radicals such as ammonium peroxodisulfate (APS) or potassium peroxodisulfate (KPS) in combination with N,N,N,N-Tetramethylethylenediamine (TEMED) as the initiator and catalyst respectively. Non-limiting examples of photo cross-linkable hydrogels include: methacrylated hydrogels, such as gelatin methacrylate (GEL-MA) or polyethylene (glycol) acrylate-based (PEG-Acylate) hydrogels, which are used in cell biology due to their ability to cross-link in presence of free radicals after exposure to UV light and due to their inertness to cells. Polyethylene glycol diacrylate (PEG-DA) is commonly used as scaffold in tissue engineering, since polymerization occurs rapidly at room temperature and requires low energy input, has high water content, is elastic, and can be customized to include a variety of biological molecules.

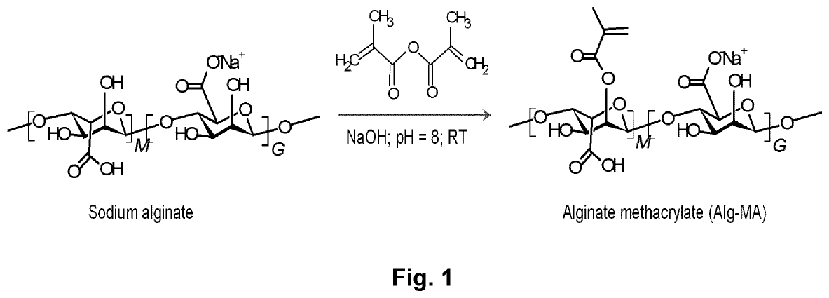

In some embodiments, a hydrogel comprises a chemically modified alginate. In examples, the chemically modified alginate comprises alginate functionalized with methacrylate groups, referred to herein as “Alg-MA” (FIG. 1). In some embodiments, the Alg-MA can be used in an immunoprotective shell layer via blending with zwitterionic alginate, referred to herein as “Alg-zw.” Because of a dual cross-linking capability of Alg-MA, in embodiments the Alg-MA may be first printed with Alg-zw via physical cross-linking. Upon printing the fibers can then be further irradiated to induce covalent cross-linking across fibers thus resulting in fiber-to-fiber (F-F) adhesion. In some embodiments, the chemically modified alginate may comprise thiolated alginate. Other examples of functionalized alginates include alginate furan, alginate maleimide, alginate tetrazine, alginate norbornene, alginate hydrazide, RGD-alginate, and YIGSR-alginate.

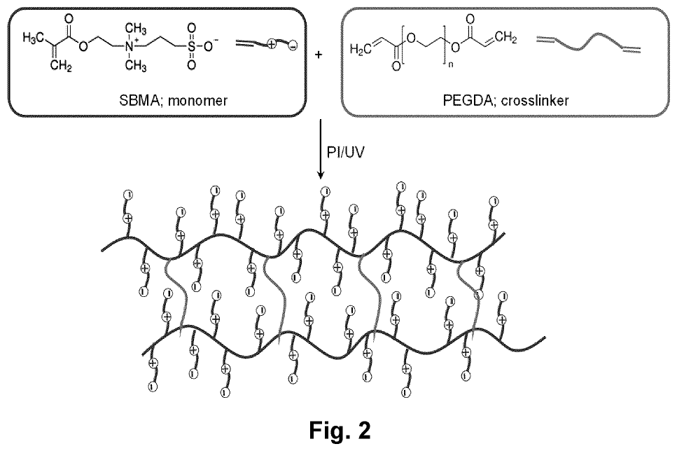

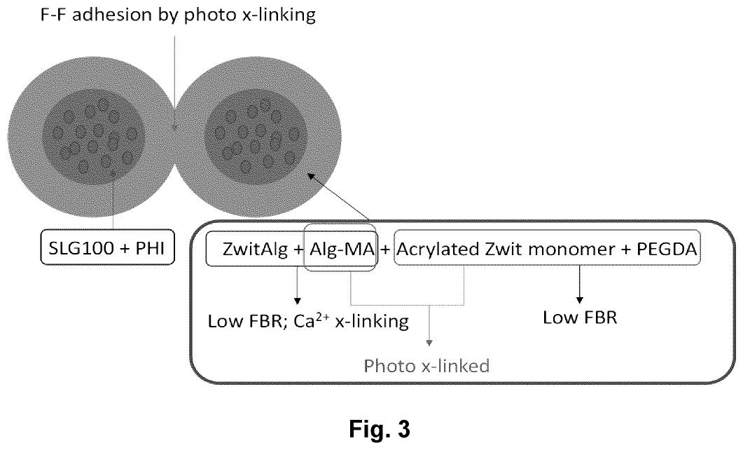

In some embodiments, one or more synthetic components may be added into hydrogel materials. Synthetic components may be useful in increasing F-F adhesion and/or in vivo stability. In examples, a shell material may comprise an acrylated zwitterionic monomer (e.g., sulfobetaine methacrylate (SBMA) and a cross-linker (e.g., poly(ethylene glycol) diacrylate (PEGDA). In such an example, photomediated cross-linking of the zwitterionic monomer with PEGDA may render the resultant cross-linked polymer matrix (FIG. 2) superhydrophilic, and hence, less prone to foreign body response (FBR). An example printing strategy and cross-linking mechanism is illustratively depicted at FIG. 3.

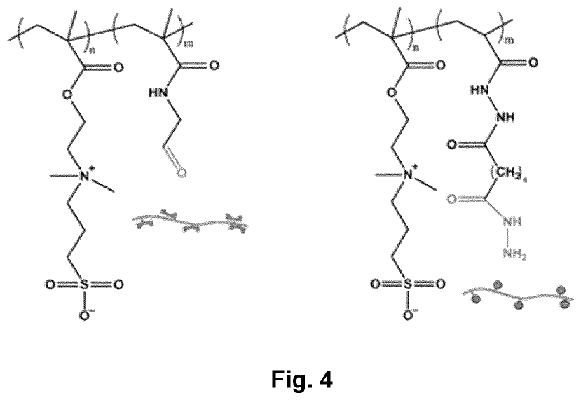

In some embodiments, hydrogel materials may be cross-linked via click chemistry. For example, copolymers comprising a zwitterionic monomer and aldehyde motifs (e.g., [2-(Methacryloyloxy)ethyl]dimethyl-(3-sulfopropyl)ammonium hydroxide (DMAPS)-aldehyde, referred to herein as “DMAPS-Ald”), and zwitterionic monomer and hydrazide motifs (e.g., DMAPS-hydrazide, referred to herein as “DMAPS-Hzd”), may be used. Chemical structures of DMAPS-Ald (also referred to as Zwitt A) and DMAPS-Hzd (also referred to as Zwitt H) are depicted at FIG. 4. Aldehyde reacts readily with hydrazide, forming covalently crosslinked hydrogels. Because of the presence of zwitterionic monomer in the polymer backbone, these polymers may exhibit low protein binding properties. In embodiments, one of these polymers may be blended with alginate in a shell. Following printing, the structure may be submersed in a solution containing the counter component that will in turn result in a covalently cross-linked bridge between fibers leading to F-F adhesion.

In some embodiments, an input material can comprise a non-biodegradable polymer. In examples the input material may be a synthetic polymer, for example polyvinyl acetate (PVA). In some embodiments, an input material may comprise hyaluronic acid (HA).

In some embodiments, an input material may comprise self-assembling peptides. Discussed herein, self-assembling peptides refers to materials comprising monomers of short amino acid sequences or repeated amino acid sequences that assemble to form nanostructures. Peptide assemblies show distinctive physicochemical and biochemical activities, depending on their morphology, size, and accessibility of the reactive surface area (Lee S et al., Int J Mol Sci. (2019); 23: 5850; Yu Z, et al., Curr. Pharm. Des. (2015); 21: 4342-4354; Kisiday J, et al., Proc. Natd. Acad. Sci. USA. (2002); 99:9996. doi: 10.1073/pnas.142309999; Xing R, et al., Biomacromolecules. (2017); 18: 3514-3523).

Table 1 below is a list of biomaterials encompassed by the present disclosure. It should be understood that other materials may be used in the fibers and methods of construction thereof of the present disclosure. Table 1 includes not just input materials, but additional materials as discussed in further detail below.

TABLE 1
Biomaterials used in fiber generation

| | |
|---|---|
| Biomaterials | Source |

Natural

| | |
|---|---|
| Alginate | Commercial (NovaMatrix, Kimica) |
| Alginate furan | Synthesized in-house |
| Alginate maleimide | Synthesized in-house |
| Alginate tetrazine | Synthesized in-house |
| Alginate norbornene | Synthesized in-house |
| Alginate thiol | Synthesized in-house |
| Alginate methacrylate | Synthesized in-house |
| Zwitterionically modified alginate | Synthesized in-house |
| with carboxybetaine | |
| Zwitterionically modified alginate | Synthesized in-house |
| with sulfobetaine | |
| Alginate hydrazide | Synthesized in-house |
| Agarose | Commercial |
| Chitosan | Commercia (OligoMedic) |
| Quaternized chitosan | Synthesized in-house |
| Gelatin | Commercial (Sigma Aldrich) |
| Gelatin methacrylate | Synthesized in-house |
| Gelatin thiol | Synthesized in-house |
| Hyaluronic acid | Commercial (Advanced BioMatrix) |
| Methacrylated hyaluronic acid | Commercial (Advanced BioMatrix) |
| Thiolated hyaluronic acid | Commercial (Advanced BioMatrix) |
| Collagen I | Commercial |
| Fibrinogen | Commercial |
| Laminin | Commercial |
| Decellularized extracellular matrix | Synthesized in house & |
| | commercial |
| Fucoidan | Commercial |

Synthetic (Non-biodegradable)

| | |
|---|---|
| PEGDA | Commercial (Jenkem Technology) |
| 4-arm PEG acrylate (PEGTA) | Commercial (Jenkem Technology) |
| 8-arm PEG acrylate (PEGOA) | Commercial (Jenkem Technology) |
| 4-arm PEG thiol | Commercial (Jenkem Technology) |
| 4-arm PEG maleimide | Commercial (Jenkem Technology) |
| 4-arm PEG vinyl sulfone | Commercial (Jenkem Technology) |
| 4-arm PEG norbornene | Commercial (Jenkem Technology) |
| PVA acrylamide | BioCure |
| [2-(Methacryloyloxy)ethyl]dimethyl- | Todd Hoare Lab (McMaster |
| (3-sulfopropyl)ammonium hydroxide | University, Hamilton, Ontario) |
| (DMAPS)-aldehyde (DMAPS-Ald) | |
| [2-(Methacryloyloxy)ethyl]dimethyl- | Todd Hoare Lab (McMaster |
| (3-sulfopropyl)ammonium hydroxide | University, Hamilton, Ontario) |
| (DMAPS)-hydrazide (DMAPS-Hzd) | |
| Polymethacrylic acid | Synthesized in-house & |
| | commercial |

### Additional Components:

Input materials in accordance with embodiments of the invention can comprise any of a wide variety of natural or synthetic polymers that support the viability of living cells, including, e.g., alginate, laminin, fibrin, hyaluronic acid, poly(ethylene) glycol based gels, gelatin, chitosan, agarose, or combinations thereof. In some embodiments, the subject bioink compositions are physiologically compatible, i.e., conducive to cell growth, differentiation and communication. In certain embodiments, an input material comprises one or more physiological matrix materials, or a combination thereof. By “physiological matrix material” is meant a biological material found in a native mammalian tissue. Non-limiting examples of such physiological matrix materials include: fibronectin, thrombospondin, glycosaminoglycans (GAG) (e.g., hyaluronic acid, chondroitin-6-sulfate, dermatan sulfate, chondroitin-4-sulfate, or keratin sulfate), deoxyribonucleic acid (DNA), adhesion glycoproteins, and collagen (e.g., collagen I, collagen II, collagen III, collagen IV, collagen V, collagen VI, or collagen XVIII).

### Therapeutic Cell Populations

Input materials in accordance with embodiments of the invention can incorporate any mammalian cell type, including but not limited to stem cells (e.g., embryonic stem cells, adult stem cells, induced pluripotent stem cells), germ cells, endoderm cells (e.g., lung, liver, pancreas, gastrointestinal tract, or urogenital tract cells), mesoderm cells (e.g., kidney, bone, muscle, endothelial, or heart cells), ectoderm cells (skin, nervous system, pituitary, or eye cells), stem cell-derived cells, or any combination thereof.

For example, an input material can comprise cells from endocrine and exocrine glands including pancreas (alpha, beta, delta, epsilon, gamma), liver (hepatocyte, Kuppfer, Stelate, sinusoidal cells), thyroid (Follicular cells), pineal gland (pinealocytes), pituitary gland (somatotropes, Lactotropes, gonadotropes, corticotropes, and thyrotropes), thymus (thymocytes, thymic epithelial cells, thymic stromal cells), adrenal gland (cortical cells, chromaffin cells), ovary (granulosa cells), testis (Leydig cells), gastrointestinal tract (enteroendocrine cells—intestinal, gastric, pancreatic), fibroblasts, chondrocytes, meniscus fibrochondrocytes, bone marrow stromal (stem) cells, embryonic stem cells, mesenchymal stem cells, induced pluripotent stem cells, differentiated stem cells, tissue-derived cells, smooth muscle cells, skeletal muscle cells, cardiac muscle cells, epithelial cells, endothelial cells, myoblasts, chondroblasts, osteoblasts, osteoclasts, and any combinations thereof.

Cells can be obtained from donors (allogenic) or from recipients (autologous). Specifically, in embodiments, cells can be obtained from a suitable donor, such as a human or animal, or from the subject into which the cells are to be implanted. Mammalian species include, but are not limited to, humans, monkeys, dogs, cows, horses, pigs, sheep, goats, cats, mice, rabbits, and rats. In one embodiment, the cells are human cells. In other embodiments, the cells can be derived from animals such as dogs, cats, horses, monkeys, or any other mammal.

In some embodiments, an input material can comprise: human cadaveric pancreatic islets, stem cell-derived β-cells, xenogenic pancreatic islets, and isolated human or xenogenic β-cells. In some embodiments, the therapeutic cell or cell population is a pancreatic progenitor cell or population, or a PDX1-positive pancreatic progenitor cell or population, or an endocrine precursor cell or population, or a poly or singly-hormonal endocrine cell and/or any combination thereof, including purified or enriched cells or populations of cells thereof.

In some embodiments, an input material can comprise: human cadaveric liver hepatocytes and/or cholangiocyte cells, stem cell-derived hepatocytes and chondrocytes, or xenogenic liver cells. In some embodiments, the therapeutic cell or cell population is a hepatocyte progenitor cell or population, with or without a mesenchymal cell, including fibroblasts, MSCs.

In some embodiments, the at least one biological material comprises a cell population expressing/secreting one or more endogenous biologically active agent(s), e.g., insulin, glucagon, ghrelin, pancreatic polypeptide, an angiogenic factor, a growth factor, a hormone, an antibody, an enzyme, a protein, an exosome, and the like. Discussed herein, endogenous biologically active agents comprise those agents that the cell naturally produces in a biological context (e.g., insulin release in response to elevated glucose concentrations). An endogenous biologically active agent can constitute a therapeutic agent in the context of the present disclosure.

In some embodiments, an input material can comprise genetically engineered cells that secrete specific factors. It is within the scope of this disclosure that a cell population as discussed above can comprise, in embodiments, engineered cells (e.g., genetically engineered cells) that secrete specific factors. Cells can also be from established cell culture lines, or can be cells that have undergone genetic engineering and/or manipulation to achieve a desired genotype or phenotype. In some embodiments, pieces of tissue can also be used, which may provide a number of different cell types within the same structure.

Genetic engineering techniques applicable to the present disclosure can include but are not limited to recombinant DNA (rDNA) technology (Stryjewska et al., Pharmacologial Reports. 2013; 65: 1075), cell-engineering based on use of targeted nucleases (e.g., meganuclease, zinc finger nucleases (ZFN), transcription activator-like effector nucleases (TALEN), clustered regularly interspaced short palindromic repeat-associated nuclease Cas₉ (CRISPR-Cas₉), etc. (Lim et al., Nature Communications. 2020; 11: 4043; Stoddard B L, Structure. 2011; 19(1): 7-15; Gaj et al., Trends Biotechnol. 2013; 31(7): 397-405; Hsu et al., Cell. 2014; 157(6): 1262; Miller et al., Nat Biotechnol. 2010; 29(2): 143-148), cell-engineering based on use of site-specific recombination using recombinase systems (e.g., Cre-Lox) (Osborn et al., Mol Ther. 2013; 21(6): 1151-1159; Hockemeyer et al., Nat Biotechnol. 2009; 27(9): 851-857; Uhde-Stone et al., RNA. 2014; 20(6): 948-955; Ho et al., Nucleic Acids Res. 2015; 43(3): e17; Sengupta et al., Journal of Biological Engineering. 2017; 11(45): 1-9), and the like. In some embodiments, some combination of the above-mentioned techniques for cell-engineering may be used.

Encompassed by the present disclosure are engineered cells capable of producing one or more therapeutic agents, including but not limited to proteins, peptides, nucleic acids (e.g., DNA, RNA, mRNA, siRNA, miRNA, nucleic acid analogs), peptide nucleic acids, aptamers, antibodies or fragments or portions thereof, antigens or epitopes, hormones, hormone antagonists, growth factors or recombinant growth factors and fragments and variants thereof, cytokines, enzymes, antibiotics or antimicrobial compounds, anti-inflammation agent, antifungals, antivirals, toxins, prodrugs, small molecules, drugs (e.g., drugs, dyes, amino acids, vitamins, antioxidants) or any combination thereof.

In some examples, engineered cells of the present disclosure include cells engineered to release extracellular vesicles (EVs), for example exosomes and microvesicles. Different cell types may produce EVs with distinct cargo (e.g., proteins, RNA, siRNA, and the like), and it is further within the scope of this disclosure that particular cell types may be genetically engineered to produce EVs with particular cargo. It is further within the scope of this disclosure that EVs comprise targeting technologies for enhancing, for example, exosome-mediated delivery to tissues of interest. Such technology may be combined, for example, with inherent biology of exosomes to drive in vivo behavior (Zeh et al., PLOS One. 2019; 14(8): e0221679; Gurung et al., American Society for Cell and Gene Therapy Poster Presentation, 2020; Zickler and Andaloussi, Nature Biomedical Engineering. 2020; 4: 9-11; Zipkin, Nature Biotechnology. 2019; 37(12): 1395-1400; Wiklander et al., Science Translational Medicine. 2019; 11(492): eeav8521; Görgens et al., J Extracell Vesicles. 2019; 8(1): 1587567; Wiklander et al., Front Immunol. 2018; 13(9): 1326; Corso et al., Scientific Reports. 2017; 7: 11561; Cooper et al., Mov Disord. 2014; 29(12): 1476-85; Andaloussi et al., Nature Reviews Drug Discovery. 2013; 12: 347-357; Alvarez-Erviti et al., Nature Biotechnology. 2011; 29: 341-345; US Patent Application No. 2019/0167810; US Patent Application No. 2020/0109183). In some embodiments, EVs may themselves be incorporated into particular layers of fibers of the present disclosure (Kaiqi et al., Theranostics. 2019; 9(24):7403-7416).

In embodiments, therapeutic cells of the present disclosure may be modified to comprise at least one mechanism for providing a local immunosuppression at a transplant site when transplanted in an allogeneic host, for example in tissue fibers of the present disclosure. In examples, a cell or cells may comprise a set of transgenes, each transgene encoding a gene product that is cytoplasmic, membrane bound, or local acting, and whose function can include but is not limited to mitigate antigen presenting cell activation and function; to mitigate graft attacking leukocyte activity or cytolytic function; to mitigate macrophage cytolytic function and phagocytosis of allograft cells; to induce apoptosis in graft attacking leukocytes; to mitigate local inflammatory proteins; and to protect against leukocyte-mediated apoptosis (WO2018/227286; Harding et al., BioRxiv. 2019; DOI: 10.1101/716571; Lanza et al., Nature Reviews Immunology. 2019; 19: 723-7331; Harding et al., Cell Stem Cell. 2020; 27(2): 198-199).

In embodiments, therapeutic cells of the present disclosure may be modified in a manner to exert control over cell proliferation. As an example, a cell may be genetically modified at a cell division locus (CDL) to comprise a negative selectable marker and/or an inducible activator-based gene expression system, thereby enabling control over the permitting, ablation and/or inhibition of proliferation of the genetically modified cells by addition or removal of an appropriate inducer (WO2016/141480; Liang et al., Nature. 2018; 563(7733): 701-704).

Appropriate growth conditions for mammalian cells are well known in the art (Freshney, R. I. (2000) Culture of Animal Cells, a Manual of Basic Technique. Hoboken N.J., John Wiley & Sons; Lanza et al. Principles of Tissue Engineering, Academic Press; 2nd edition May 15, 2000; and Lanza & Atala, Methods of Tissue Engineering Academic Press; 1st edition October 2001). Cell culture media generally include essential nutrients and, optionally, additional elements such as growth factors, salts, minerals, vitamins, etc., that may be selected according to the cell type(s) being cultured. Particular ingredients may be selected to enhance cell growth, differentiation, secretion of specific proteins, etc. In general, standard growth media include Dulbecco's Modified Eagle Medium, low glucose (DMEM), with 110 mg/L pyruvate and glutamine, supplemented with 10-20% fetal bovine serum (FBS) or calf serum and 100 U/ml penicillin are appropriate as are various other standard media well known to those in the art. Growth conditions will vary depending on the type of mammalian cells in use and the tissue desired.

In some embodiments, cell-type specific reagents can be advantageously employed in the subject input materials for use with a corresponding cell type. For example, an extracellular matrix (“ECM”) can be extracted directly from a tissue of interest and then solubilized and incorporated it into an input material to generate tissue-specific input materials for printed tissues. Such ECMs can be readily obtained from patient samples and/or are available commercially from suppliers such as zPredicta (rBone™, available at zpredicta.com/home/products).

### Active Agents:

In some aspects, embodiments of the invention can comprise at least one active agent added to fibers of the present disclosure during printing, e.g. biologically relevant agents to help facilitate cell growth and/or differentiation. Non-limiting examples of such active agents include TGF-β1, TGF-β2, TGF-β3, BMP-2, BMP-4, BMP-6, BMP-12, BMP-13, basic fibroblast growth factor, fibroblast growth factor-1, fibroblast growth factor-2, platelet-derived growth factor-AA, platelet-derived growth factor-BB, platelet rich plasma, IGF-I, IGF-II, GDF-5, GDF-6, GDF-8, GDF-10, vascular endothelial cell-derived growth factor, pleiotrophin, endothelin, nicotinamide, glucagon like peptide-I, glucagon like peptide-II, parathyroid hormone, tenascin-C, tropoelastin, thrombin-derived peptides, laminin, biological peptides containing cell-binding domains and biological peptides containing heparin-binding domains, therapeutic agents, and any combinations thereof.

Additional active agents can include, but are not limited to, proteins, peptides, nucleic acid analogues, nucleotides, oligonucleotides, nucleic acids (DNA, RNA, siRNA, mRNA), peptide nucleic acids, aptamers, antibodies or fragments or portions thereof, antigens or epitopes, hormones, hormone antagonists, growth factors or recombinant growth factors and fragments and variants thereof, cytokines, enzymes, antibiotics or antimicrobial compounds, anti-inflammation agent, antifungals, antivirals, toxins, prodrugs, small molecules, drugs (e.g., drugs, dyes, amino acids, vitamins, antioxidants) or any combination thereof.

Non-limiting examples of anti-inflammatory and anti-fibrotic factors that are suitable for inclusion as an input material include: steroids (dexamethasone), pirfenidone, prostaglandin agonists (butaprost), rapamycin, GW2580, and the like.

Non-limiting examples of antibiotics that are suitable for inclusion in an input material include: aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, fusidic acid, or any combination thereof.

Non-limiting examples of antibodies include: abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, zanolimumab, or any combination thereof.

Non-limiting examples of enzymes suitable for use in an input material as described herein include: peroxidase, lipase, amylose, organophosphate dehydrogenase, ligases, restriction endonucleases, ribonucleases, DNA polymerases, glucose oxidase, and laccase.

Additional non-limiting examples of active agents that are suitable for use with the subject input materials include: cell growth media, such as Dulbecco's Modified Eagle Medium, fetal bovine serum, non-essential amino acids and antibiotics; growth and morphogenic factors such as fibroblast growth factor, transforming growth factors, vascular endothelial growth factor, epidermal growth factor, platelet derived growth factor, insulin-like growth factors), bone morphogenetic growth factors, bone morphogenetic-like proteins, transforming growth factors, nerve growth factors, and related proteins (growth factors are known in the art, see, e.g., Rosen & Thies, CELLULAR & MOLECULAR BASIS BONE FORMATION & REPAIR (R.G. Landes Co., Austin, Tex., 1995); anti-angiogenic proteins such as endostatin, and other naturally derived or genetically engineered proteins; polysaccharides, glycoproteins, or lipoproteins; anti-infectives such as antibiotics and antiviral agents, chemotherapeutic agents (i.e., anticancer agents), anti-rejection agents, analgesics and analgesic combinations, anti-inflammatory agents, steroids, or any combination thereof.

### Printing Systems:

Preferably, the bioprinted tissue fibers described herein are printed using LOP™ technology as described in PCT/CA2014/050556, PCT/CA2018/050315, and U.S. Ser. No. 62/733,548; the disclosures of which are expressly incorporated herein by reference. As detailed therein, the LOP™ bioprinting system enables multi-material switching, and thus the composition of the vessel wall (cell type and biomaterial composition) can be modified along the length of the channel while continuously printing.

In an exemplary embodiment, the printing system comprises a print head comprising a dispensing channel, wherein one or more material channels and a core channel converge at the proximal end of the dispensing channel. The subject print heads may be configured to dispense buffer solution and/or sheath fluid simultaneous with one or more cross-linkable materials. In some embodiments, a print head is configured to maintain a constant mass flow rate through the dispensing channel. In this manner, the subject print heads are configured to facilitate a smooth and continuous flow of one or more input materials (or a mixture of one or more input materials) and a buffer solution and/or sheath fluid through the dispensing channel. In use of the subject print heads, an input material flowing through the dispensing channel can be cross-linked from the inside, by a fluid flowing through the core channel and/or from the outside, by sheath fluid flowing through a downstream sheath fluid channel, as described more particularly in WO2020/056517, the disclosure of which is expressly incorporated herein by reference.

In a preferred embodiment, a print head comprises a dispensing channel with a proximal end and a distal end; a dispensing orifice located at the distal end of the dispensing channel; one or more shell channels that converge sequentially with the dispensing channel at the distal end of the dispensing channel, wherein each shell channel has a convergence angle of between about 20 and 90 degrees, a core channel that converges with the dispensing channel at the proximal end of the dispensing channel, wherein the core channel has a convergence angle of 0 degrees; and, optionally a sheath flow channel that diverges into two sheath flow sub-channels, wherein the sheath flow sub-channels converge with the dispensing channel at a sheath fluid intersection and have a convergence angle of between about 30 and 60 degrees, more preferably between about 40 and 50 degrees, most preferably about 45 degrees.

Additional aspects include printing systems and associated components that are configured to work in conjunction with the subject print heads to prepare the subject fibers. In some embodiments, a printing system comprises a single print head, as described herein. In some embodiments, a printing system comprises a plurality of print heads, such as 2, 3, 4, 5, 6, 7, 8, 9 or 10 individual print heads, as described herein. In some embodiments, a print head is fluidically isolated from a printing system, such that all fluids involved with the printing process remain isolated within the print head, and only make contact with a receiving surface of the printing system (described below) during the printing process. In some embodiments, a print head is configured to be operably coupled to a printing system without bringing the fluids involved with the printing process into contact with the components of the printing system. In some embodiments, one or more print heads can be removed and/or added to a printing system before, during and/or after a printing process. Accordingly, in some embodiments, the subject print heads are modular components of the subject printing systems.

In some embodiments, a printing system comprises a receiving surface upon which a first layer of material dispensed from a dispensing orifice of a print head is deposited. In some embodiments, a receiving surface comprises a solid material. In some embodiments, a receiving surface comprises a porous material. For example, in some embodiments, the porosity of the porous material is sufficient to allow passage of a fluid there through. In some embodiments, a receiving surface is substantially planar, thereby providing a flat surface upon which a first layer of dispensed material can be deposited. In some embodiments, a receiving surface has a topography that corresponds to a three dimensional structure to be printed, thereby facilitating printing of a three dimensional structure having a non-planar first layer.

In some embodiments, a receiving surface comprises a vacuum component that is configured to apply suction from one or more vacuum sources to the receiving surface. In some embodiments, a receiving surface comprises one or more vacuum channels that are configured to apply suction to the receiving surface. In some embodiments, a receiving surface comprising a vacuum component is configured to aspirate an excess fluid from the receiving surface before, during and/or after a printing process is carried out.

In some embodiments, a printing system achieves a particular geometry by moving a print head relative to a printer stage or receiving surface adapted to receive printed materials. In other embodiments, a printing system achieves a particular geometry by moving a printer stage or receiving surface relative to a print head. In certain embodiments, at least a portion of a printing system is maintained in a sterile environment (e.g., within a biosafety cabinet (BSC)). In some embodiments, a printing system is configured to fit entirely within a sterile environment.

In some embodiments, a printing system comprises a 3D motorized stage comprising three arms for positioning a print head and a dispensing orifice in three dimensional space above a print bed, which comprises a surface for receiving a printed material. In one embodiment, the 3D motorized stage (i.e., the positioning unit) can be controlled to position a vertical arm, which extends along the z-axis of the 3D motorized stage such that the print head orifice is directed downward. A first horizontal arm, which extends along the x-axis of the motorized stage is secured to an immobile base platform. A second horizontal arm, which extends along the y-axis of the motorized stage is moveably coupled to an upper surface of the first horizontal arm such that the longitudinal directions of the first and second horizontal arms are perpendicular to one another. It will be understood that the terms “vertical” and “horizontal” as used above with respect to the arms are meant to describe the manner in which the print head is moved and do not necessarily limit the physical orientation of the arms themselves.

In some embodiments, a receiving surface is positioned on top of a platform, the platform being coupled to an upper surface of the second horizontal arm. In some embodiments, the 3D motorized stage arms are driven by three corresponding motors, respectively, and controlled by a programmable control processor, such as a computer. In a preferred embodiment, a print head and a receiving surface are collectively moveable along all three primary axes of a Cartesian coordinate system by the 3D motorized stage, and movement of the stage is defined using computer software. It will be understood that the invention is not limited to only the described positioning system, and that other positioning systems are known in the art. As material is dispensed from a dispensing orifice on a print head, the positioning unit is moved in a pattern controlled by software, thereby creating a first layer of the dispensed material on the receiving surface. Additional layers of dispensed material are then stacked on top of one another such that the final 3D geometry of the dispensed layers of material is generally a replica of a 3D geometry design provided by the software. The 3D design may be created using typical 3D CAD (computer aided design) software or generated from digital images, as known in the art. Further, if the software generated geometry contains information on specific materials to be used, it is possible, according to one embodiment of the invention, to assign a specific input material type to different geometrical locations. For example, in some embodiments, a printed 3D structure can comprise two or more different input materials, wherein each input material has different properties (e.g., each input material comprises a different cell type, a different cell concentration, a different ECM composition, etc.).

Aspects of the subject printing systems include software programs that are configured to facilitate deposition of the subject input materials in a specific pattern and at specific positions in order to form a specific fiber, planar or 3D structure. In order to fabricate such structures, the subject printing systems deposit the subject input materials at precise locations (in two or three dimensions) on a receiving surface. In some embodiments, the locations at which a printing system deposits a material are defined by a user input, and are translated into computer code. In some embodiments, a computer code includes a sequence of instructions, executable in the central processing unit (CPU) of a digital processing device, written to perform a specified task. In some embodiments, printing parameters including, but not limited to, printed fiber dimensions, pump speed, movement speed of the print head positioning system, and cross-linking agent intensity or concentration are defined by user inputs and are translated into computer code. In some embodiments, printing parameters are not directly defined by user input, but are derived from other parameters and conditions by the computer code.

In some embodiments, the locations at which a printing system deposits an input material are defined by a user input and are translated into computer code. In some embodiments, the devices, systems, and methods disclosed herein further comprise non-transitory computer readable storage media or storage media encoded with computer readable program code. In some embodiments, a computer readable storage medium is a tangible component of a digital processing device such as a bioprinter (or a component thereof) or a computer connected to a bioprinter (or a component thereof). In some embodiments, a computer readable storage medium is optionally removable from a digital processing device. In some embodiments, a computer readable storage medium includes, by way of non-limiting example, a CD-ROM, DVD, flash memory device, solid state memory, magnetic disk drive, magnetic tape drive, optical disk drive, cloud computing system and/or service, and the like. In some cases, the program and instructions are permanently, substantially permanently, semi-permanently, or non-transitorily encoded on a storage medium.

In some embodiments, the devices, systems, and methods described herein comprise software, server, and database modules. In some embodiments, a “computer module” is a software component (including a section of code) that interacts with a larger computer system. In some embodiments, a software module (or program module) comes in the form of one or more files and typically handles a specific task within a larger software system.

In some embodiments, a module is included in one or more software systems. In some embodiments, a module is integrated with one or more other modules into one or more software systems. A computer module is optionally a stand-alone section of code or, optionally, code that is not separately identifiable. In some embodiments, the modules are in a single application. In other embodiments, the modules are in a plurality of applications. In some embodiments, the modules are hosted on one machine. In some embodiments, the modules are hosted on a plurality of machines. In some embodiments, the modules are hosted on a plurality of machines in one location. In some embodiments, the modules are hosted a plurality of machines in more than one location. Computer modules in accordance with embodiments of the invention allow an end user to use a computer to perform the one or more aspects of the methods described herein.

In some embodiments, a computer module comprises a graphical user interface (GUI). As used herein, “graphic user interface” means a user environment that uses pictorial as well as textual representations of the input and output of applications and the hierarchical or other data structure in which information is stored. In some embodiments, a computer module comprises a display screen. In further embodiments, a computer module presents, via a display screen, a two-dimensional GUI. In some embodiments, a computer module presents, via a display screen, a three-dimensional GUI such as a virtual reality environment. In some embodiments, the display screen is a touchscreen and presents an interactive GUI.

Aspects also include one or more quality control components that are configured to monitor and/or regulate one or more parameters of the subject printing systems in order to ensure that one or more printed fibers have suitable properties. For example, in some embodiments, if a deposition process proceeds too quickly, a printed fiber structure can begin to form a coiled structure within the dispensing channel or outside the dispensing channel after it has been dispensed. In some embodiments, a quality control component comprises a camera that is configured to monitor the deposition process by collecting one or more images of a printed fiber structure, and to determine whether the printed fiber structure has formed a coiled structure. In some embodiments, a quality control component is configured to modulate one or more parameters of a deposition process (e.g., to reduce pressure and/or to reduce deposition speed) so as to diminish or avoid formation of a coiled structure by the printed fiber structure.

Aspects further include one or more fluid reservoirs that are configured to store a fluid and deliver the fluid to the printing system (e.g., the print head) through one or more fluid channels, which provide fluid communication between the printing system and the reservoirs. In some embodiments, a printing system comprises one or more fluid reservoirs that are in fluid communication with a fluid channel. In some embodiments, a fluid reservoir is connected to an input orifice of a fluid channel. In some embodiments, a fluid reservoir is configured to hold a volume of fluid that ranges from about 100 μL up to about 1 L, such as about 1, 5, 10, 15, 20, 25, 30, 35, 40, 45, 50, 55, 60, 65, 70, 75, 80, 85, 90, 95 or 100 mL, or such as about 150, 200, 250, 300, 350, 400, 450, 500, 550, 600, 650, 700, 750, 800, 850, 900 or 950 mL.

In some embodiments, a printing system comprises a pressure control unit, which is fluidly coupled to the one or more reservoirs. The pressure control unit is configured to provide a force to move one or more fluids through the printing system. In some embodiments, a pressure control unit supplies pneumatic pressure to one or more fluids via one or more connecting tubes. The pressure applied forces a fluid out of a reservoir and into the print head via respective fluid channels. In some embodiments, alternative means can be used to move a fluid through a channel. For example, a series of electronically controlled syringe pumps could be used to provide force for moving a fluid through a print head.

In some embodiments, a printing system comprises a light module (as described above) for optionally exposing a photo cross-linkable input material to light in order to cross-link the material. Light modules in accordance with embodiments of the invention can be integrated into a print head, or can be a component of a printing system.

### Additional Fluids:

Aspects of the invention include one or more buffer solutions. Buffer solutions in accordance with embodiments of the invention are miscible with an input material (e.g., a hydrogel) and do not cross-link the input material. In some embodiments, a buffer solution comprises an aqueous solvent. Non-limiting examples of buffer solutions include polyvinyl alcohol, water, glycerol, propylene glycol, sucrose, gelatin, or any combination thereof.

Buffer solutions in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa·s. In some embodiments, the viscosity of a buffer solution can be modulated so that it matches the viscosity of one or more input materials.

Aspects of the invention include one or more sheath fluids. Sheath fluids in accordance with embodiments of the invention are fluids that can be used, at least in part, to envelope or “sheath” an input material being dispensed from a dispensing channel. In some embodiments, a sheath fluid comprises an aqueous solvent. Non-limiting examples of sheath fluids include polyvinyl alcohol, water, glycerol, propylene glycol, sucrose, gelatin, or any combination thereof. Sheath fluids in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa·s. In some embodiments, the viscosity of a sheath fluid can be modulated so that it matches the viscosity of one or more input materials.

In some embodiments, a sheath fluid comprises a chemical cross-linking agent. In some embodiments, a chemical cross-linking agent comprises a divalent cation. Non-limiting examples of divalent cations include Cd²⁺, Ba²⁺, Cu²⁺, Ca²⁺, Ni²⁺, Co²⁺, or Mn²⁺. In a preferred embodiment, Ca²⁺ is used as the divalent cation. In some embodiments, the concentration of a divalent cation in the sheath fluid ranges from about 80 mM to about 140 mM, such as about 90, 100, 110, 120 or 130 mM.

### Methods of Use:

Aspects of the invention include methods of printing a linear fiber structure, a planar structure comprising one or more fiber structures, or a three-dimensional (3D) structure comprising two or more layers of planar structures. In some embodiments, a method first comprises providing a design for a planar or 3D structure to be printed. The design can be created using commercially available CAD software. In some embodiments, the design comprises information regarding specific materials (e.g., for heterogeneous structures comprising multiple materials) to be assigned to specific locations in the structure(s) to be printed.

Aspects of the methods comprise providing one or more input materials to be dispensed by the print head. In some embodiments, one or more cell types are compatible with, and optionally dispensed within, an input material. In some embodiments, a sheath fluid serves as a lubricating agent for lubricating movement of an input material within the print head. In some embodiments, a sheath fluid comprises a cross-linking agent for solidifying at least a portion of the hydrogel before or while it is dispensed from the print head. In some embodiments, a cross-linking agent may be included in an input material, for example an input material corresponding to a core of a fiber of the present disclosure.

Aspects of the methods comprise communicating the design to the 3D printer. In some embodiments, communication can be achieved, for example, by a programmable control processor. In some embodiments, the methods comprise controlling relative positioning of the print head and the receiving surface in three dimensional space, and simultaneously dispensing from the print head the input material, and, in some embodiments, a sheath fluid, alone or in combination. In some embodiments, the materials dispensed from the print ahead are dispensed coaxially, such that the sheath fluid envelopes the input material. Such coaxial arrangement allows a cross-linking agent in the sheath fluid to solidify the input material, thereby resulting in a solidified fiber structure, which is dispensed from the printer head.

In some embodiments, a method comprises depositing a first layer of the dispensed fiber structure on a receiving surface, the first layer comprising an arrangement of the fiber structure specified by the design, and iteratively repeating the depositing step, depositing subsequent fiber structures onto the first and subsequent layers, thereby depositing layer upon layer of dispensed fiber structures in a geometric arrangement specified by the design to produce a 3D structure.

In some embodiments, a plurality of input materials, for example multiple hydrogels, at least some of which comprise one or more cell types, are deposited in a controlled sequence, thereby allowing a controlled arrangement of input materials and cell types to be deposited in a geometric arrangement specified by the design.

In some embodiments, a method comprises removing excess fluid from the receiving surface and optionally from the surface of the dispensed fiber structure. For example, the step of removing the excess fluid can be done continuously throughout the printing process, thereby removing excess fluid that may otherwise interfere with layering the dispensed fiber structures in the geometric arrangement provided by the design. Alternatively, the step of removing excess fluid can be done intermittently throughout the printing process in sequence with or simultaneously with one or more depositing steps. In some embodiments, removal of excess fluid is achieved by drawing the fluid off of the receiving surface and optionally off of a surface of a dispensed fiber structure. In some embodiments, removal of excess fluid is achieved by drawing excess fluid through the receiving surface, the receiving surface comprising pores sized to allow passage of the fluid. In some embodiments, removal of excess fluid is achieved by providing a fluid that evaporates after being dispensed from the dispensing orifice.

Aspects of the invention include methods of making a 3D structure comprising one or more input materials. The 3D structures find use in repairing and/or replacing at least a portion of a damaged or diseased tissue in a subject.

As described above, any suitable divalent cation can be used in conjunction with the subject methods to solidify a chemically cross-linkable input material, including, but not limited to, Cd²⁺, Ba²⁺, Cu²⁺, Ca²⁺, Ni²⁺, Co²⁺, or Mn²⁺. In a preferred embodiment, Ca²⁺ is used as the divalent cation. In one preferred embodiment, a chemically cross-linkable input material is contacted with a solution comprising Ca²⁺ to form a solidified fiber structure. In some embodiments, the concentration of Ca²⁺ in the sheath solution ranges from about 80 mM to about 140 mM, such as about 90, 100, 110, 120 or 130 mM.

In certain embodiments, an input material is solidified in less than about 5 seconds, such as less than about 4 seconds, less than about 3 seconds, less than about 2 seconds, or less than about 1 second.

Aspects of the invention include methods of depositing one or more input materials in a patterned manner, using software tools, to form layers of solidified structures that are formed into a multi-layered 3D tissue structure. In some embodiments, a multi-layered 3D tissue structure comprises a plurality of mammalian cells. Advantageously, by modulating the components (e.g., the mammalian cell type, cell density, matrix components, active agents) of the subject input materials, a multi-layered 3D tissue structure can be created using the subject methods, wherein the multi-layered 3D tissue structure has a precisely controlled composition at any particular location in three dimensional space. As such, the subject methods facilitate production of complex three dimensional tissue structures.

### Methods of Making:

### Outside-In Cross-Linking

Extrusion-based bioprinting methods generally involve the generation of fibers formed from hydrogels and other biomaterials that are cross-linked upon deposition using a variety of mechanisms that depend on the type of biomaterial being extruded. For example, extruded fibers are printed into a bath of cross-linker material that causes the fiber to solidify. The fiber is then collected from the bath, for example on a mandrel, and then may be used in a variety of applications which may include tissue engineering applications.

By contrast, microfluidic bioprinting, such as that disclosed herein, reduces the need to print into a liquid bath since the cross-linker material can be coextruded in a coaxial fashion surrounding the material of interest, causing the gelling process to begin immediately upon deposition. Microfluidic printing of cross-linkable cytocompatible hydrogels such as alginate with living cells, as discussed herein, is enabled by cationic cross-linking using cytocompatible concentrations of CaCl₂ or other cationic solution. It is within the scope of this disclosure that cross-linking can be performed using an “outside-in” approach where in its simplest form, channels in the print head direct one stream of material, such as alginate, to be surrounded in a sheath of cross-linker material. As discussed herein, more complex microfluidic print head architectures can be developed, leveraging the properties of laminar flow to generate multi-layer core-shell fibers with multiple layers of shell material surrounding a central core. In outside-in cross-linking, the Ca²⁺ ions diffuse from the sheath material into the core, cross-linking the alginate, and thus the outermost layer of the fiber solidifies almost instantaneously, while the core solidifies over some period of time. In some instances, patterning of an outside-in cross-linked fiber to build a tissue construct can be challenging, and particularly when fibers deposited onto a previous layer do not effectively fuse with the previous layer causing them to slip during deposition. This can make patterning a structure accurately and consistently, challenging. That said, in embodiments, devices of the present disclosure may be made, at least in part, via the process of outside-in cross-linking.

### Inside-Out Cross-Linking

In embodiments, devices of the present disclosure can be made via an inside-out cross-linking approach. In an inside-out crosslinking approach, an ionic cross-linker (e.g., CaCl₂) may be included along with an input material that forms a solid core, at similar concentrations that would otherwise be used in an outside-in approach. For example, one or more shells surrounding the core may be alginate-based, and the one or more shells may be cross-linked from the inside out as the ions diffuse outward from the non-alginate core. In such an example, the outermost surface of the outer shell is cross-linked last. This may result in the outer surface of the fiber remaining “sticky” when it is extruded from the print head. When these sticky fibers are deposited onto a previous layer that is also in the process of solidifying, the outer surfaces of adjacent fibers may effectively fuse together. In this way, control in the fabrication of 3D macrostructures formed from fibers of the present disclosure may be improved, as compared to similar methodology where the individual fibers are cross-linked using the outside-in approach.

In one example, a core of a fiber device may comprise a synthetic material (e.g., PVA), and the core material may be surrounded by one or more shell materials, for example an inner shell material comprising an annulus layer, and an external shell comprising an immunoprotective layer. The one or more shells may, for example, be alginate-based. In this example, the PVA would be unaffected by the ionic cross-linker (e.g., CaCl₂), and would be cross-linked by other mechanisms, e.g. photo crosslinking. As the ions diffuse outward from the core, the annulus layer material would be cross-linked first, followed by cross-linking of the external shell. Such an example is meant to be representative and non-limiting.

In examples, a fiber device may comprise a core comprised of a biological material surrounded by one or more shells, at least one of which may comprise an immunoprotective and/or vasculogenic layer. In such an example, inside-out cross-linking may similarly be conducted. However, in contrast to the example where the core comprises a synthetic material, in this example the core would comprise a biocompatible material, such as alginate.

In examples of single core, multi-shell fiber devices, inside-out cross-linking may be conducted via the use of a single ionic cross-linker, for example CaCl₂ in a case where the shell layers surrounding the core are each comprised of alginate. In other examples, more than one cross-linker may be used in an inside-out cross-linking approach. As a representative example, a core may be synthetic-based, an internal shell (e.g., annulus layer) may be alginate-based, and an external shell may be chitosan-based. One ionic cross-linker (e.g., CaCl₂)) may be used to cross-link the internal shell from the inside-out, whereas another cross-linker (e.g., sodium tripolyphosphate (STP) may be used to cross-link the external shell from the inside out. As a further representative example, a core may be synthetic-based, an internal shell may be alginate-based, and an external shell may be PEGDA-based with a suitable photoinitiator such as Irgacure 2959 or LAP. One ionic cross-linker may be used to cross-link the internal shell from the inside-out, whereas UV or visible light may be used to partially cross-link the external shell. Such examples are meant to be representative and non-limiting.

### Radial Displacement

Embodiments herein pertain to annulus fibers comprised of a core, at least one annulus layer comprising at least one biological material surrounding the core, and at least one external and preferably immunoprotective shell layer which in turn surrounds the at least one annulus layer. In embodiments, the core is comprised of a reinforcing polymer, which provides mechanical strength to the entire fiber. In some examples, the polymer comprising the core is non-biodegradable, however in other examples the polymer comprising the core may comprise a biodegradable material, without departing from the scope of this disclosure.

In embodiments, the reinforcing polymer serves to radially displace the biological material embedded in the at least one annulus layer towards the periphery of the entire fiber structure. This positioning improves the access of the embedded cells to oxygen surrounding the fiber, and helps to maintain viability and/or function of the biological material. In embodiments, the core diameter range of annulus fibers of the present disclosure is between about 1.9 mm to 0.1 mm, more preferably between about 1.45 mm to 0.38 mm, and most preferably between about 0.98 mm to 0.45 mm. In some embodiments, the core diameter range of annulus fibers of the present disclosure is between about 1.92 mm to 0.05 mm, more preferably between about 1.1 mm to 0.41 mm, or between about 0.030 mm and about 1.0 mm, and most preferably between about 0.5 mm to 0.1 mm, for example about 0.3 mm.

In silico studies (refer to Examples 1, 2 and 3) evaluating the oxygen consumption rate of β-islet pancreatic cells have provided data on optimal core size to peripheralize the cells for access to diffusion by oxygen to maintain viability and function.

Surrounding the core, in annulus fiber embodiments, is the at least one annulus layer comprising a biocompatible hydrogel (e.g., alginate) that harbors the biological material(s). The biocompatible hydrogel material is selectively permeable to allow passage of select biologically active agents and materials (e.g., for a device used in the treatment of diabetes, such as type 1 or type 2 diabetes, passage of glucose, insulin, and oxygen) but which restricts the passage of the biological material(s) themselves (e.g., cells, exosomes, and the like). In embodiments, the thickness of the at least annulus layer is in the range of about 0.01 mm to 0.3 mm, more preferably between about 0.025 mm to 0.2 mm, and most preferably between about 0.05 mm to 0.125 mm. In some embodiments, the thickness of the annulus layer is between about 0.01 mm to 0.4 mm, more preferably between about 0.025 mm to 0.3 mm, more preferably between about 0.1 mm to about 0.3 mm, and most preferably between about 0.05 mm to 0.2 mm. In embodiments, the thickness is about 0.2 mm.

Surrounding the at least one annulus layer, in annulus fiber embodiments, is at least one external shell, preferably comprised of an immunoprotective material (e.g., zwitterionic modified alginate), which is also selectively permeable to allow passage of select agents and materials (e.g., passage of glucose, insulin, and oxygen for devices used in treatment of diabetes), but which restricts passage of immune cells, thus providing a physical barrier to T-cells, B-cells, and other immune cells, which may protect the encapsulated biological material from direct cell-mediated killing and implant rejection by the adaptive immune system. In embodiments, implant rejection via a fibrotic FBR can be reduced or avoided via use of specifically modified materials (e.g., functionalized alginates) in the at least one outer shell which reduces protein binding and, in turn, the subsequent binding and activation of macrophages, neutrophils, fibroblasts, and other cell types associated with the FBR (Qingsheng et al., Nature Communications. 2019; 10:5262).

Hence, as herein discussed, the at least one external shell layer (e.g., shells externally positioned compared to at least one annulus layer comprising biological material) serves as an immune-protective layer against the adaptive and innate immune response of the host, while still allowing the diffusion of oxygen, nutrients, and in embodiments, therapeutic agent(s) released by the encapsulated biological material(s). In embodiments, thickness of the at least one outer shell (e.g., total thickness in the case of more than one outer shell) may be in a range of about 50-200 μm. In some embodiments, the thickness may be between about 0.01 mm to 0.3 mm, or between about 0.015 mm and about 0.5 mm, more preferably between about 0.025 mm to 0.2 mm, or between about 0.05 mm to 0.125 mm. In embodiments, the thickness is about 0.150 mm. Thinner outer shells may be fragile and prone to degradation upon handling. In some embodiments, annulus fibers of the present disclosure have overall diameters of at least 1 mm. Implanted materials with size/diameters less than 1 mm are prone to elicitation of FBRs with corresponding development of fibrosis that encapsulates the implanted material (Watanabe et al., Biomaterials. 2020; 255:120162).

In some embodiments of an annulus fiber, as discussed herein, the at least one external shell layer comprises immunogenic materials and/or pro-vasculogenic materials. For example, an external shell layer surrounding the annulus layer may comprise both immunogenic materials and pro-vasculogenic materials. In embodiments, an annulus fiber may comprise an annulus layer comprising a biological material, where said annulus layer is surrounded by an immunoprotective shell layer, which is in turn surrounded by a pro-vasculogenic shell layer. In some embodiments, an annulus fiber may be surrounded by an immunoprotective layer and may not include a pro-vasculogenic layer. In some embodiments, an annulus fiber may be surrounded by a pro-vasculogenic layer and may not include an immunoprotective layer. Similar logic applies to core-shell fibers of the present disclosure, where biological material is included in the core (hence not radially displaced). For such a core-shell fiber, said core may be surrounded by an external shell comprising immunoprotective and/or pro-vasculogenic materials. In embodiments, said core of a core-shell fiber may be surrounded by an immunoprotective shell layer, and may not include a pro-vasculogenic layer. In embodiments said core of a core-shell fiber may be surrounded by a pro-vasculogenic shell layer, and may not include an immunoprotective layer.

As a representative example, there may be circumstances where it is desirable to include a pro-vasculogenic layer, but not necessarily an immunoprotective layer. Examples include fibers containing syngeneic biological materials (i.e., syngeneic cells), gene-modified stealth cells, or for use in patients who are on an immunosuppressive regimen.

### Segmentation/Compartmentalization

Embodiments herein also pertain to segmentation/compartmentalization of one or more layers (e.g., core and/or external layer in the case of core-shell fibers; core and/or inner shell layer and/or outer shell layer in the case of annulus fibers) of the tissue fibers of the present disclosure. In embodiments, one or more segments/compartments of a core and/or shell layer may be comprised of a biological material (e.g., cells).

Compartment sizing may be a function of one or more variables, including but not limited to tissue fiber size (e.g., length and/or diameter), type of fiber (e.g., core-shell fiber, annulus fiber), type of materials used in the process of tissue fiber generation, and the like. In some embodiments, a fiber may be comprised of at least two segments/compartments which include biological material, where other segments flanking the at least two segments/compartments are free of the biological material. For example, in the case of a core-shell fiber, at least two segments comprising biological material may be included within the core. In another example, in the case of an annulus fiber, at least two segments comprising biological material may be included within the annulus layer. In embodiments, segment(s) comprising biological material may be of greater length(s) than segment(s) lacking the biological material. In embodiments, segment(s) comprising biological material may be substantially similar in terms of length as compared to segment(s) lacking the biological material. In embodiments, segment(s) comprising the biological material for particular tissue fibers need not be the same length, but different segments may be comprised of different lengths. In embodiments, segment(s) lacking biological material for particular tissue fibers need not be the same length, but different segments may be comprised of different lengths. In some embodiments, spacing between compartments/segments inclusive of biological material (e.g., cells) in a tissue fiber of the present disclosure may be between 1-5 mm, for example 1 mm, 2 mm, 3 mm, 4 mm or 5 mm apart.

Segments/compartments comprised of biological material may comprise, for example, cells of particular densities. In embodiments, the density may be the same between compartments, or may be different. In embodiments, the biological material between compartments may be the same, or may be different. In embodiments, density of the biological material may be selected as a function of one or more of particular application (e.g., treatment of diabetes), cell viability determinants, material (e.g., biocompatible material) in which the biological material is included, and the like. As one example, density of a biological may comprise between 60,000-70,000 islet equivalents (IEQs) per ml, for example 65,000 IEQs/ml. Other biological material (e.g., hepatocytes) may be used in the tissue fibers of the present disclosure at similar, or different, densities.

In embodiments, one or more segments/compartments comprising biological material may be flanked by segments that comprise, for example, immunoprotective materials of the present disclosure. For example, in the case of a core-shell fiber in which the core includes two or more segments comprising biological material, the two or more segments may be flanked by other segments that comprise immunoprotective materials as herein disclosed. In other embodiments, the two or more segments comprising biological material may be flanked by other segments that do not comprise, for example, immunoprotective materials, without departing from the scope of this disclosure. Similar logic applies to annulus fibers of the present disclosure. For example, an annulus fiber may be comprised of two or more segments/compartments which are comprised of biological material, where each of the two or more segments/compartments may be flanked by segments that incorporate, for example, immunoprotective materials of the present disclosure. In other embodiments, the two or more segments comprising biological material may be flanked by segments that do not comprise for example, immunoprotective materials, without departing from the scope of this disclosure.

### Coating

Within the scope of this disclosure is the coating of a bioprinted fiber with a material that imparts one or more desired properties to the bioprinted fiber. Coating can thus be used to add an outermost external shell layer to a bioprinted fiber. A bioprinted fiber may be coated with a coating material by way of submerging a bioprinted fiber in the coating material, or otherwise applying the coating material to a bioprinted fiber (e.g., spraying, dispensing the coating material onto the bioprinted fiber by way of a print head or other dispensing means (i.e., pump), etc.). As a representative example, a bioprinted fiber may comprise an annulus fiber comprised of a core, an annulus layer, and an immunoprotective external shell layer. A pro-vasculogenic layer may be added by way of coating, for example the bioprinted fiber may be coated with a pro-vasculogenic material such as polymethacrylic acid or other polymeric material. It is to be understood that addition of a coating layer in the manner described is not limited to pro-vasculogenic layers, but can include, for example, an outermost layer comprised of both pro-vasculogenic material and immunoprotective material, or just immunoprotective material.

All patents and patent publications referred to herein are hereby incorporated by reference in their entirety.

### EXAMPLES

### Example 1: Design of Bioprinted Fibers with Control Over Radial Placement of Encapsulated Cells

This Example pertains to bioprinting of multi-layered alginate fibers as macroencapsulation devices where a cell-containing layer is constricted between two acellular layers, i.e., radially displaced to form an annulus. Geometries for these multi-layered alginate fibers were determined based on oxygen transfer modeling and compared to bi-layered fibers without radial displacement of the cell-containing layer. Sensitivity analyses were performed to examine the effects of variations in model parameters and fiber dimensions on the oxygen profiles of encapsulated cells and their insulin secretion rates. As shown, a distinguishing attribute of multi-layered alginate fibers with radially displaced cell layers was their increased cell loading capacity while preventing formation of cell layers with submaximal insulin secretion and/or anoxic conditions.

As discussed in this Example, we extended analytical modeling of oxygen transfer to analyze cylindrical cell (e.g., islet) encapsulation devices comprised of three alginate layer fibers, where radial displacement of the cell layer resulted in an annular cell region. Our theoretical model combined estimation of fractional insulin secretion rate, in the presence and absence of oxygen limitations, with elements from two previously published analytical solutions for oxygen consumption and diffusion in cylindrical cell encapsulation devices. Their cell loading per encapsulation volume and insulin secretion capacities were compared with two-layer cylindrical geometries. A sensitivity analysis of the parameter values that vary between cell types or are uncertain was performed in order to provide information on the impacts of this variability on the modeled designs of the three-layer fibers. A second sensitivity analysis of the design parameters for three-layer fibers was conducted in order to provide information on the impacts of process variability on cell oxygenation and secretory capacity.

### Oxygen Transfer Model

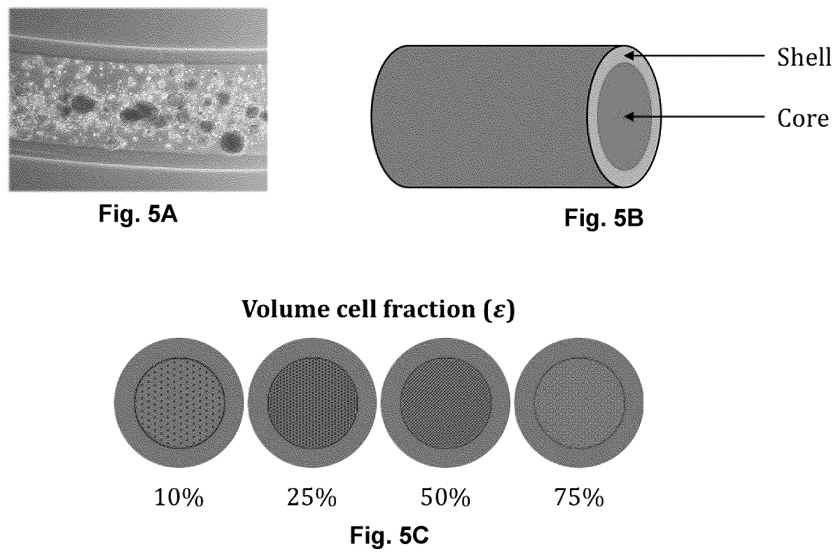

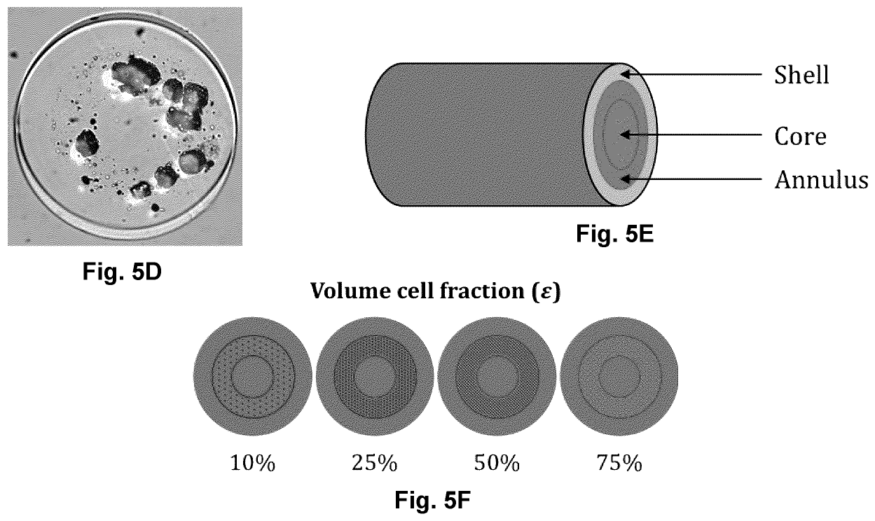

Discussed in this Example, we extended analytical modeling of oxygen transfer to analyze cylindrical cell encapsulation devices comprised of three alginate layer fibers. As noted above, we combined elements from the oxygen mass transfer models presented by Avgoustiniatos and Colton, Ann N Y Acad Sci. 1997; 831(1):145-67 and Iwata et al., Artif Organs. 2018; 42(8):E168-85. Both consider steady-state diffusion and zero-order consumption of oxygen by encapsulated cells. The former analyzed encapsulated cell oxygenation after the formation of fibrotic or vascular cell layers on the surface of the encapsulation device, and explored the formation of necrotic cell layers due to oxygen limitations Avgoustiniatos and Colton, Ann N Y Acad Sci. 1997; 831(1):145-67. Conversely, the latter model focused on fully oxygenated inner cell layers surrounded by immunoprotective membranes Iwata et al., Artif Organs. 2018; 42(8):E168-85. Here, we model fibers composed of either two or three layers without extracapsular cell overgrowth, where the cells are homogeneously distributed throughout an inner layer with average cell volume fraction E (FIGS. 5A-5F). This assumption was present in Avgoustiniatos' model but absent in Iwata's. Therefore, the cross-section of the two-layer fiber can be represented by a core cell region with average cell volume fraction E, surrounded by a shell of encapsulant (FIGS. 5A-5C). Similarly, for the three-layer fiber with cells homogenously distributed in its middle layer, the fiber cross-section can be represented by an annular cell region around an encapsulant core both enclosed by a shell of encapsulant (FIGS. 5D-5F). For both fiber cross-sections, the oxygen consumption rate per unit volume of cell region is

$${q}_{{O}_{2}} = {V}_{max} ε \qquad ( 1 )$$

where Vₘₐₓ denotes the maximum oxygen consumption rate of the cells per unit volume.

As noted above, zero-order kinetics of oxygen consumption were also assumed by Avgoustiniatos and Iwata in order to solve the oxygen mass transfer equations analytically (Avgoustiniatos and Colton, Ann N Y Acad Sci. 1997; 831(1):145-67; Iwata et al., Artif Organs. 2018; 42(8):E168-85). Also, using the finite difference method, Avgoustiniatos later reported a slight increase in oxygenated cell layer thickness predicted with Michaelis-Menten kinetics in cylindrical geometry, but warned that this sliver of cells is exposed to extremely low oxygen concentrations and it is unlikely to contribute much to insulin secretion capacity (Avgoustiniatos E S, Massachusetts Institute of Technology; 2001).

In the model presented by Avgoustiniatos (Avgoustiniatos and Colton, Ann N Y Acad Sci. 1997; 831(1):145-67), effective diffusion coefficients of oxygen αD (in mol·cm⁻³·mmHg⁻¹·s⁻¹) are obtained for each material from the multiplication of the oxygen solubility coefficient α and the diffusion coefficients of oxygen D. This incorporates conversion of oxygen concentrations to partial pressures and simplifies boundary conditions at interfaces by circumventing the need to use partition coefficients (Avgoustiniatos E S, Massachusetts Institute of Technology; 2001). The effective diffusion coefficient of oxygen for fiber layer k, comprised of dispersed (d) and continuous phases (c) (e.g., encapsulant made up of alginate in water, cell region made up of cells in encapsulant), is calculated using Maxwell's relationship for composite media:

$$\frac{{( α D )}_{k}}{{( α D )}_{c}} = \frac{2 - 2 ϕ + ρ ( 1 + 2 ϕ )}{2 + ϕ + ρ ( 1 - ϕ )} \qquad ( 2 )$$

where ϕ denotes the volume fraction of the dispersed phase, and ρ=(αD)_{d}/(αD)_{c} the ratio of the modified effective diffusion coefficients between the dispersed and continuous phases.

From here on, we follow the analytical solution presented by Iwata for cylindrical geometry (Iwata et al., Artif Organs. 2018; 42(8):E168-85) but fiber layers are labeled inward and include Rₘᵢₙ, which serves as a dummy variable in the absence of oxygen limitations. Thus, Layer 1 corresponds to the encapsulant shell, Layer 2 to the cell region, and Layer 3 to the encapsulant core (if present). Also, the two-layer and three-layer fibers are called core-shell and core-annulus-shell fibers, respectively.

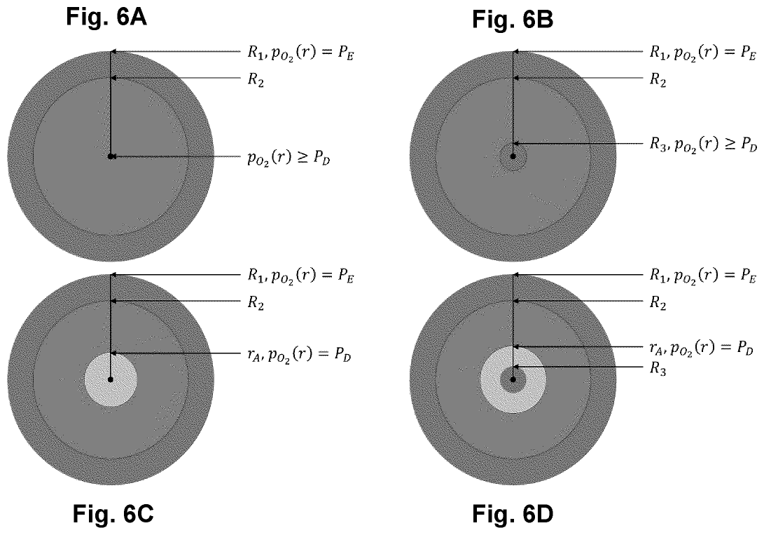

Radial diffusion and constant oxygen partial P_{E} around the fiber are assumed to enable analytical solutions for the species conservation equation. Given these conditions, the amount of oxygen passing through the encapsulant shell is balanced with the oxygen consumed by the cell region:

$$2 π {r ( α D )}_{1} \frac{\partial {p}_{{O}_{2}} ( r )}{\partial r} = {q}_{{O}_{2}} π ( {R}_{2}^{2} - {R}_{min}^{2} ) \qquad ( 3 )$$

where Rₘᵢₙ=0 for a core-shell fiber (FIG. 6A) and Rₘᵢₙ=R₃ for a core-annulus-shell fiber (FIG. 6B) in the absence of oxygen limitations. Conversely, Rₘᵢₙ=r_{A} for both fibers in the presence of anoxia, where r_{A} is the boundary of the anoxic layer in the cell region in terms of radial distance (FIG. 6C, 6D). Since r_{A} is typically unknown, its estimation is described in section “Sensitivity analysis of modeled parameters” below. The oxygen partial pressure at the surface of the shell is equal to P_{E}. Inserting this boundary condition in equation Error! Reference source not found. yields the oxygen partial pressure profile in the shell (R₂≤r≤R₁)

$${p}_{{O}_{2}} ( r ) = {P}_{E} - \frac{{q}_{{O}_{2}} ( {R}_{2}^{2} - {R}_{min}^{2} )}{2 {( α D )}_{1}} ln ( \frac{{R}_{1}}{r} ) \qquad ( 4 )$$

and the oxygen partial pressure at the interface between the shell and the cell region p_{O}_{₂}(R₂)

$${p}_{{O}_{2}} ( {R}_{2} ) = {P}_{E} - \frac{{q}_{{O}_{2}} ( {R}_{2}^{2} - {R}_{min}^{2} )}{2 {( α D )}_{1}} ln ( \frac{{R}_{1}}{{R}_{2}} ) \qquad ( 5 )$$

In the cell region, the amount of oxygen delivered and being consumed are also balanced:

$$2 π {r ( α D )}_{2} \frac{\partial {p}_{{o}_{2}} ( r )}{\partial r} = {q}_{{o}_{2}} π ( {r}^{2} - {R}_{min}^{2} ) \qquad ( 6 )$$

The oxygen partial pressure at the shell-cell region interface p_{O}_{₂}(R₂) can be obtained from equation Error! Reference source not found., and the oxygen partial pressure profile in the cell region (Rₘᵢₙ≤r≤R₂) is:

$${p}_{{O}_{2}} ( r ) = {P}_{E} - \frac{{q}_{{O}_{2}} ( {R}_{2}^{2} - {R}_{min}^{2} )}{2 {( α D )}_{1}} ln ( \frac{{R}_{1}}{{R}_{2}} ) - \frac{{q}_{{O}_{2}} {R}_{2}^{2}}{4 {( α D )}_{2}} [ 1 - {( \frac{r}{{R}_{2}} )}^{2} - 2 {( \frac{{R}_{min}}{{R}_{2}} )}^{2} ln ( \frac{{R}_{2}}{r} ) ] \qquad ( 7 )$$

### Absence or Presence of Anoxia in the Cell Region

The model presented by Avgoustiniatos (Avgoustiniatos and Colton, Ann N Y Acad Sci. 1997; 831(1):145-67) assumes there is a critical oxygen partial pressure P_{D} at which cells become anoxic and die, a cell region without anoxia has p_{O}_{₂}(Rₘᵢₙ)≥P_{D}. Inserting this boundary condition into equation Error! Reference source not found.:

$${P}_{D} \leq {P}_{E} - \frac{{q}_{{O}_{2}} ( {R}_{2}^{2} - {R}_{min}^{2} )}{2 {( α D )}_{1}} ln ( \frac{{R}_{1}}{{R}_{2}} ) - \frac{{q}_{{O}_{2}} {R}_{2}^{2}}{4 {( α D )}_{2}} [ 1 - {( \frac{{R}_{min}}{{R}_{2}} )}^{2} - 2 {( \frac{{R}_{min}}{{R}_{2}} )}^{2} ln ( \frac{{R}_{2}}{{R}_{min}} ) ] \qquad ( 8 )$$

where Rₘᵢₙ=0 for a core-shell fiber and Rₘᵢₙ=R₃ for a core-annulus-shell fiber. In a cell region with anoxia, p_{O}_{₂}(Rₘᵢₙ)=P_{D} and Rₘᵢₙ=r_{A}, where r_{A}>0 for a core-shell fiber and r_{A}>R₃ for a core-annulus-shell fiber. Inserting this boundary condition into equation Error! Reference source not found.:

$${P}_{D} = {P}_{E} - \frac{{q}_{{O}_{2}} ( {R}_{2}^{2} - {r}_{A}^{2} )}{2 {( α D )}_{1}} ln ( \frac{{R}_{1}}{{R}_{2}} ) - \frac{{q}_{{O}_{2}} {R}_{2}^{2}}{4 {( α D )}_{2}} [ 1 - {( \frac{{r}_{A}}{{R}_{2}} )}^{2} - 2 {( \frac{{r}_{A}}{{R}_{2}} )}^{2} ln ( \frac{{R}_{2}}{{r}_{A}} ) ] \qquad ( 9 )$$

Equation Error! Reference source not found. can be solved iteratively to find a set of fiber dimensions (denoted by R₁, R₂ and R₃) or cell volume fraction (embedded in (αD)₁ and q_{O}_{₂}) that ensure a fully oxygenated cell region. When the right-hand side of equation Error! Reference source not found. yields negative values, equation Error! Reference source not found. should be used to estimate r_{A} through iterations.

Finally, the cell loading per unit length of fiber is:

$$Cell loading = \frac{( {R}_{2}^{2} - {R}_{min}^{2} ) ε}{{R}_{1}^{2}} \qquad ( 10 )$$

Where Rₘᵢₙ=0 in the core-shell fiber and Rₘᵢₙ=R₃ in the core-annulus-shell fiber. In the presence of anoxia, cell loadings for both fiber geometries can be calculated only for fully oxygenated areas in the cell regions by substituting Rₘ=r_{A}.

### Insulin Secretion Rate Model

A bilinear model of insulin secretion rate as a function local oxygen partial pressure (Avgoustiniatos E S, Massachusetts Institute of Technology; 2001; Johnson et al., Chem Eng Sci. 2009; 64(22):4470-87) is used to estimate the net insulin secretion rate per unit length of fiber.

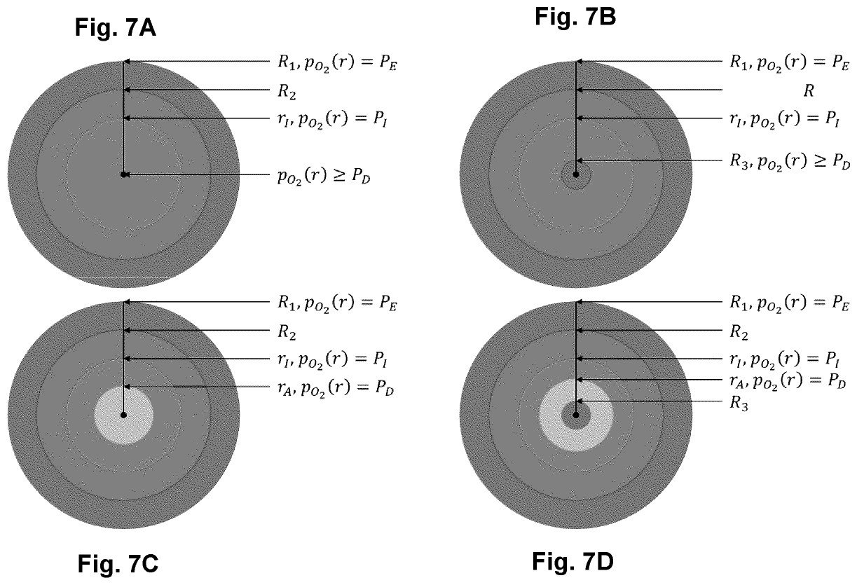

The first model assumption denotes P_{I}, a local oxygen partial pressure below which cells secrete insulin at a lower rate. The resulting cell layer with submaximal insulin secretion rate has a radial boundary r_{I} and, since P_{I}>P_{D}, it follows that r_{I}≥Rₘᵢₙ for both types of fibers even if there is anoxia. Inserting this boundary condition into equation Error! Reference source not found.:

$${P}_{I} = {P}_{E} - \frac{{q}_{{O}_{2}} ( {R}_{2}^{2} - {R}_{min}^{2} )}{2 {( α D )}_{1}} ln ( \frac{{R}_{1}}{{R}_{2}} ) - \frac{{q}_{{O}_{2}} {R}_{2}^{2}}{4 {( α D )}_{2}} [ 1 - {( \frac{{r}_{I}}{{R}_{2}} )}^{2} - 2 {( \frac{{R}_{min}}{{R}_{2}} )}^{2} ln ( \frac{{R}_{2}}{{r}_{I}} ) ] \qquad ( 11 )$$

where Rₘᵢₙ=0 in a core-shell fiber (FIG. 7A) and Rₘᵢₙ=R₃ in a core-annulus-shell fiber (FIG. 7B) with fully oxygenated cell regions; while in the presence of anoxia, Rₘᵢₙ=r_{A} for both fibers (FIGS. 7C, 7D). When the right-hand side of this equation yields values greater than P_{I}, this means that r_{I}=Rₘᵢₙ and there is no cell layer with submaximal insulin secretion rate.

The second model assumption specifies that for p_{O}_{₂}(r)<P_{I}:

$$f ( r ) = \frac{{p}_{{O}_{2}} ( r )}{{P}_{I}} \qquad ( 12 )$$

and for p_{O}_{₂} (r)≥P_{I}:

$$f ( r ) = 1 \qquad ( 13 )$$

where f is the fraction of maximum insulin secretion rate. Therefore, the cross-sectional area of the cell layer with maximum insulin secretion rate:

$${A}_{M} = π ( {R}_{2}^{2} - {r}_{I}^{2} ) \qquad ( 14 )$$

While the net cross-sectional area of the cell layer with submaximal insulin secretion rate:

$${A}_{S} = {\int }_{{R}_{m}}^{{r}_{I}} f ( r ) 2 π rdr \qquad ( 15 )$$

where Rₘᵢₙ=0 in the core-shell fiber and Rₘᵢₙ=R₃ in the core-annulus-shell fiber with fully oxygenated cell regions; while in the presence of anoxia, Rₘᵢₙ=r_{A} for both fibers. Finally, the secretory capacity per unit length of fiber is:

$$Secretory capacity = \frac{( {A}_{N} + {A}_{S} ) ε}{π {R}_{1}^{2}} \qquad ( 16 )$$

### Model Implementation

Calculations of oxygen partial pressure and fraction of insulin secretion rate as a function of radial distance were carried out in MS Excel. Goal Seek was used to solve equation Error! Reference source not found. for ε when given a target p_{O}_{₂}(Rₘᵢₙ), set to P_{D} or P_{I}, and constant fiber dimensions, where fiber diameter was 2R₁, shell thickness R₁-R₂, annulus thickness R₂-R₃ (if applicable), and core diameter 2R₂ or 2R₃. Alternatively, when the right-hand size of equation Error! Reference source not found. yielded negative values, the same p_{O}_{₂}(Rₘᵢₙ) target, fiber dimensions and E were used in equation Error! Reference source not found. to find r_{A} via Goal Seek. Then, a separate Goal Seek estimated r_{I} from equation Error! Reference source not found. Visual Basic scripts were used to automate iterative calculations and the integration of equation Error! Reference source not found.

### Model Parameter Selection

Values for model parameters were retrieved from the literature (see Table 2). Despite encountering variability in some parameter values, a majority of these were obtained from the modelling work carried out by Avgoustiniatos, Colton and others (Avgoustiniatos E S, Massachusetts Institute of Technology; 2001; Johnson et al., Chem Eng Sci. 2009; 64(22):4470-87). However, we chose to estimate Vₘₐₓ from measurements of human islet oxygen consumption rate reported by Pappas and colleagues (Papas et at, PLoS One. 2015; 10(8):e0134428), and measurements of human islet cellular composition (Pisania et al., Lab Investig. 2010; 90(11):1661-75; Pisania et al., Lab Investig. 2010; 90(11):1676-86). To address variability in parameter values, a one factor at a time sensitivity analysis was conducted where the range of changes in input variables covered those found reported in the literature (see section entitled “Sensitivity analysis of modeled parameters” below).

TABLE 2
Parameters for oxygen transfer model

| Parameter | Symbol | Value | Units |
|---|---|---|---|
| Oxygen partial | P_{E} | 40 | mmHg |
| pressure in peritonetal | | | |
| cavity (external to | | | |
| implant) | | | |
| Minimum oxygen | P_{I} | 5.1 | mmHg |
| partial pressure at ells | | | |
| for maximum insulin | | | |
| secretion | | | |
| Critical oxygen partial | P_{D} | 0.1 | mmHg |
| pressure at which cells | | | |
| die | | | |
| Maximum oxygen | Vₘₐₓ | 1.4 · 10⁻⁸ | mol · cm⁻³ · s⁻¹ |
| consumption rate of | | | |
| iselts per unit | | | |
| volume ^{A} | | | |
| Effective diffusion | (αD)_{water} | 3.5 · 10⁻¹⁴ | mol · cm⁻¹ · |
| coefficient of oxygen | | | mmHg⁻¹ · s⁻¹ |
| in water ^{B} | | | |
| Effective diffusion | (αD)_{cells} | 1.2 · 10⁻¹⁴ | mol · cm⁻¹ · |
| coefficient of oxygen | | | mmHg⁻¹ · s⁻¹ |
| in cells ^{B} | | | |
| Effective diffusion | (αD)_{alginate} | 0 | mol · cm⁻¹ · |
| coefficient of oxygen | | | mmHg⁻¹ · s⁻¹ |
| in alginate ^{B} | | | |

^{A} Converted from mean oxygen consumption rate per DNA, assuming 1500 cells per islet and 6.9 pg of DNA per cell.
^{B} Modified effective diffusion coefficients of oxygen in pure components used in equation 2.

### Results

### Comparison of Core-Shell and Core-Annulus-Shell Geometries

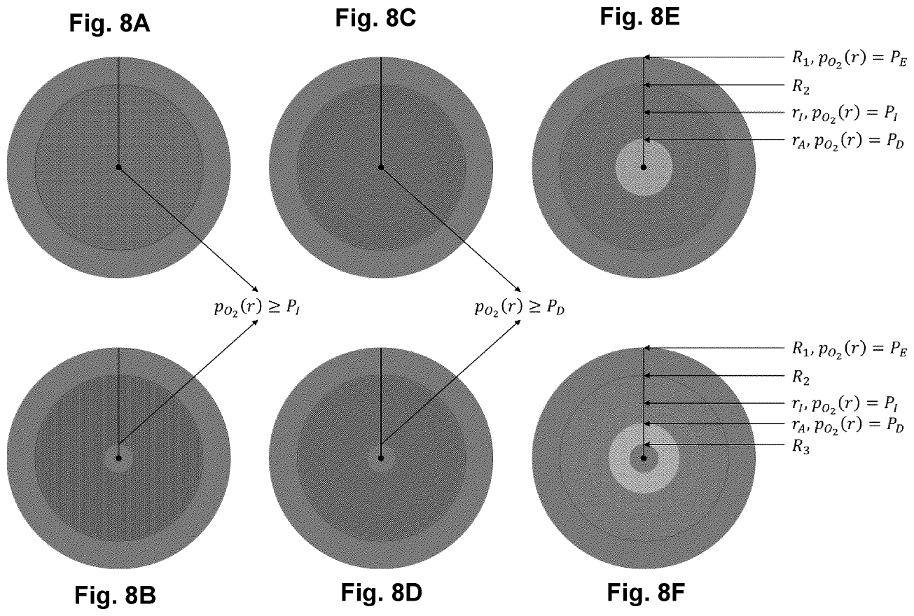

In co-axial flow-focusing fiber printing, dimensions can be adjusted by manipulation of flow rates, material viscosities, as well as print head design. We focused on 1 mm fibers because features (e.g., microspheres) of 1-1.5 mm have been shown to have reduced FBR as compared to smaller microspheres (Veiseh et al., Nat Mater. 2015 June; 14(6):643-51). Oxygen depletion was analyzed to compare core-shell and core-annulus-shell fibers, both with a 150 μm immunoprotective shell consisting of 4% alginate and a cell region with 2% alginate. Simulations were performed for these fibers exposed to a 40 mmHg intraperitoneal oxygen partial pressure and cell volume fractions (E) were varied to identify the maximum cell loading at which cell regions were fully oxygenated or had maximal insulin secretion rate (FIG. 8A-8D). Increasing cell volume fraction past the former led to oxygen limitations (FIG. 8E-8F), and thus we explored this case only for a core-shell fiber when matching its cell loading to that of core-annulus-shell fibers (see below).

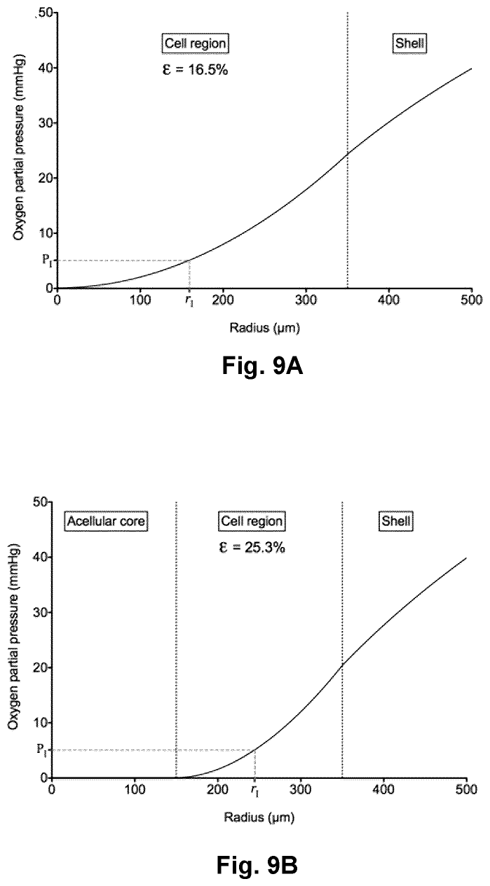

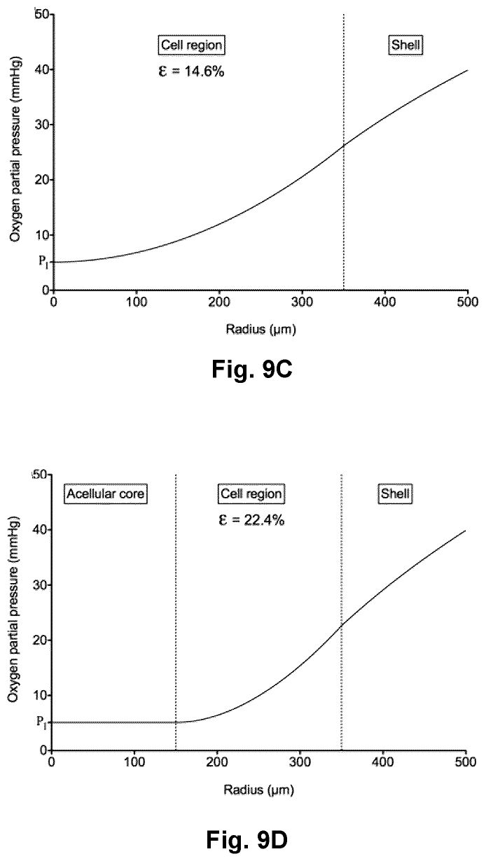

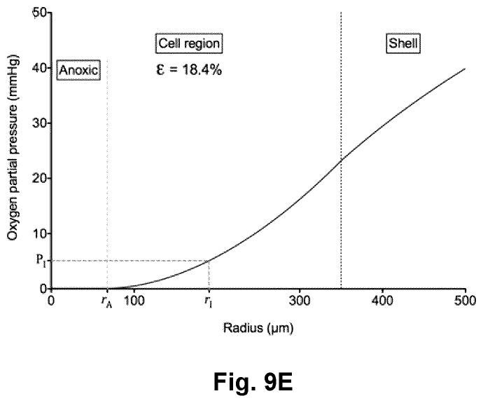

Maximum cell volume fractions were first calculated for fully oxygenated cell regions, i.e., simulations targeted a minimum oxygen concentration of 0.1 mmHg. As shown in FIG. 9A, for the core-shell fiber cell region to be fully oxygenated the maximum E was 16.5%. This included an inner cell layer with submaximal insulin secretion rates spanning 159 μm of the 350-μm cell region radius (schematized as the orange region in FIG. 8C). Adding a 150 μm radius acellular core and thereby a 200-μm thick annulus of cells, in the core-annulus-shell geometry the maximum E was increased to 25.3%, and the cell layer with submaximal insulin secretion rate was only 94 μm in thickness (FIG. 9B). For all of the cells to be producing insulin at the maximum rate and thereby maximize their utility, additional simulations targeted a minimum oxygen concentration of 5.1 mmHg (Table 2). This lowered the maximum E of the core-shell fiber from 16.5% to 14.6% (FIG. 9C), and of the core-annulus-shell fiber from 25.4% to 22.4% (FIG. 9D). Increasing the cell volume fraction of the core-shell fiber from 16.5% to 18.4% resulted in an anoxic cell layer spanning 68 μm of the cell region radius, and enveloped by a 123-μm layer of cells with submaximal insulin secretion rate (FIG. 9E).

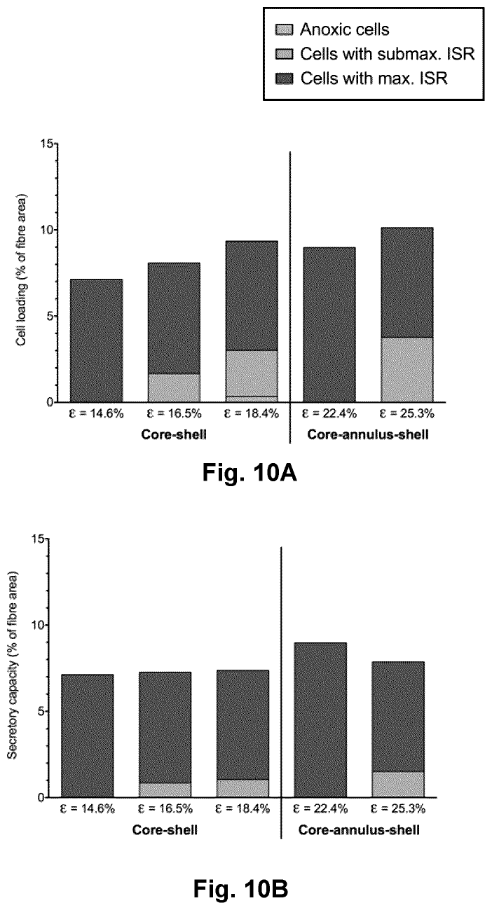

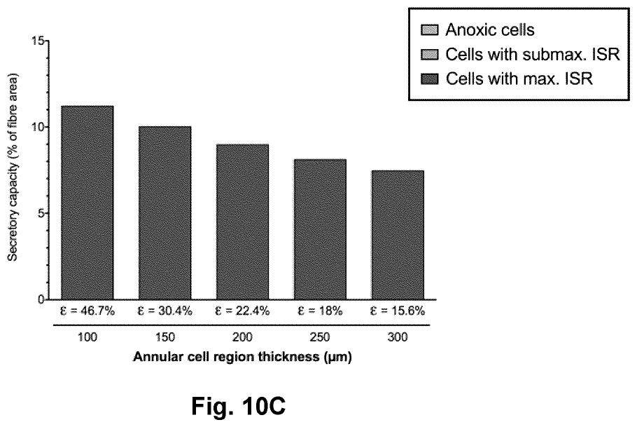

The acellular core in the core-annulus-shell fibers restricted the cell region area to 40% of the fiber area, compared to 49% in core-shell fibers. However, the radial displacement of the cell region essentially placed the cells closer to the oxygen source than fibers with a core-shell geometry, such that both the cell volume fraction and the proportion of cells at maximal insulin secretion was increased. For example, in the fibers with maximum insulin secretion rate where minimum oxygen concentration targeted 5.1 mmHg, the cell loadings (see equation 10) were 7.10% of fiber area for the core-shell, and 9% for the core-annulus-shell geometries (FIG. 10A). Even though this might seem a marginal increase, matching the latter cell loading in a core-shell fiber with a 350-μm radius cell region required increasing E to 18.4%, but this resulted in 3.7% of the cell region area becoming anoxic and 25.9% having submaximal insulin secretion. Both of these cell layers could release of damage-associated molecular patterns (DAMPs) in vivo and elicit graft failure (Paredes-Juarez et al., Sci Rep. 2015; 5(September):1-12).

Moreover, as shown in FIG. 10B, the secretory capacity of this core-shell fiber was only 7.4% of fiber area, whereas the same was 9% for the core-annulus-shell fiber with maximum insulin secretion (i.e., equal to its cell loading). For core-shell fibers with a fully oxygenated cell region, secretory capacity was 7.3% of fiber area. Overall, the annular cell region with maximum insulin secretion rate increased secretory capacity by at least 20% when compared to core-shell fibers. Interestingly, the core-annulus-shell fiber with a fully oxygenated cell region had a cell loading of 10.10% (FIG. 10A), higher than its counterpart with maximum insulin secretion rate, but its secretory capacity was only 7.9% of fiber area (FIG. 10B) because the inner 37% of the cell region area was exposed to oxygen concentrations below 5.1 mmHg (FIG. 9B). The 14% decrease in secretory capacity highlighted the importance of targeting maximal insulin secretion for annular cell regions rather than aiming to encapsulate the maximum number of cells that can be oxygenated.

For the 1 mm fibers with 150 μm immunoprotective shells, increasing cell volume fraction led to minute increases in core-shell fiber secretory capacity. Conversely, the core-annulus-shell fiber with higher cell volume fraction had lower secretory capacity. However, the geometry of these fibers enabled modulation of annulus thickness while fiber radius and shell thickness were kept constant. Therefore, we explored the effects of varying cell region annulus thickness on secretory capacity. In these simulations, cell volume fractions (E) were adjusted to identify the maximum cell loading at which cell regions had maximal insulin secretion rate. As shown in FIG. 10C, the maximum E and secretory capacity both decreased as the thickness of the annulus cell region was increased. In other words, reducing the radial displacement of the cell region essentially placed the cells farther away from the oxygen source, and approximated core-shell fibers. On the other hand, fibers with thinner annular cell regions, besides increased secretory capacities, could potentially minimize the occurrence of radially stacked cell aggregates competing for oxygen diffusing inwards. The dimensions and size distribution of insulin-producing cell aggregates (Tse et al., Front Bioeng Biotechnol. 2021; 9:634403; Huang et al., Cell Transplant. 2018 Jul. 28; 27(7):1017-26; Schulz et al., PLoS One. 2012 January; 7(5):e37004; Rezania et al., Nat Biotechnol. 2014; 32(11):1121-33; Pagliuca et al., Cell. 2014; 159(2):428-39), such as islets, pose a lower bound on minimum the annulus thickness for encapsulation. However, smaller re-aggregated islets (Kojima et al., Organogenesis. 2014; 10(2):225-30) could be used to enable the use and leverage the benefits of further reductions in annulus cell region dimensions.

### Sensitivity Analysis for Core-Annulus-Shell Fibers

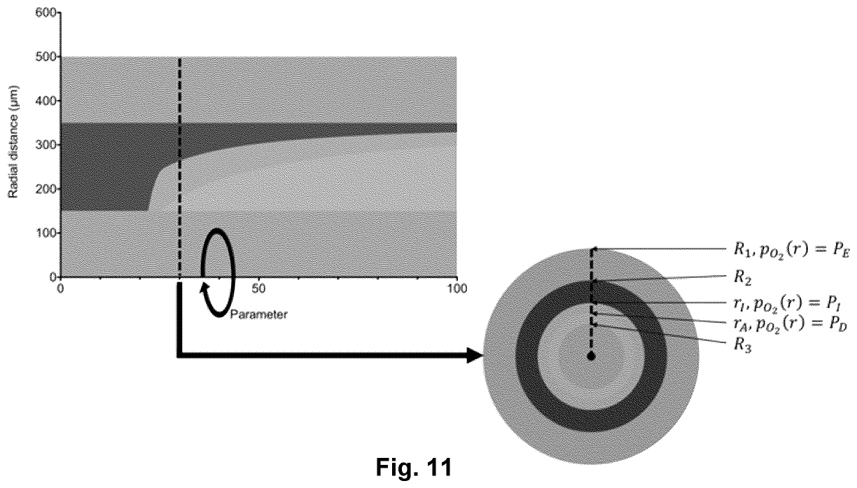

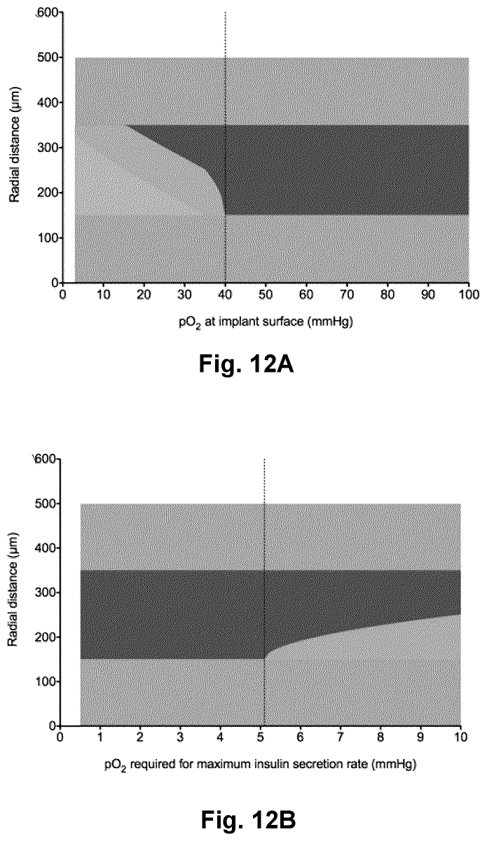

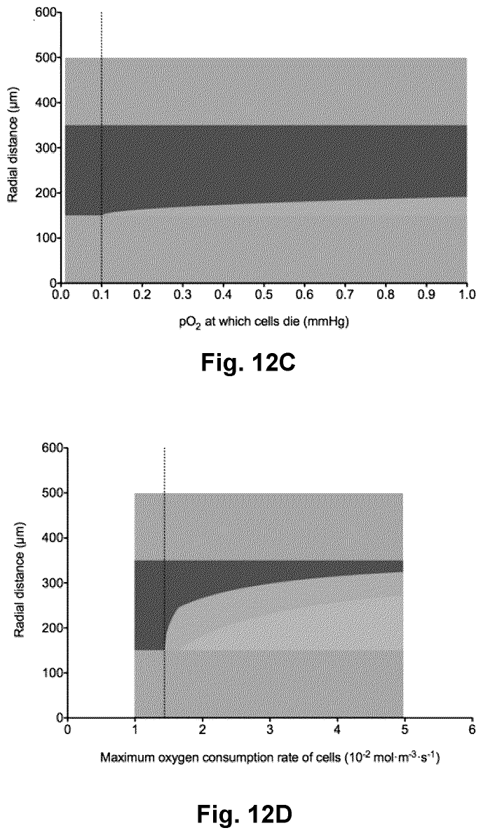

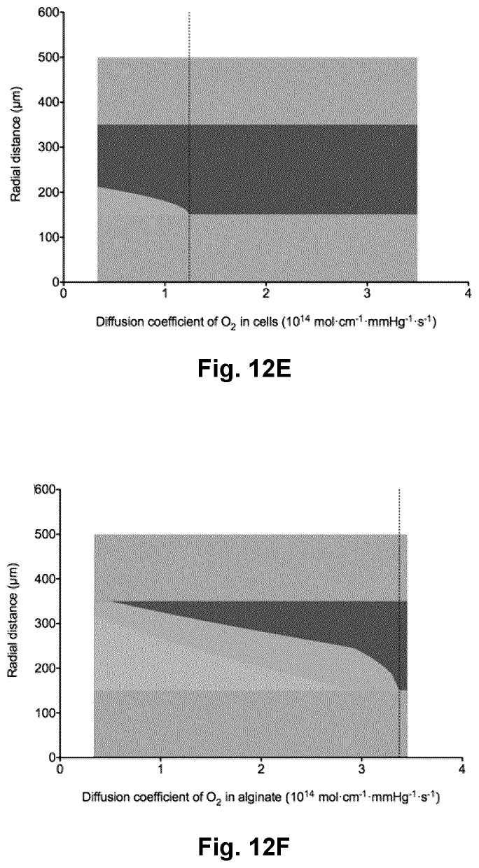

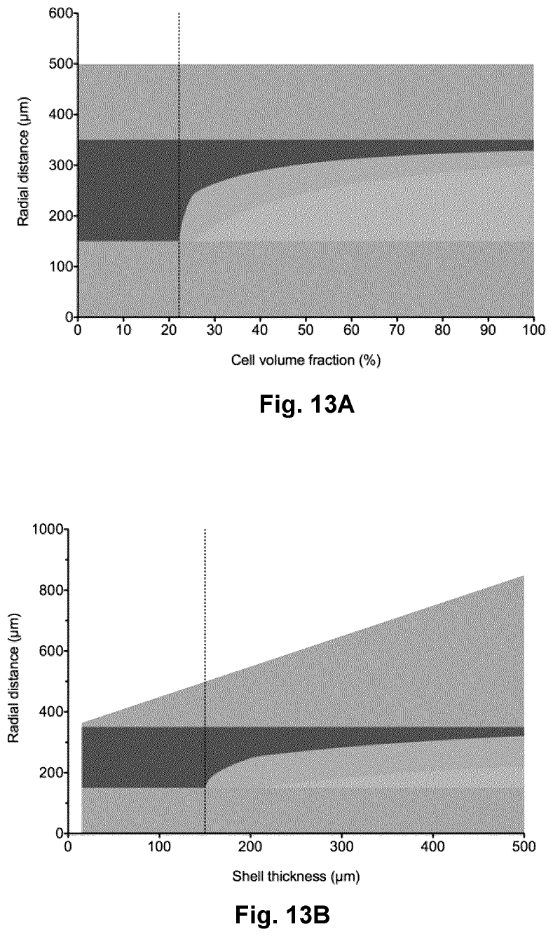

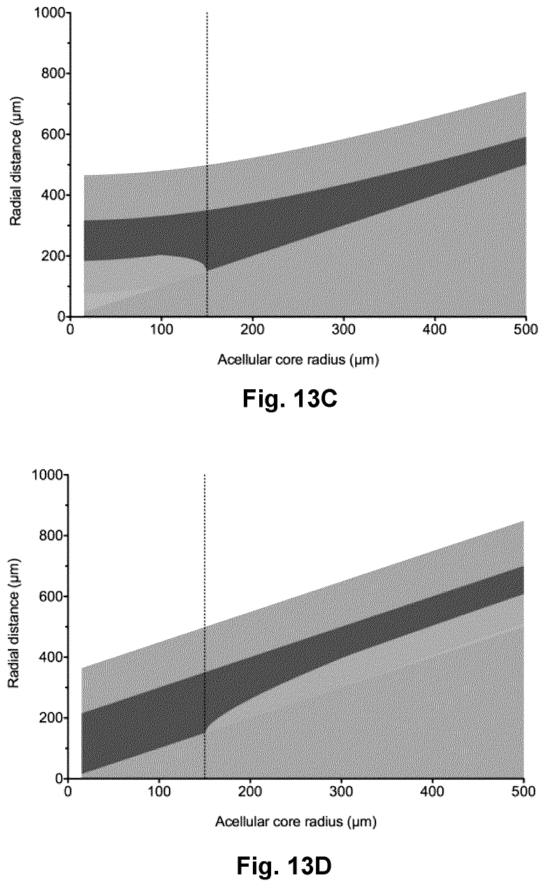

Mathematical models provide useful predictions despite the uncertainty as well as true variability of the model parameter values such as those used in the above analysis. A model is even more useful if it can explore the consequences of this variability, and this was done by performing a one factor at a time sensitivity analysis to explore variations in the model parameters shown in Table 2 (FIGS. 12A-12F). A similar analysis was performed while varying fiber parameters (cell volume fraction, shell thickness, annulus thickness and acellular core diameter) to assess a range of different fiber designs and/or variations that might occur while printing (FIGS. 13A-13D). In both sensitivity analyses, the core-annulus-shell fiber in FIG. 9D was selected as the base case, since it was predicted to have the highest cell loading while maintaining the maximum insulin secretion rates of all the implanted cells. As shown in FIG. 11, results of these analyses were summarized as the radial boundaries of different fiber layers, which can be rotated perpendicularly around the x-axis to form a fiber cross-section.

### Sensitivity Analysis of Model Parameters

In the first sensitivity analysis, we started by addressing the uncertainty in the partial oxygen pressure at the surface of the fiber (P_{E}). Various oxygen concentrations have been reported and/or measured intraperitionally (and other implantation sites) in different studies in animals, non-human primates and humans (Safley et al., Transplantation. 2020; 104(2):259-69; Papas et al., Adv Drug Deliv Rev. 2019; 139:139-56. Thus, the analyzed range for P_{E} was 3-100 mmHg, and included oxygen partial pressures found in blood. As described earlier, the analyzed core-annulus-fiber had maximum insulin secretion throughout the cell region when exposed to surface oxygen partial pressure P_{E}=40 mmHg. Thus, increasing P_{E} past this base case value led to a concomitant rise in the minimum oxygen partial pressure encountered in the fiber cross-sections. Conversely, as seen in FIG. 12A, even moderate decreases in P_{E} resulted in the formation and rapid thickening of the cell layer with submaximal insulin secretion rate. Expansion of this cell layer was reduced with anoxic layer formation at P_{E}≤34 mmHg. Maximum insulin secretion rate could not be sustained at P_{E}≤15 mmHg.

Next, we looked at the uncertainty in the oxygen partial pressures required for maximum insulin secretion rate (P_{I}) and at which cells die (P_{D}). The value of P_{I} was estimated by Avgoustiniatos (Avgoustiniatos E S, Massachusetts Institute of Technology; 2001) from insulin secretion measurements of canine and murine islets exposed to different bulk oxygen concentrations (Dionne et al., Diabetes. 1993; 42(1):12-21). However, the value of P_{I} might be different for human islets and, to the best of our knowledge, this pressure has not been measured directly. Therefore, the analyzed range for P_{I} was 0.5-10 mmHg, where the lower bound approximated the Michaelis-Menten constant of pancreatic cells for oxygen (Dionne et al., Diabetes. 1993; 42(1):12-21) and the upper bound was previously used by Avgoustiniatos in the development of the fractional insulin secretion rate model (Avgoustiniatos E S, Massachusetts Institute of Technology; 2001). As for the value of P_{D}, it was determined as the critical oxygen partial pressure for induction of hypoxic cell death in hepatocytes (Anundi and Groot, Am J Physiol. 1989 July; 257(1 Pt 1):G58-64. Since this study is cited by most cell encapsulation oxygen transfer models, we resorted to using a ten-fold decrease/increase (0.01-1 mmHg) as the analyzed range for P_{D}. Changes in the values of P_{I} and P_{D} did not impact the minimum oxygen partial pressure encountered in the fiber cross-sections. However, gradually thicker cell layers with submaximal insulin secretion rate but no anoxic cell layers were observed at P_{I}>5.1 mmHg (FIG. 12B). This pattern was also observed for P_{D}>0.1 mmHg, but with a noticeably slower growth of the cell layers with submaximal insulin secretion rate (FIG. 12C).

The oxygen consumption rate of human islets and other insulin-producing cells was recently summarized by Tse and others (Tse et al., Front Bioeng Biotechnol. 2021; 9: 634403. Therefore, we used these values (0.01-0.05 mol·m³·s⁻¹) as the analyzed range for the maximum oxygen consumption rate of cells (Vₘₐₓ). Variations in the value of Vₘₐₓ were proportionally inverse with the minimum oxygen partial pressure encountered in the fiber cross-sections. Therefore, for Vₘₐₓ>0.014 mol·m⁻³ s⁻¹ (the base case value) there was a rapid thickening of the cell layer with submaximal insulin secretion rate (FIG. 12D). Expansion of this cell layer changed slope at Vₘₐₓ≥0.0165 mol·m⁻³·s⁻¹, the point where anoxia first arose. These observations echoed notions from Dionne and others that, among other characteristics, encapsulated insulin-producing cells should ideally have low oxygen consumption rates (Dione et al. In: Lanza R P, Chick W L, editors. Pancreatic Islet Transplantation: Vol III Immunoisolation of Pancreatic Islets. Austin, TX: R G Landes Co; 1994. p. 119-31; Wright and Pohajdak, Cell Transplant. 2001; 10(2): 125-43).

For the effective diffusion coefficients of oxygen in cells (αD)_{cells}, Avgoustiniatos and Colton (Avgoustiniatos and Colton, Ann N Y Acad Sci. 1997; 831(1):145-67) assumed its value to be approximately equal to one third of the product between the diffusion coefficient of oxygen in water at 37° C. (D_{water}=2.78·10⁻⁵ cm²·s⁻¹) and the oxygen solubility coefficient (α_{water}=1.27·10⁻⁹ mol·cm⁻³·mmHg⁻¹). Conversely, Buchwald (Buchwald, Theor Biol Med Model. 2009 Apr. 16; 6(1):5) used D_{water}=3.0·10⁻⁵ cm²·s⁻¹ and α_{water}=1.45·10⁻⁹ mol·cm⁻³·mmHg⁻¹, and assumed the diffusion coefficient of oxygen in cells was D_{cells}=2.0·10⁻⁵ cm²·s⁻¹. Therefore, the analyzed range for (αD)_{cells} was 0.35-4.5·10⁻¹⁴ mol·cm⁻¹·mmHg⁻¹·s⁻¹, where the lower bound was one tenth of the value used by Avgoustiniatos and Colton (Avgoustiniatos and Colton, Ann N Y Acad Sci. 1997; 831(1):145-67) while the upper bound was the product between D_{water} and α_{water} used by Buchwald (Buchwald, Theor Biol Med Model. 2009 Apr. 16; 6(1):5). Increasing the value of (αD)_{cells} led to a concomitant rise in the minimum oxygen partial pressure encountered in the fiber cross-sections. Decreasing (αD)_{cells} to 10% of the base case value resulted in the formation of a 70-μm cell layer with submaximal insulin secretion rate (FIG. 12E). This moderate impact was attributed to cell loading being only 9% of the fiber area.

The effective diffusion coefficient of oxygen in alginate hydrogels used for the shell (αD)₁ and for the cell region (αD)_{c,2} were calculated using Maxwell's relationship for composite media (equation Error! Reference source not found.) as suggested by Avgoustiniatos and Colton (Avgoustiniatos and Colton, Ann N Y Acad Sci. 1997; 831(1):145-67). Conversely, Dulong and Legallais used the Mackie-Meares equation to estimate the effects of changes in alginate concentration on the diffusion coefficient of oxygen in alginate hydrogels (Dulong and Legallais, J Biomech Eng. 2005 Dec. 1; 127(7):1054-61). This report and two from Buchwald (Buchwald all used 2.5·10⁻⁵ cm² s⁻¹ as the value for the diffusion coefficient of oxygen in 1.2% and 3% alginate hydrogels. For the same alginate hydrogels, equation Error! Reference source not found. yielded respectively 2.73 and 2.66 cm²·s⁻¹, when using D_{water}=2.78·10⁻⁵ cm²·s⁻¹ and D_{alginate}=0, i.e., the values reported by Avgoustiniatos and Colton (Avgoustiniatos and Colton, Ann N Y Acad Sci. 1997; 831(1):145-67). However, in the reports from Buchwald as well as those from Dulong and Legallais, the authors did not specify if there were adjustment for the solubility coefficients of oxygen in alginate hydrogels. Therefore, to determine the analyzed range for (αD)₁ and (αD)_{c,2}, we resorted to using the same analyzed range as for (αD)_{cells}. Whereas decreasing the value of (αD)_{cells} to 10% of the base case had a moderate impact, the same decrease in the values of (αD)₁ and (αD)_{c,2} led to formation of an anoxic cell layer spanning 79% of the cell region area, and the absence of a cell layer with maximum insulin secretion rate (FIG. 12F). This difference in the impact of the effective diffusion coefficients was attributed to alginate hydrogel content being higher than that of cells in the fiber cross-sections.

The analyzed core-annulus-shell fiber aimed for all of the cells to be producing insulin at the maximum rate by targeting a minimum oxygen concentration of 5.1 mmHg. However, in the sensitivity analysis, fiber cross-sections with anoxic cell layers were observed when increasing Vₘₐₓ, as well as when decreasing P_{E}, (αD)₁ and (αD)_{c,2}. Future theoretical studies are proposed to look at the interactions between these variables by factorial sensitivity analyses. In a preliminary analysis, we found that a simultaneous ±25% change in the values of Vₘₐₓ, (αD)₁ and (αD)_{c,2} might be a pragmatic safety margin for choosing the cell volume fraction during fiber design. Applying this safety margin was equivalent to designing a fiber for a 10-mmHg decrease in P_{E}. However, besides model parameters, there are other factors that might influence encapsulation device performance in vivo, such as variations in cell loading along the fiber and/or deviations from target fiber layer dimensions. Thus, a second sensitivity analysis was performed to account for such variability and assess a range of different fiber designs.

### Sensitivity Analysis of Cell Loading and Fiber Dimensions

This sensitivity analysis focused on the main fiber design variables that can be controlled during fabrication. First, we explored the effects of variability in the cell loading, thereby the cell volume fraction E in the cell region. The analyzed range for E was 0-100%; whereas the feasibility of bioprinting at such high cell concentration remains to be tested, the models reported by Iwata (Iwata et al., Artif Organs. 2018; 42(8):E168-85) and Avgoustiniatos (Avgoustiniatos and Colton, Ann N Y Acad Sci. 1997; 831(1):145-67) both explored similar scenarios. Decreasing E below the base case value led to a rise in the minimum oxygen partial pressure encountered in the fiber cross-sections. As shown in FIG. 13A, increasing ε resulted in the formation and rapid thickening of the cell layer with submaximal insulin secretion rate. Expansion of this cell layer was reduced with anoxic layer formation at ε≥25.3%. These observations were attributed to E affecting both the oxygen consumption rate of the cell region q_{O}_{₂} and effective diffusion coefficient of oxygen in the cell region (αD)₂ (equations 1 & 2). Therefore, an increase in E led to higher and lower values of q_{O}_{₂} and (αD)₂, respectively. Next, shell thickness was varied while maintaining constant acellular core diameter and annulus cell region thickness. The analyzed range was 15-350 μm, where the lower bound was one tenth of shell thickness in the base case and the upper bound was equivalent to the immunoprotective membrane thickness in islet microencapsulation, i.e., a 150-μm islet in a 500-μm microcapsule. Hence, fiber diameter was #1 mm except for the base case. Decreasing shell thickness below the base case value led to a rise in the minimum oxygen partial pressure encountered in the fiber cross-sections. As shown in FIG. 13B, increasing shell thickness resulted in the formation and gradual thickening of the cell layer with submaximal insulin secretion rate. The rate of expansion of this cell layer decreased with anoxic layer formation at shell thickness≥207 μm. At the upper bound of the range, cells with maximum insulin secretion rate accounted for only 35.3% of the cell region, while anoxic cells accounted for 15.5% of the cell region area.

The effects of variability in acellular core radius were initially explored while maintaining constant annulus cell area and shell thickness. Hence, annulus cell region thickness was ≠200 μm except for the base case. The analyzed range was the same as for shell thickness, 15-350 μm. Acellular core radii above the base case value led to a rise in the minimum oxygen partial pressure encountered in the fiber cross-sections. As shown in FIG. 13C, decreases in acellular core radius resulted in a gradual increase in annulus thickness and the formation and gradual thickening of the cell layer with submaximal insulin secretion rate. The rate of expansion of this cell layer decreased with anoxic layer formation at acellular core radius≤97 μm. At the lower bound of the range, cells with maximum insulin secretion rate accounted for only 66.6% of the cell region, while anoxic cells accounted for 5.6% of the cell region area.

The above was followed by varying acellular core radius while maintaining constant annulus cell thickness. Hence, annulus cell region area was ≠3.14·10⁻² mm² except for the base case. Acellular core radii below the base case value led to a rise in the minimum oxygen partial pressure encountered in the fiber cross-sections. Increases in acellular core radius resulted in in a gradual increase in annulus thickness and, as shown in FIG. 13D, the formation and gradual thickening of the cell layer with submaximal insulin secretion rate, and anoxic cell layer formation at acellular core radius≤310 μm. At the upper bound of the range, cells with maximum insulin secretion rate accounted for only 55% of the cell region, while anoxic cells accounted for 1.3% of the cell region area. While at the lower bound of the range, all cells had maximum insulin secretion rate but due to the decrease in annulus area, the cell loading was reduced from 9% to 8.1%.

The differences in the two variations of acellular core radius occurred because maintaining constant annulus thickness altered the cell region area of the resulting fiber cross sections. In turn, given the constant cell volume fraction E, these alterations affected the cell loading. Conversely, increasing acellular core radius while maintaining constant cell region area did not cause an increase in cell loading.

Fiber cross-sections with anoxic cell layers were observed when increasing E, shell thickness and acellular core radius with constant annulus thickness, as well as for decreasing acellular core radius with constant annulus area. Variability in fiber dimensions and E during printing might be much smaller than ranges studied here, i.e., deviations of a few dozen microns from desired fiber dimensions or cell distribution. However, the perturbations leading to variability in fiber parameters might not to be perfectly orthogonal and cause interactions. For example, a slight variation in shell thickness associated with a constriction of the annulus could also lead to a change in the dimensions of the acellular core. These variations are still being characterized and their interactions should be explored in the near future by factorial sensitivity analyses. Moreover, sophisticated finite element modelling approaches (Dulong and Legallais, Biotechnol Bioeng. 2007 Apr. 1; 96(5):990-8; Johnson et al., Chem EngSci. 2009; 64(22):4470-87) could be used to study the effect of fiber design on oxygenation and secretory capacity of individual cell aggregates, something that cannot be calculated using our theoretical model.

Our observations were used to derive general concepts for fiber design: (i) increases in shell thickness and annulus cell region area (resulting from increases in acellular core radius or annulus thickening) raise the likelihood of oxygen limitations; (ii) decreases in annulus cell region thickness accompanied by increases in acellular core radius reduce the likelihood of oxygen limitations. In vitro experiments are ongoing where cells encapsulated in core-annulus-shell fibers are exposed to controlled oxygen concentrations and oxygen limitations at the cellular level are detected using fluorescent probes.

### Conclusions

Passive diffusion to encapsulated insulin-producing cells remains an important option to develop, since this could provide essentially carefree reversal of diabetes, circumventing the need for daily oxygen refills as well as the risks inherent to using a more complicated device over a lifetime. Bioprinted alginate fibers are potentially retrievable like macroencapsulation devices, and also enable encapsulation at cell loadings comparable to those in microcapsules (2.7% for 150-μm cell aggregate in 500-μm capsule). Radial displacement of the cell region in the core-annulus-shell fibers allowed for higher encapsulation cell loadings than core-shell fibers. Based on the sensitivity analysis for fiber parameters, we provided strategies to aid in the selection of fiber dimensions during design. Moreover, the model presented here showed aiming for maximum insulin secretion rate throughout the cell region in a core-annulus-shell fiber did not compromise its secretory capacity. It is herein recognized that this strategy may be used to avoid oxygen limitations and the subsequent release of immunogenic factors such as DAMPs that elicit graft failure (Paredes-Juarez et al., Sci Rep. 2015; 5(September):1-12).

### Example 2: In Silico Analysis of Annulus Encapsulation Fibers

As shown in this example, modeling can be used to provide reliable estimates for fiber dimensions for experimental test. This Example further demonstrates that the annulus design may reduce total transplant fiber length by about ½ versus solid core, single shell architecture fiber types. This Example additionally demonstrates maximal cell loading based on O₂ utilization down to ˜1 mmHg and correlation with insulin secretion.

Oxygen transport was selected to model at least because 1) oxygen is less soluble than, for example, glucose, but is consumed at a similar rate, 2) the model encapsulates, for example, islet oxygenation by diffusion, and 3) insulin secretion, for example, is oxygen-dependent.

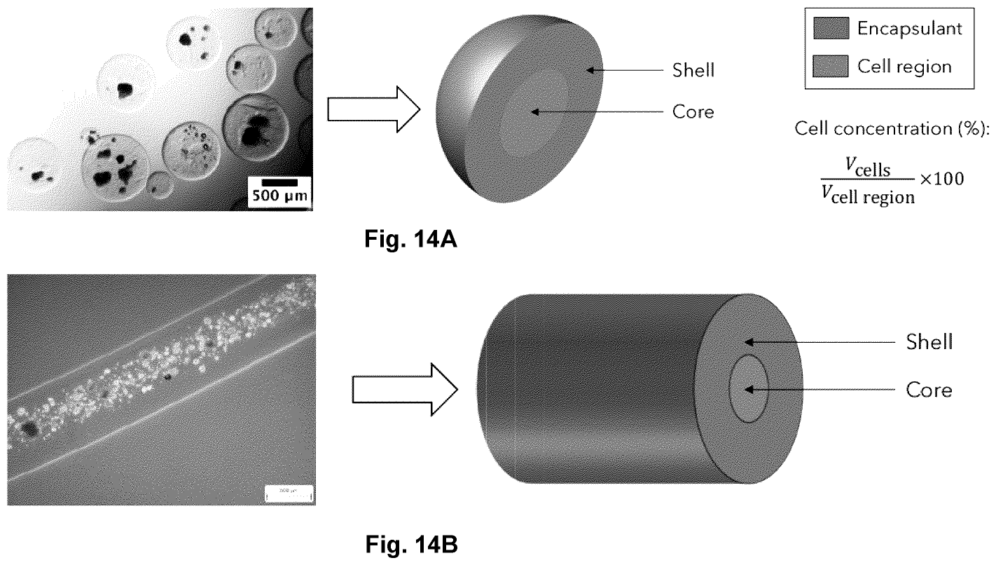

FIGS. 14A-14B illustrate simplified geometries used for the analytical model. An actual image of spherical geometry is shown (FIG. 14A, left) along with an illustrative depiction of the spherical geometry (FIG. 14A, right). Also shown is an actual image of cylindrical geometry (FIG. 14B, left), along with an illustrative depiction of the cylindrical geometry (FIG. 14B, right). As shown, the shell in each example comprises encapsulant, while the core comprises the cell region. Cell concentration within the cell region is defined as volume of cells divided by the volume of the cell region, multiplied by 100.

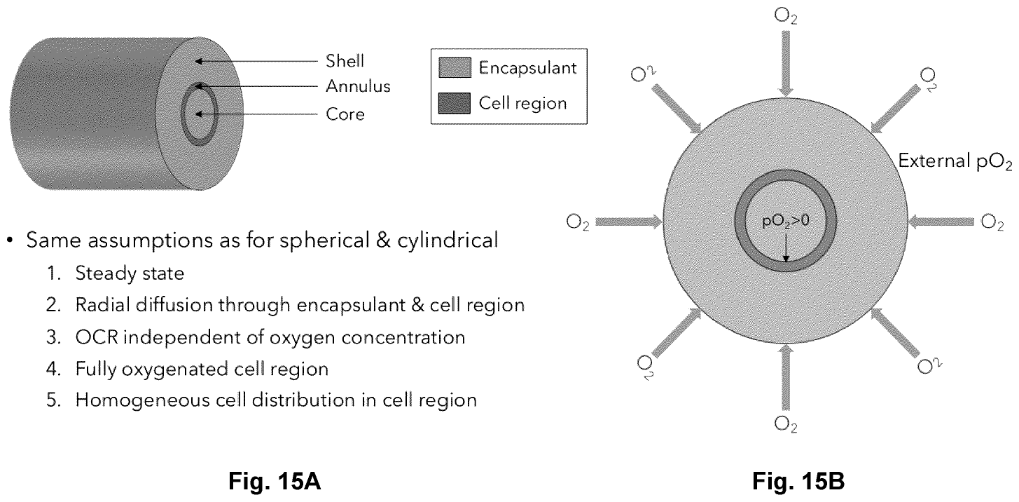

FIGS. 15A-15B illustrate simplified geometries for annulus fibers of the present disclosure. FIG. 15A illustratively shows the core, annulus, and shell regions. The cell region is contained within the annulus region, and the shell and core regions comprise the encapsulant. FIG. 15B depicts a cross-sectional view of an annulus fiber, illustratively showing external partial pressure of O₂ (external pO₂), and that in the modeling, pO₂ is assumed to be greater than 0 in the core region.

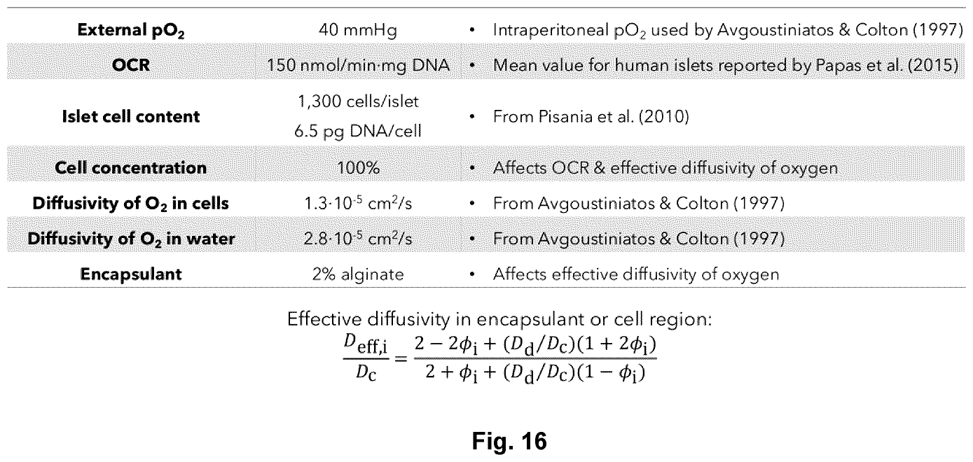

Same assumptions were used for modeling oxygen transport for the annulus fiber as assumptions used for the spherical and cylindrical fiber types. Specifically, it was assumed that 1) the system has reached steady-state conditions, 2) that there is radial diffusion through the encapsulant and cell regions, 3) that oxygen consumption rate (OCR) is independent of oxygen concentration, 4) that the cell region is fully oxygenated, and 5) that cell distribution is homogeneous within the cell region. Other assumptions on model parameters are illustrated in the table shown at FIG. 16.

### Oxygen Profiler

Model inputs used were various spherical, cylindrical, and annulus device dimensions. The modeling process comprised mass transfer calculation in 1 μm steps. The output of the modeling procedure was partial pressure as a function of radial distance (r_{T}) from a center of the core to the outer boundary of the particular device.

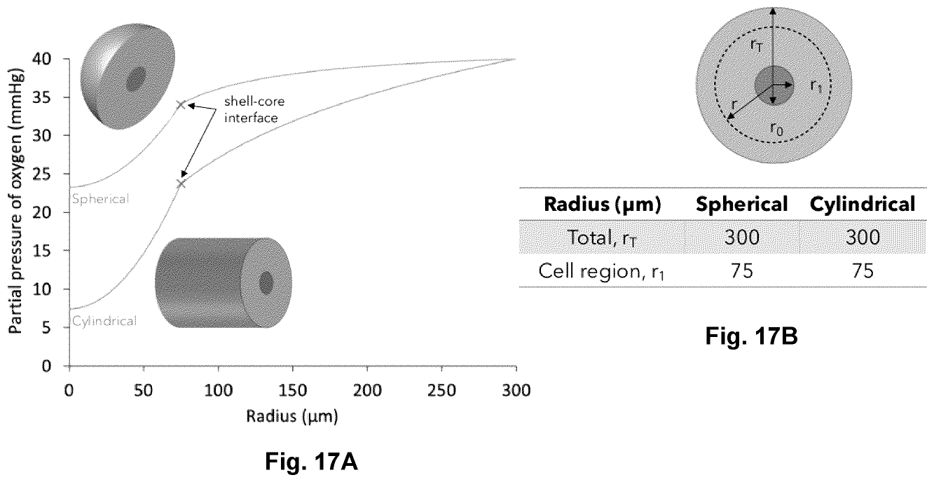

Model output for spherical and cylindrical geometries is shown at FIG. 17A. For each partial pressure trace, the shell-core interface location in terms of radius is depicted. Device dimensions used in the generation of the data of FIG. 17A are depicted at FIG. 17B.

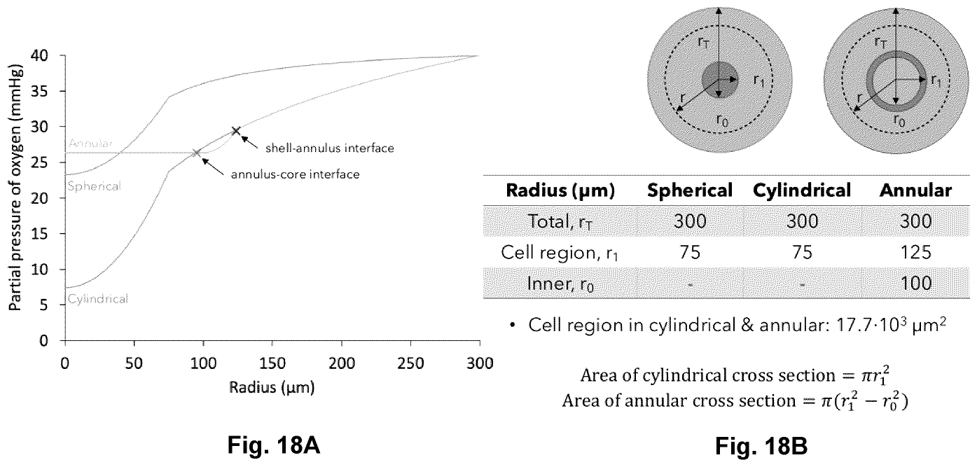

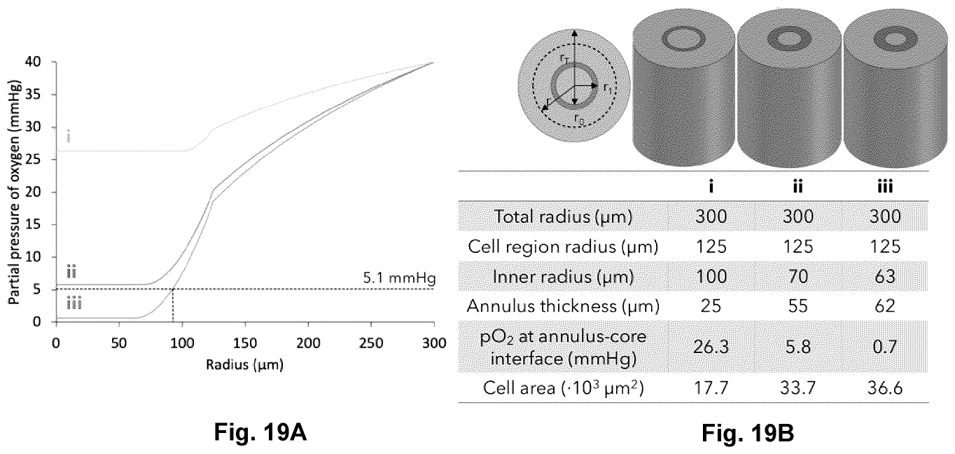

FIG. 18A illustrates an oxygen profile for an annulus device, overlaid with the oxygen profiles of the spherical and cylindrical devices. Partial pressure at the annulus-core interface was 26.3 mmHg. Shown at FIG. 18A is the location in terms of radius of the annulus-core interface and the shell-annulus interface. Device dimensions used in the generation of the data of FIG. 18A are depicted at FIG. 18B. In this example, cell region in the both the cylindrical and annulus fibers was 17.703 m². Oxygen profiles for other annulus regions are depicted at FIG. 19A, along with corresponding device dimensions and other various parameters (FIG. 19B).

The results of the oxygen profiling studies indicate a decrease in pO₂ at the annulus-core interface as annulus thickness is increased.

### Insulin Secretion Profiler

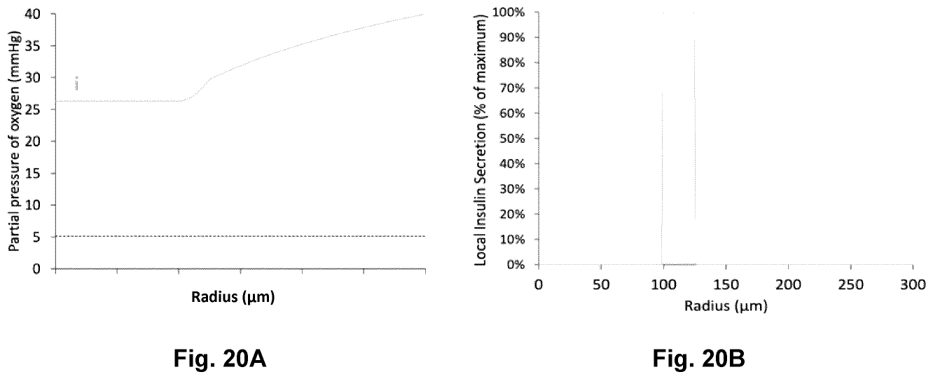

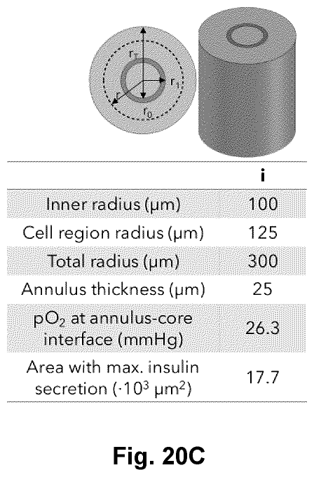

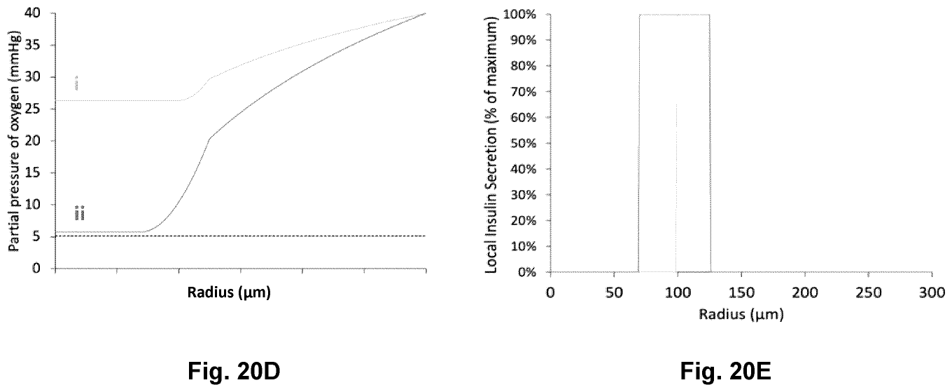

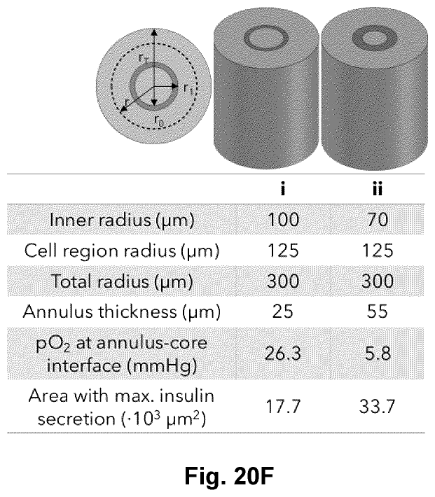

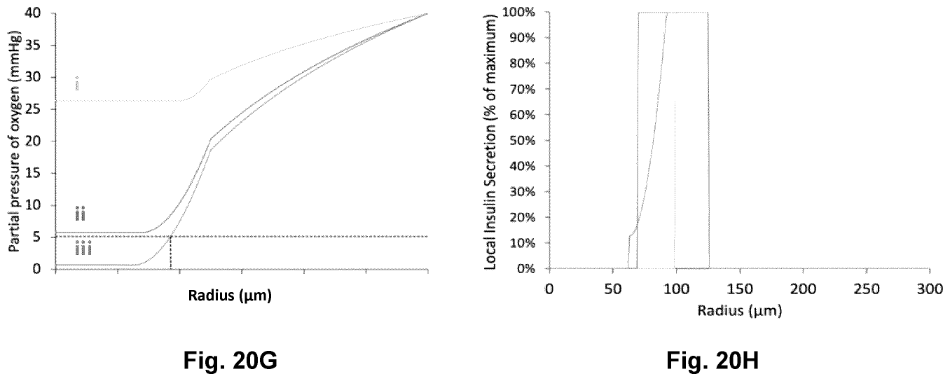

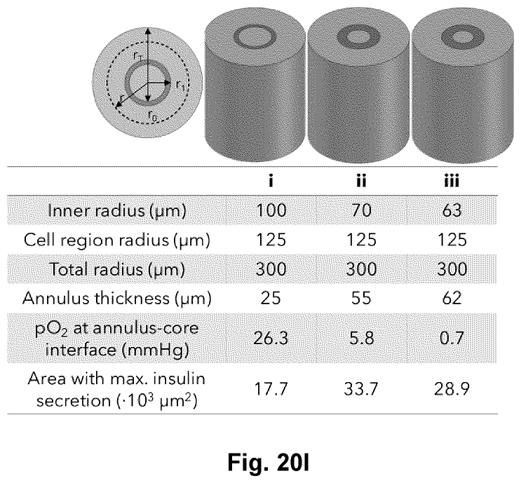

Model inputs used were the device dimensions that were used in the oxygen profiler modeling procedure, along with the oxygen profiles obtained from the oxygen profiler modeling procedure. The modeling process included conversion of the inputs to local insulin secretion (as % of normal). The output of the modeling procedure was insulin secretion as a function of radial distance, and cell region area with normal insulin secretion. Shown at FIG. 20A is partial pressure of oxygen as a function of radial distance for a first annulus device, and local insulin secretion (% of maximum) as a function of radial distance for the first device is depicted at FIG. 20B. First device dimensions and relevant parameters are shown at FIG. 20C.

Shown at FIG. 20D is partial pressure of oxygen as a function of radial distance for a second annulus device, and local insulin secretion (% of maximum) as a function of radial distance for the second device is depicted at FIG. 20E. Second device dimensions and relevant parameters are shown at FIG. 16F. For reference, the data corresponding to the first device (refer to FIGS. 20A-20C) are also shown in FIGS. 20D-20F.

Shown at FIG. 20G is partial pressure of oxygen as a function of radial distance for a third annulus device, and local insulin secretion (% of maximum) as a function of radial distance for the third device is depicted at FIG. 20H. Third device dimensions and relevant parameters are shown at FIG. 20I. For reference, the data corresponding to the first device (refer to FIGS. 20A-20C), and the second device (refer to FIGS. 20D-20F) are also shown in FIGS. 20G-20I.

The data of FIGS. 20A-20I illustrate that the area with maximal insulin secretion is a function of annulus thickness, and that the maximal insulin secretion increased from the first device to the second device (annulus thickness increase from 25 μm to 55 μm), but that the area with maximal insulin secretion decreased from the second device to the third device even though annulus thickness increased from 55 μm to 62 μm.

### Annulus Configuration Finder

In this procedure, model inputs used comprised maximal total radius, and optionally included annulus or shell thickness. The modeling process included searching for maximum cell region area and minimum difference to target pO₂ at the annulus-core interface. Output of the modeling process included dimensions for annulus configuration with maximum cell region or insulin-secreting area.

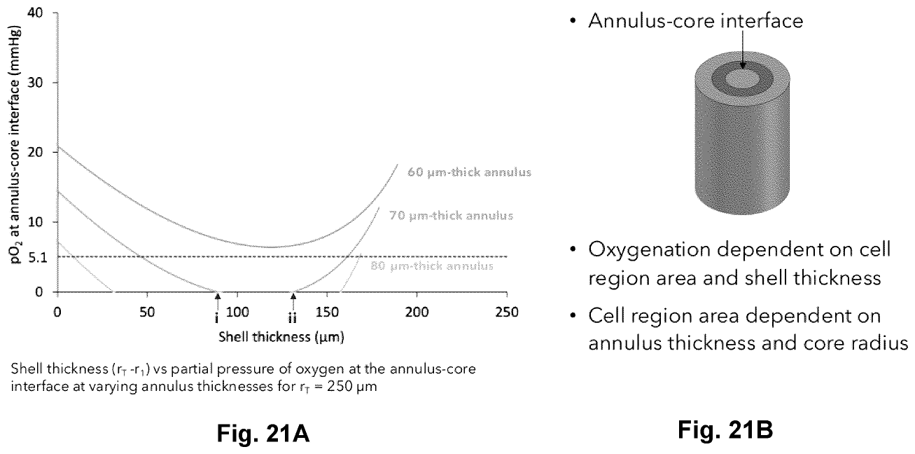

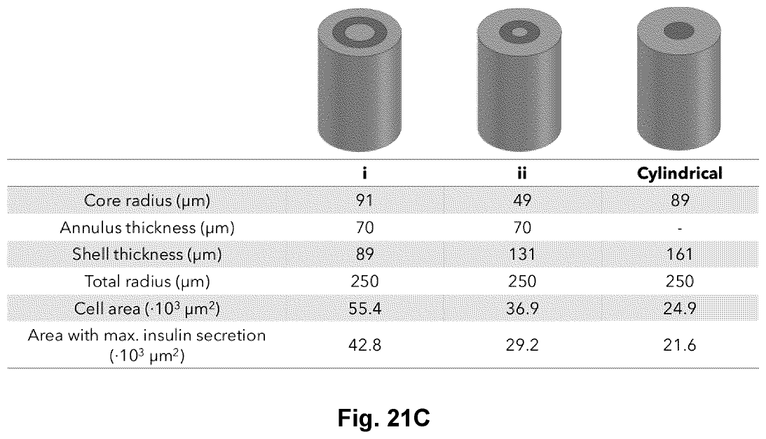

FIG. 21A illustrates pO₂ at the annulus-core interface (refer to FIG. 21B) of annulus device of varying annulus thickness (60 μm, 70 μm, 80 μm), as a function of shell thickness (total radius (r_{T}) minus cell radius (r₁). The dashed line at FIG. 21A represents 5.1 mmHg. Shown are two points (illustrated “i” and “ii”), which were further analyzed. Turning to FIG. 21C, dimensions and various output parameters for each of the devices defined by the points “i” and “ii” are shown along with dimensions and various output parameters of a particular cylindrical device. The data indicates that it may be possible to increase the area with maximal insulin secretion about two-fold or more via employment of an annulus device as compared with a cylindrical device, and that the area with maximum insulin secretion in annulus devices is a function of the relationship between core radius, annulus thickness, and shell thickness.

### Example 3: Theoretical Oxygen Transport and Insulin Secretion Rate, and FITC-Dextran Release Studies for Solid Core, Single Shell Architectures

Encapsulation is a key final manufacturing step for beta cell replacement therapy (among other cell replacement therapies). Devices such as alginate beads, fibers and slabs aim to protect encapsulated cells from immune rejection while allowing, for example, the transport of insulin, glucose and oxygen. Compared to conventional spherical alginate beads, an important advantage of bioprinted fiber immunoprotection is that the encapsulated cells can be restricted to a core that is isolated from the device surface by a semipermeable outer shell. The encapsulated cells are protected by this shell and oxygenated via passive diffusion such that it is primarily the oxygen transport that limits the volume of cells that can be encapsulated.

The objectives of this Example included 1) modeling of oxygen transport to design core-shell fiber configurations with a fully oxygenated core region, 2) optimization of the application to the treatment of diabetes using a bilinear model to simulate impact of the local oxygen partial pressures on the insulin secretion rate, and 3) find minimum shell thicknesses that are sufficiently immunoprotective (i.e., exclude IgG) using FITC-dextran conjugates with defined molecular ranges.

In similar fashion to that discussed above with regard to Example 1, calculations for oxygenation of fibers implanted intraperitoneally were based on published analytical models (Iwata et al., Artif Organs. 2018; 42(8):E168-E185; Avgoustiniatos et al., Ann N Y Acad Sci. 1997; 831(1):145-167), and were carried out using MS Excel and Visual Basic scripts. Values for islet oxygen consumption rate (q_{O2}), effective diffusivities of oxygen in islets (Di) and alginate hydrogel (D_{H}), and intraperitoneal oxygen partial pressure were all retrieved from the literature (Papas et al., PLoS One. 2015; 10(8):e0134428; Safley et al., Transplantation. 2020; 104(2):259-269). Estimates of insulin secretion rate were based on a model (Johnson et al., Chem Eng Sci. 2009; 64(22):4470-4487) where beta cells are assumed to retain their maximal insulin secretion rate at local oxygen partial pressures above 5.1 mmHg.

### Analytical Oxygen Transfer Model

An example of an islet-loaded bioprinted fiber is depicted at FIG. 5A. As shown at FIG. 5A, islets were distributed homogeneously in the core of the printed fibers. Fiber geometry of the core was simplified (FIG. 5B-5C) by assuming a constant cell volume fraction (X). Effective diffusivity (D_{C}) and consumption rate (q′_{O2}) of oxygen in this layer were adjusted accordingly:

$${D}_{C} = {D}_{H} \frac{2 - 2 X + ( 1 + 2 X ) {D}_{1} / {D}_{H}}{2 + X + ( 1 - X ) {D}_{1} / {D}_{H}} \qquad ( 17 )$$

$${q}_{{O}_{2}}^{′} = {X}_{{q}_{{O}_{2}}} \qquad ( 18 )$$

To account for core layers deprived of oxygen and/or with submaximal insulin secretion arising, the potential boundaries of these layers in terms of radial distance (r_{A} and r_{I}) were included. Thus, the steady-state diffusion and reaction rate of oxygen through the shell and the core, respectively, were written as:

$$2 π r {D}_{H} \frac{\partial p {O}_{2} ( r )}{\partial r} = {q}_{{O}_{2}}^{′} π ( {R}_{2}^{2} - {r}_{A}^{2} ) \qquad ( 19 )$$

$$2 π {rD}_{1} \frac{\partial p {O}_{2} ( r )}{\partial r} = {q}_{{O}_{2}}^{′} π ( {r}^{2} - {r}_{A}^{2} ) \qquad ( 20 )$$

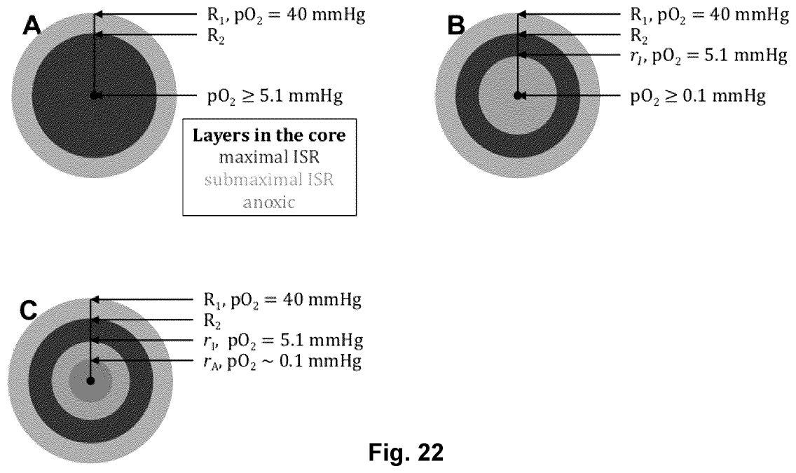

These equations were solved iteratively by assuming 1) constant oxygen concentration surrounding the fiber, 2) radial diffusion only (i.e., axial diffusion negligible), 3) zero-order kinetics for oxygen consumption, and 4) submaximal insulin secretion rate=pO₂ (r)/5.1 using the sets of boundary conditions shown in FIGS. 22A-22C.

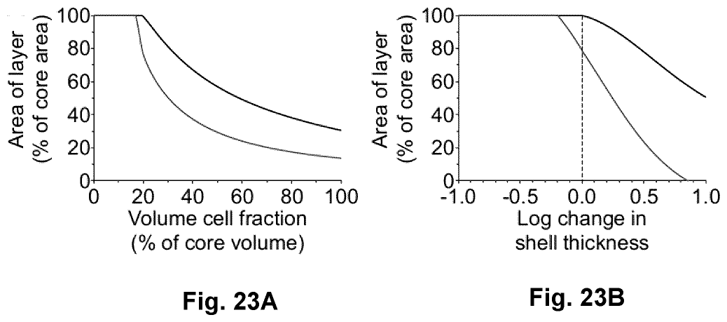

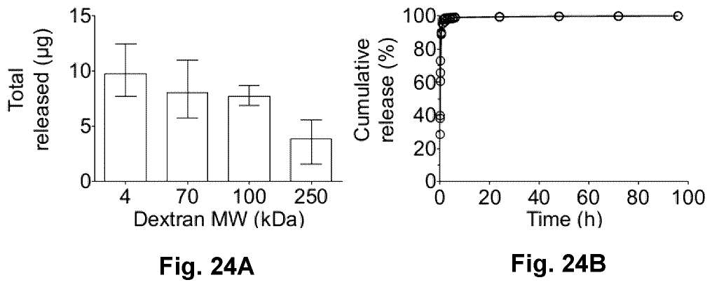

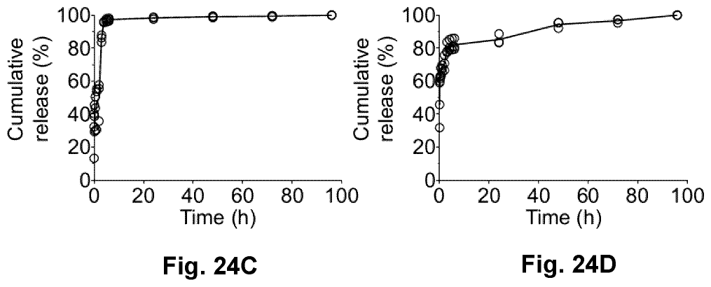

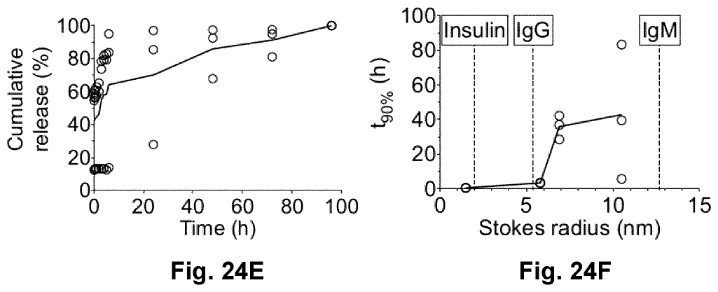

Cell volume fraction loading below 20% resulted in maximal insulin secretion rate throughout the core (FIG. 23A), and fibers with thinner shells improved core oxygenation (FIG. 23B). Minimum shell thicknesses that are sufficiently immunoprotective (i.e., exclude IgG) were examined using FITC-dextran conjugates with defined molecular weight ranges. The data is depicted at FIGS. 24A-24F, and illustrates FITC-dextran recovered from supernatants of four types of fibers (FIGS. 24A-24E), as well as a graph showing time for 90% release of total dextran as a function of molecule size (FIG. 24F). At FIG. 24F, dextran MW were converted to hydrodynamic radii based on published literature (Schnell et al., J Biomed Opt. 2008; 13(6):064037).

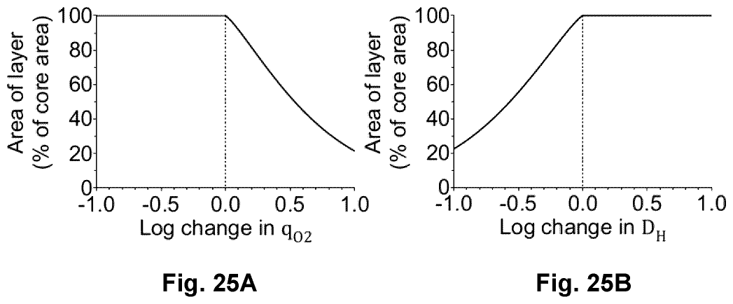

A one-factor at a time sensitivity analysis of modeled parameters was performed (FIGS. 25A-25B). Depicted are effects of uncertainty in values of islet oxygen consumption rate (FIG. 25A) and effective diffusivity of oxygen in alginate hydrogel (FIG. 25B) on oxygenated layer. Similar analyses were performed for the effective oxygen diffusivity of oxygen in islets and local oxygen partial pressure at which beta cells retain maximal insulin secretion rate (data not shown). The former had a negligible effect on oxygen transport due to assumption of constant cell volume fraction. The latter did not have an impact on core oxygenation.

Hence, it was found that loading the maximum number of oxygenated cells could reduce the proportion of cells with maximal insulin secretion rate, and that separation from the oxygen source (i.e., shell thickness) impacts core oxygenation and fiber encapsulation capacity. It was also found that alginate in the semipermeable outer shell may need to be modified in order to exclude IgG at reduced thicknesses. Taken together, this Example demonstrates that oxygen uptake of encapsulated cells, which is a key driver of oxygen transport, requires careful measurement and validation.

### Example 4: In Vitro Studies of C/S Fibers

### Mechanical Strength of Zwitterionic Alginate Fibers

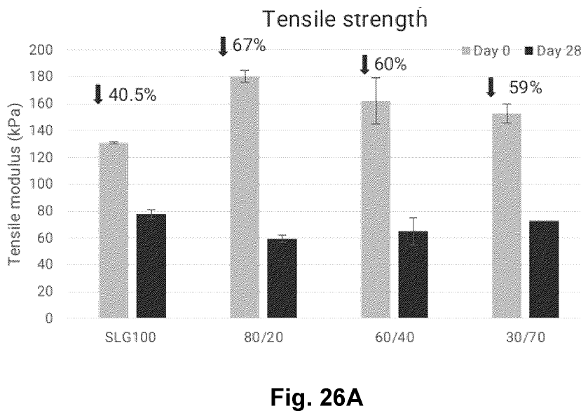

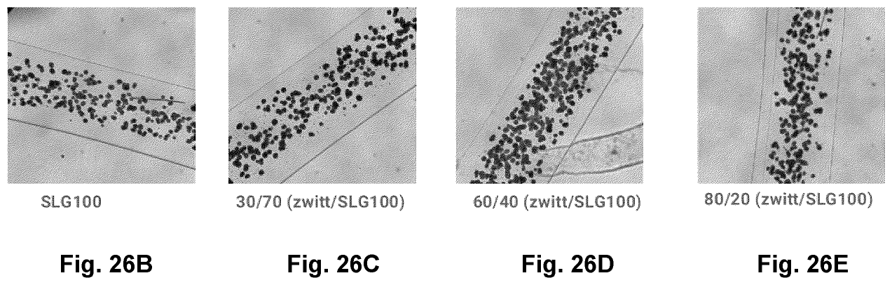

Min6 clusters were printed in C/S fibers with shells composed of different ratios of zwitterionic alginate to SLG100 as shown, cultured for 4 days in media, and then 28 days in Krebs Ringer's buffer (to approximate in vivo conditions). Buffer was changed every 3 days. The higher the zwitterionic alginate percentage in the blend, a) the higher the mechanical strength after print but (b) the higher percentage decrease in mechanical strength one month post print (FIG. 26A). FIGS. 26B-26E are brightfield images of fibers corresponding to the data depicted at FIG. 26A.

PHI Viability Studies on Fibers Printed with Shell Composed of Various Anti-FBR Materials

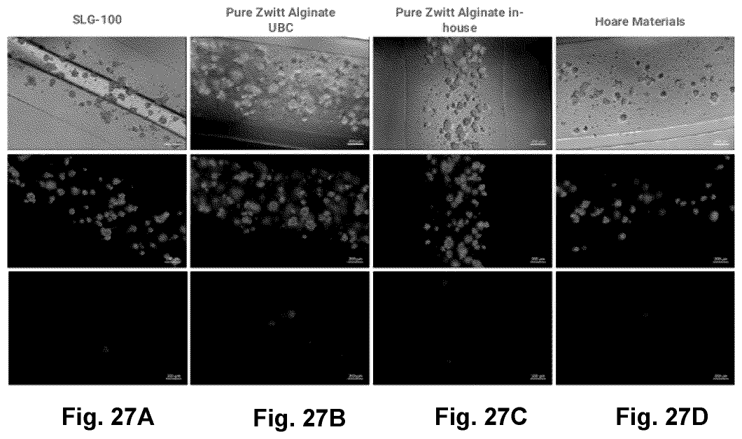

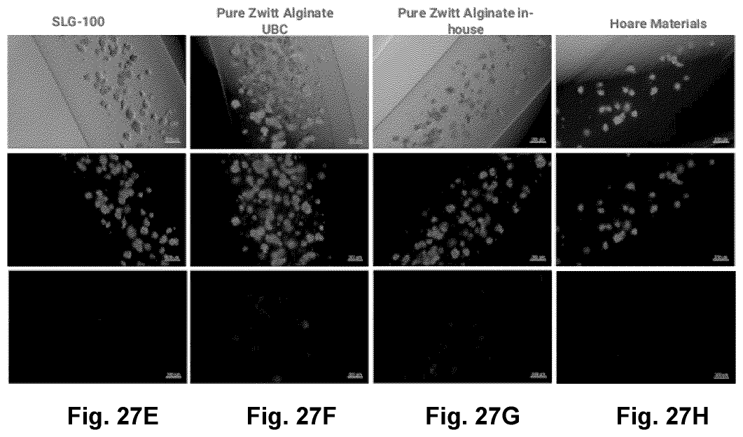

C/S fibers printed with a shell composed of various anti-FBR materials were examined in terms of whether the particular anti-FBR materials supported primary human islet (PHI) viability. For the experiments, the core was always composed of 1.5% SLG100, and the density of cells was 30,000 islet equivalents (IEQ)/mL. Shell materials tested include SLG100 (control), pure zwitterionic alginate (McLachlan group, University of British Columbia), pure zwitterionic alginate (synthesized in house), and DMAPS-Ald or hydrazide DMAPS-Hzd (Hoare Group, refer to Table 1). Representative live/dead images of PHI at 24 hours for the various shell materials tested are shown at FIGS. 27A-27D. Representative live/dead images of PHI at 7 days for the various shell materials tested are shown at FIGS. 27E-27H. All conditions tested showed high viability 7 days post print.

### Assessment of F-F Adhesion (Optimized Formulation)

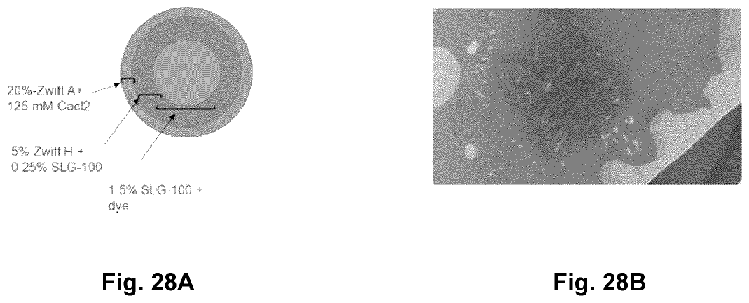

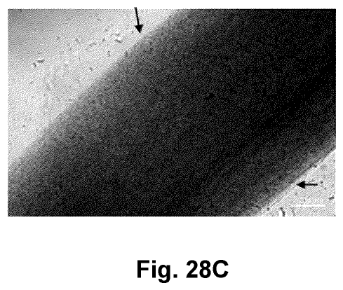

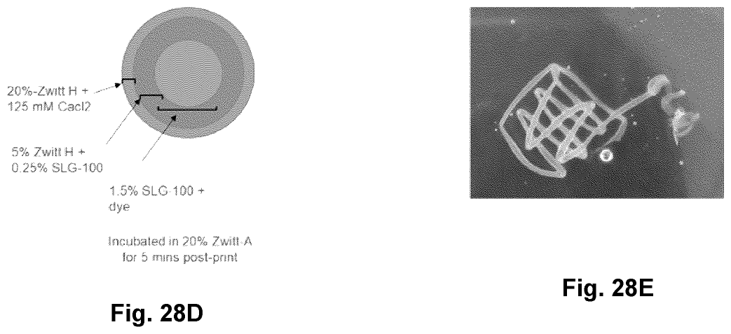

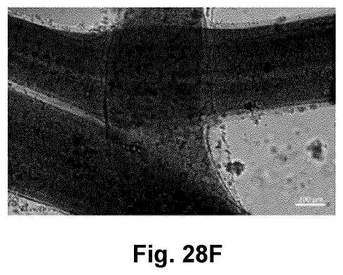

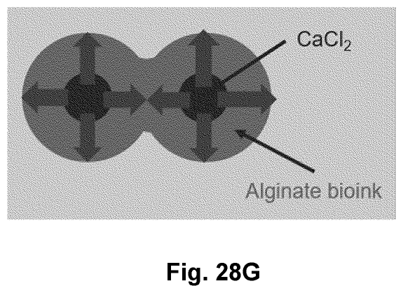

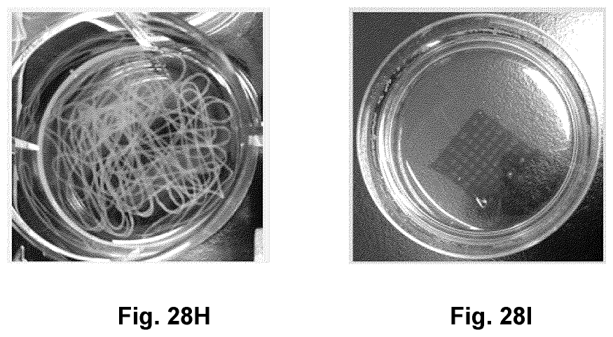

A patterned structure was printed to assess F-F adhesion of annulus fibers using an optimized formulation. Cross-linking of fibers was examined both on chip (FIGS. 28A-28C), and post-print (FIGS. 28D-28F). FIG. 28A depicts a cross-sectional illustration of fibers used in the on-chip cross-linking examination, including indications of components of the core, inner shell, and outer shell. FIG. 28D depicts a cross-sectional illustration of fibers used in the post-print cross-linking examination, including indications of components of the core, inner shell, and outer shell. An image of the patterned structure generated in the on-chip cross-linking experiments is depicted at FIG. 28B, and FIG. 28C shows a high-resolution image of a fiber of the pattern shown in FIG. 28B. An image of the patterned structure generated in the post-print cross-linking experiments is depicted at FIG. 28E, and FIG. 28F shows a high-resolution image of fibers of the pattern shown in FIG. 28E. As can be seen in the high-resolution images, print fidelity appears good for the fibers cross-linked on-chip, yet the fiber unraveled immediately post print. In contrast, print fidelity appeared rather poor for the post-print cross-linking experiments, yet F-F adhesion was maintained.

FIG. 28G depicts an exemplary illustration of inside-out (I-O) cross-linking as disclosed herein. As shown, calcium in the core diffuses outward (refer to block arrows) to cross link one or more external shell layers. Comparative experiments were conducted to assess F-F adhesion for outside-in (O-I) cross linked fibers as compared to I-O cross linked fibers. As shown at FIG. 28H, O-I cross linked fibers exhibited degraded F-F adhesion as compared to I-O cross linked fibers (FIG. 28I). The images of O-I cross linked fibers and I-O cross linked fibers (FIGS. 28H and 28I, respectively), were taken while the corresponding fibers were in phosphate-buffered saline (PBS). As shown, I-O crosslinking may improve fidelity and tissue integrity.

### Example 5: Immune-Protection at Fiber Level in Small Animal Model Using Cell-Based Approach (MSC) and/or Low FBR, Immune-Protective Outer Shell Biomaterial

### Implantation of PHI Fibers in Healthy Immune-Competent Mice

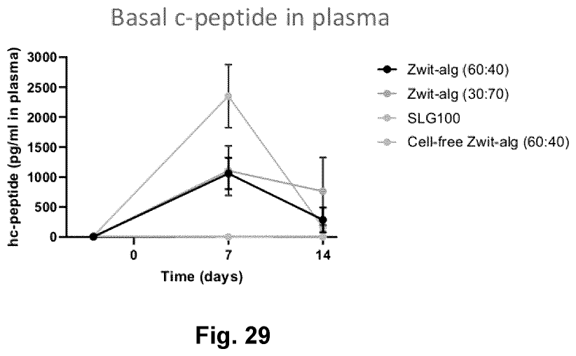

Solid core, single shell fibers (C/S) comprised of varying ratios of zwitterionic alginate and including primary human islet (PHI) cells were implanted into healthy B6 mice. Plasma C-peptide was measured on days −3, 7, and 14 (FIG. 29) after modified oral glucose tolerance test (OGTT), and blood sugar was measured using a glucometer. The data is suggestive that zwitterionic alginate may reduce FBR and improve fiber functionality.

### Implantation of PHI Fibers to Diabetic Immune-Competent Mice

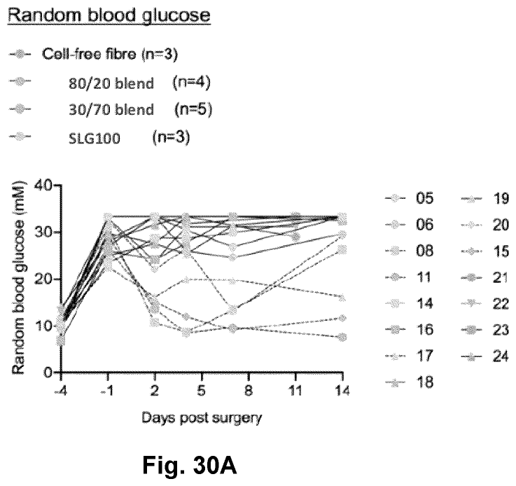

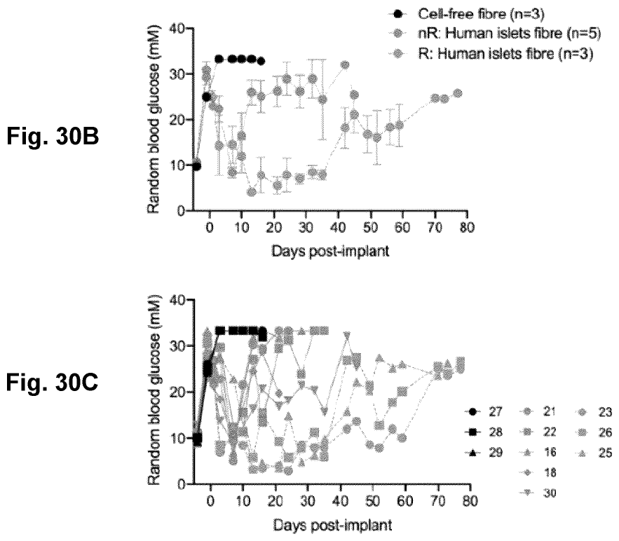

C/S fibers comprised of varying ratios of zwitterionic alginate and including PHI cells were implanted into diabetic immune-competent mice, and blood glucose was measured. FIG. 30A illustrates blood glucose measured as a function of days post surgery. The data is indicative that zwitterionic modification may help reduce FBR.

### Human Islet Fiber Efficacy in Reversal of Diabetes Mellitus in STZ-Induced Diabetic B6 Mice

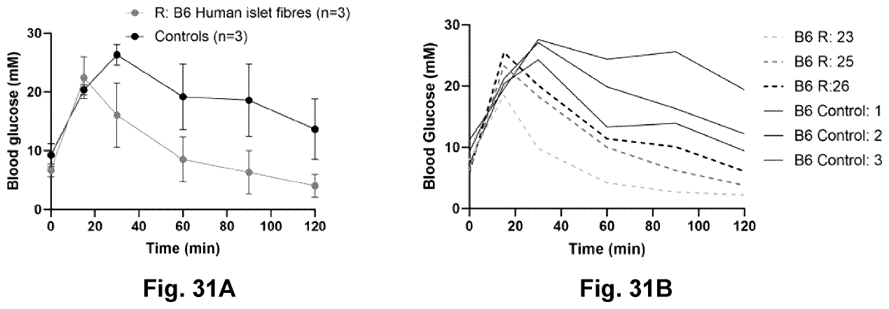

Efficacy of bioprinted C/S human islet fibers (unmodified alginate in the core and outer shell) in reinstating euglycemia in immune-competent diabetic C57BL/6 mice was examined. Diabetes was induced by a single injection of a high dose STZ (200 mg) intraperitoneally. Diabetes was confirmed by 2× blood glucose levels>30 mmol/L. FIGS. 31A-31B illustrate blood glucose measurements after intraperitoneal glucose tolerance test (IPGTT) in long-term responders (day 32-36 post-implantation). Raw data is shown at FIG. 31B, and averaged data generated from the data of FIG. 31B is depicted at FIG. 31A. For the experiments, each mouse was fasted for 6 hours and administered 2 g/kg of glucose via IP injection (day 32-36 post implantation). Controls were the same age non-diabetic mice. The data indicates that the bioprinted human islet tissue implants were effective at controlling blood glucose levels following the glucose bolus.

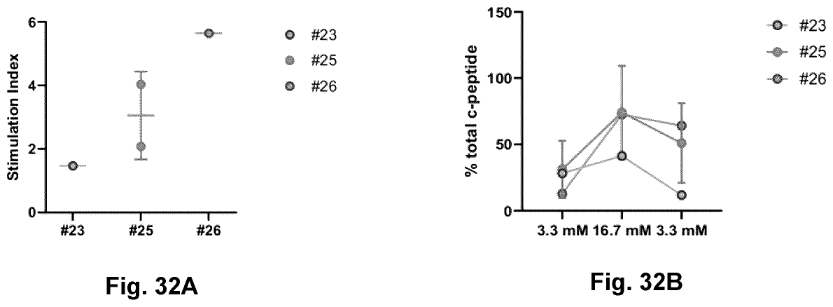

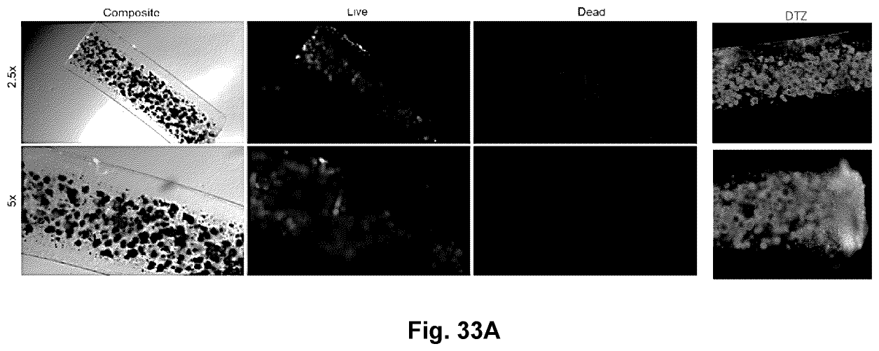

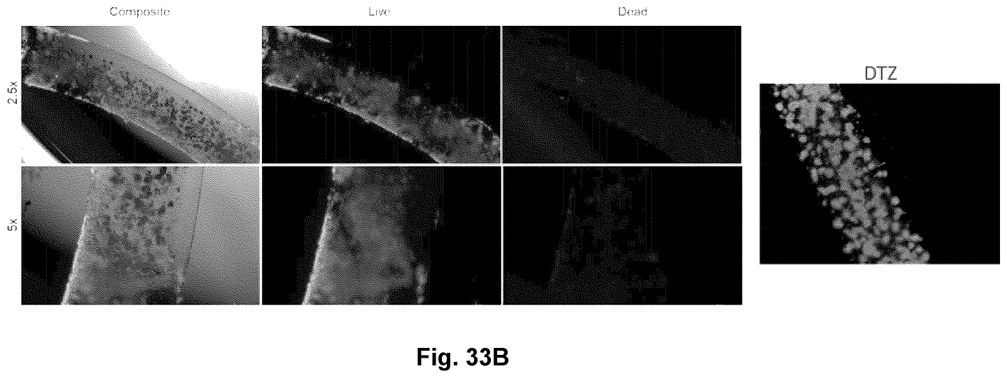

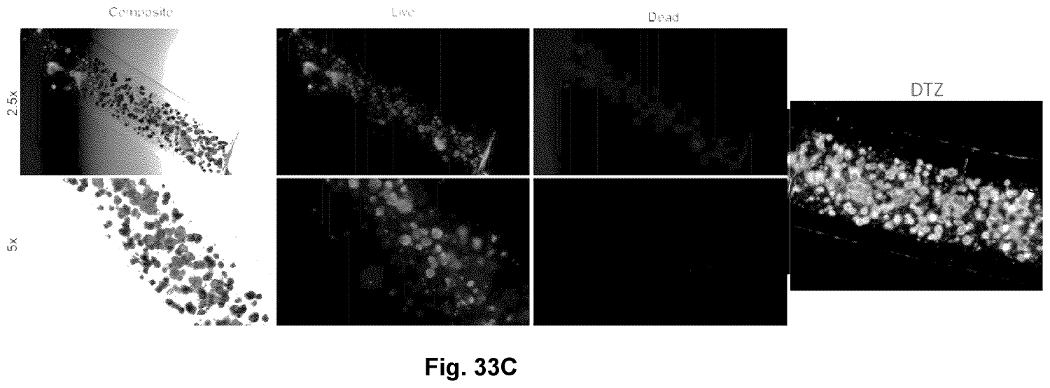

Glucose-stimulated insulin secretion (GSIS) tests were performed on retrieved islet-containing fibers ex vivo. Fibers were retrieved at about 80 days post-implantation. Data from three representative mice (i.e., fibers retrieved from said mice) is depicted at FIGS. 32A-32B. FIG. 32A illustrates stimulation index for each representative mouse, and FIG. 32B illustrates % total c-peptide vs. concentration. FIGS. 33A-33C depict live/dead staining for fiber devices retrieved from the representative mice discussed with regard to FIGS. 32A-32B. Staining for mouse #23 is depicted at FIG. 33A, staining for mouse #25 is depicted at FIG. 33B, and staining for mouse #26 is depicted at FIG. 33C. GSIS post-explantation demonstrated the tissue from mouse #26 has the highest function. The images show there is more of a fibrotic capsule around the tissue from mouse #25 in particular. Accordingly, the data demonstrates some correlation between FBR and loss of function.

### Human Islet Fiber Efficacy in Reversal of Diabetes Mellitus in STZ-Induced Diabetic NSG Mice

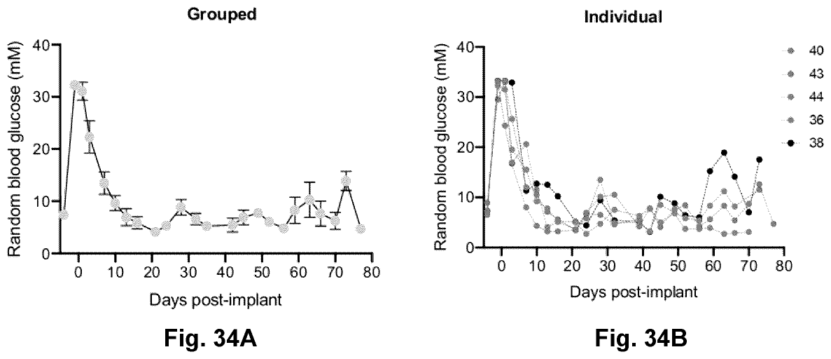

Efficacy of bioprinted C/S human islet fibers (unmodified alginate in core and shell) in reinstating euglycemia in immune-deficient diabetic mice was examined. Diabetes was induced by either a single injection of a single high dose, or multiple low doses of STZ, intraperitoneally. Diabetes was confirmed by 2× blood glucose levels>30 mmol/L. FIG. 34B illustrates random glucose values for individual mice as a function of days post-implant for individual mice, and FIG. 34A shows averaged data from the individual mice of FIG. 34B. This data shows maintenance of blood glucose at normoglycemia in almost all animals for up to 80 days (3000 human IEQ).

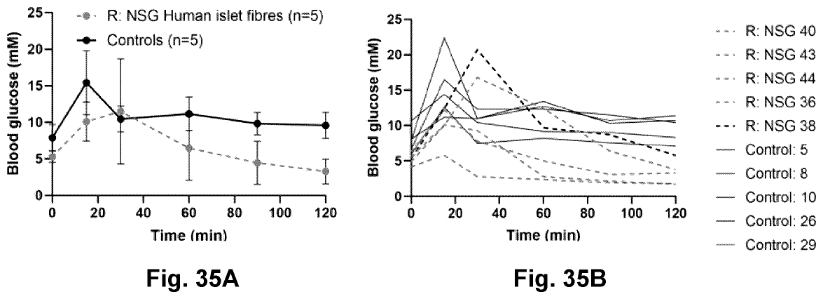

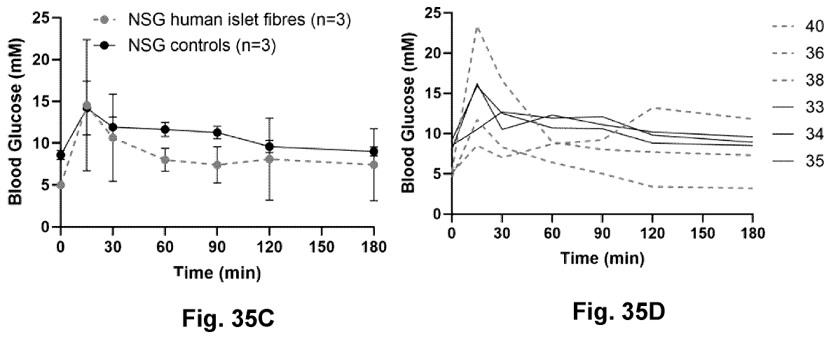

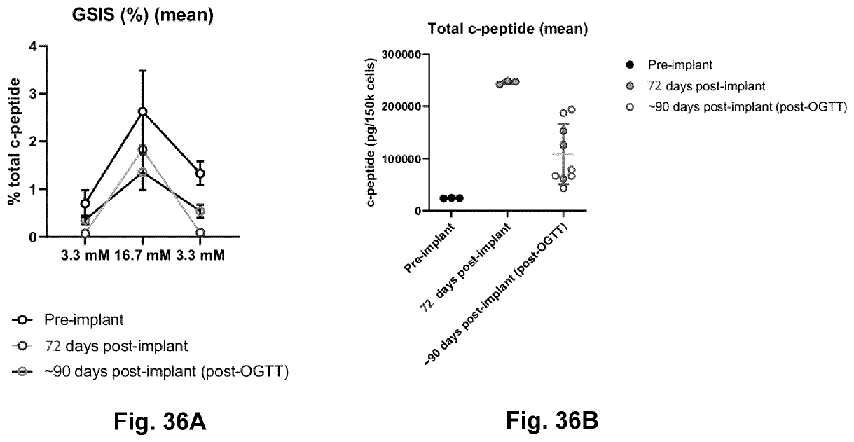

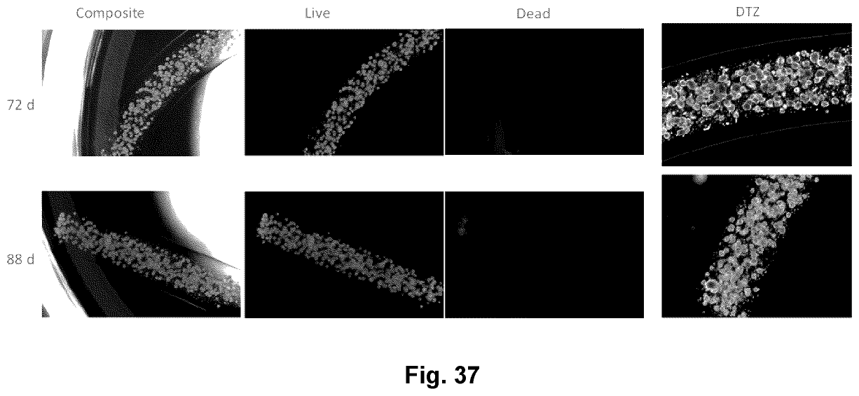

Glucose tolerance tests were conducted at days 36 and 39 post-implant (FIGS. 35A-35B), and at days 82 and 85 days (FIGS. 35C-35D) post implant. For the testing, each mouse was fasted for 4 hours and administered 2 g/kg of glucose via oral gavage. In the depicted experiments, controls were the same aged mice (lacking the C/S device). Data from individual mice are shown at FIGS. 35B and 35D, and averaged data is depicted at FIGS. 35A and 35C. Plasma was also collected for GSIS tests. Representative data is depicted at FIGS. 36A-36B. FIG. 36A depicts % total c-peptide as a function of concentration for pre-implant, 72 days post-implant, and 90 days post-implant (post-OGTT). FIG. 36B depicts c-peptide concentration (pg/150 k cells) for pre-implant, 72 days post-implant, and about 90 days post-implant (post-OGTT). Furthermore, live/dead staining was conducted on fiber devices retrieved from the NSG mice at 72 and 88 days post-implant. The data is shown at FIG. 37. This data demonstrates that the kinetics of the printed fibers are similar to native tissues.

### Example 6: Printing of Annulus Fibers

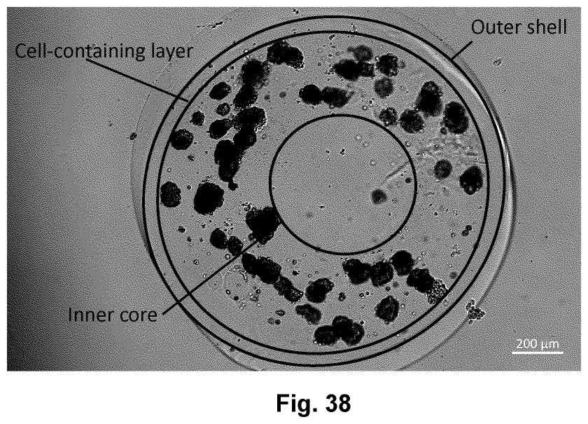

This Example demonstrates effective production of annulus fibers of the present disclosure. As illustrated at FIG. 38, a representative annulus fiber includes an inner core, a cell containing layer (i.e., annulus layer), and an outer shell. The cell layer comprises aggregates of primary human islets. Although this example depicts an annulus fiber with primary human islets, other cell types are within the scope of this disclosure as discussed herein. An annulus fiber may be printed with an RX1™ bioprinter (Aspect Biosystems, Vancouver, BC, Canada). An annulus fiber may be printed with a bioprinting system such as that disclosed in U.S. Provisional Patent Application No. 63/290,595.

Although not specifically illustrated, an annulus fiber can be segmented/compartmentalized as disclosed herein. For example, an annulus fiber can include the annulus layer (i.e., cell-containing layer), wherein the biological material (i.e., cells) is segmented/compartmentalized along the length of the fiber. There may be at least two segments of biological material, but can include many more, for example 3, 4, 5, 6, 10 or more, and so on. In brief, a method of printing such a fiber can include, via a print head having two channels leading to the annulus channel, switching flow between a cell-containing annulus layer material and a cell-free annulus layer material. The annulus layer material may be the same between the two channels, or may be different.

In the Example shown at FIG. 38, the outer shell was bioprinted, however although not specifically illustrated, in some examples an external shell may be added to a biofiber of the present disclosure by way of coating (e.g., submerging a fiber in a coating solution, dispensing a coating solution onto a bioprinted fiber, etc.). By way of example, a fiber may be coated with a pro-vasculogenic material such as polymethacrylic acid or other polymeric material.

### Example 7: Viability and Functional Studies on Cells in Annulus Fibers Compared to Core-Shell Fibers

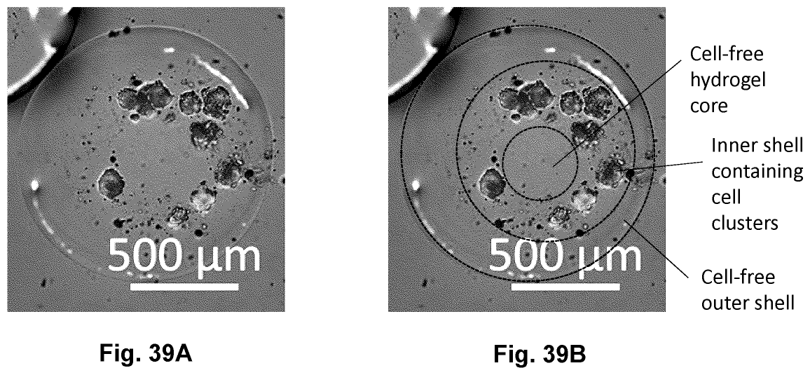

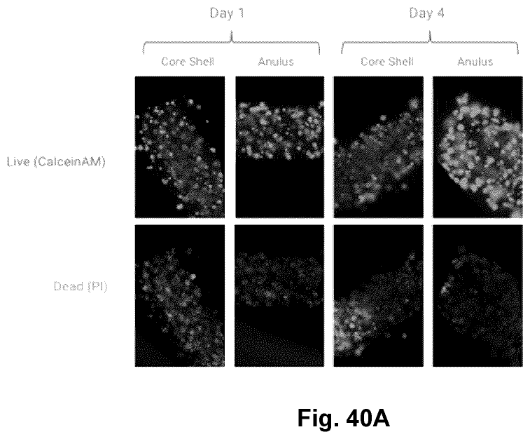

This Example demonstrates that primary hepatocyte aggregates in the first shell of an annular fiber have increased viability and functionality in in vitro culture vs primary hepatocyte aggregates encapsulated in the core of a single shell fiber of a same outer diameter. FIGS. 39A-39B depict an example image of a cross-section of a bioprinted fiber comprised of a cell-free hydrogel core, an inner shell layer containing HepG2 cell clusters, and a cell-free outer shell layer. FIG. 39B represents the same image as FIG. 39A, but with markings to clearly demarcate the different layers.

### Materials and Methods

### Live/Dead Fluorescent Staining

Calcein-AM is a cell-permeant dye that can be used to determine cell viability in most eukaryotic cells. Calcein-AM is a non-fluorescent, hydrophilic compound that easily permeates intact, live cells. In live cells Calcein-AM is converted to a hydrophilic, strongly green-fluorescent Calcein, after acetoxymethyl ester hydrolysis by intracellular esterases. Cells can be live stained and quantified without being fixed.

Propidium iodide (PI) is a red-fluorescent nucleic acid binding dye. Since PI is not permeant to live cells, it can be used to detect dead cells in a population.

Hoechst 33342 is a fluorescent nuclear stain that binds strongly to DNA. Hoechst dyes are cell-permeable and can bind to DNA in live and dead cells. Although the effectiveness of Hoechst stain is lower in live cells as the dye passes less efficiently through the membrane.

For the staining procedure, final Hoechst concentration was 20 μg/mL, final calcein-AM concentration was 5.33 μM, and final PI concentration was 15 μg/mL. Fibers were placed in a 24 well plate such that they were covered with media containing stains. One fiber was selected as a dead control, for this fiber, media was removed and 300 μL of 70% ethanol was added, and was incubated at 37° C. and 5% CO₂ for 30 in to kill the dead control. To remaining fibers, 300 μL of stain mix was added. Test fibers were incubated for 30 minutes at 37° C. and 5% CO₂ (protected from light). Tissues were imaged using a Zeiss Axio Observer Microscope (Jana, Germany). Settings for Hoechst (excitation at 385, emission at 465), settings for PI (excitation at 567, emission at 572), settings for calcein-AM, (excitation 475, emission 509).

### AlamarBlue Assay

The absorbance maximum of resazurin is 605 nm and that of resorufin is 573 nm. Either fluorescence or absorbance may be used to record results; however, fluorescence is the preferred method because it is more sensitive.

Resazurin can penetrate cells, where it becomes reduced to the fluorescent resorufin, probably as a result of the action of several different redox enzymes. The fluorescent resorufin dye can diffuse out of cells and back into the surrounding medium. Culture medium harvested from rapidly growing cells does not reduce resazurin. An analysis of the ability of various hepatic subcellular fractions suggests that resazurin can be reduced by mitochondrial, cytosolic and microsomal enzymes.

The ability of different cell types to reduce resazurin to resorufin varies depending on the metabolic capacity of the cell line and the length of incubation with the reagent. For most applications a 1- to 4-hour incubation period is adequate, however for 3D tissues 24 hrs-48 hrs is required.

AlamarBlue was added to a final concentration of 10% (440 μM stock solution). Samples were incubated for determined time period (1-4 hours for 2D cultures, 24-48 hours for 3D cultures). Media was then collected and stored at −20° C. To perform the assay, samples were removed and aliquots (50 μL) of test samples, positive, and negative controls were transferred to a 96-well flat clear bottom plate. Plates were read on a plate reader. Absorbance: 570 nm, reference 600 nm. Fluorescence (this is preferred as it is more accurate): Excitation 530-570 nm, Emission 580-620 nm). To calculate results, average relevant background control was subtracted from experimental samples and control samples. Average values were plotted for each sample. Higher concentrations of resorufin suggest more cell proliferation.

### Albumin and Urea Production Assays

Albumin (Human Albumin ELISA Kit, catalog number: ab179887; Abcam, Cambridge, UK) and Urea production assays (QuantiChrom™ Urea Assay Kit, catalog number: DIUR-100; BioAssay Systems, Hayward, CA) were conducted according to manufacturer's instructions.

### Results

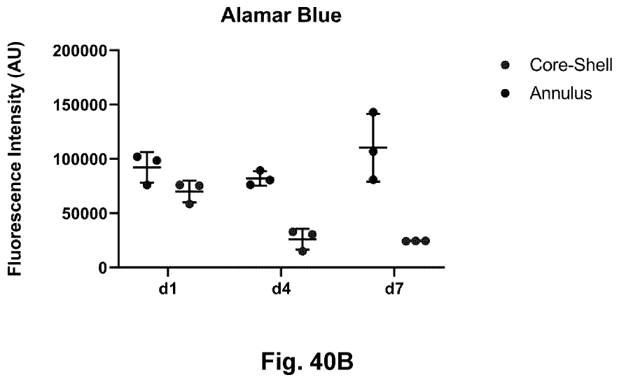

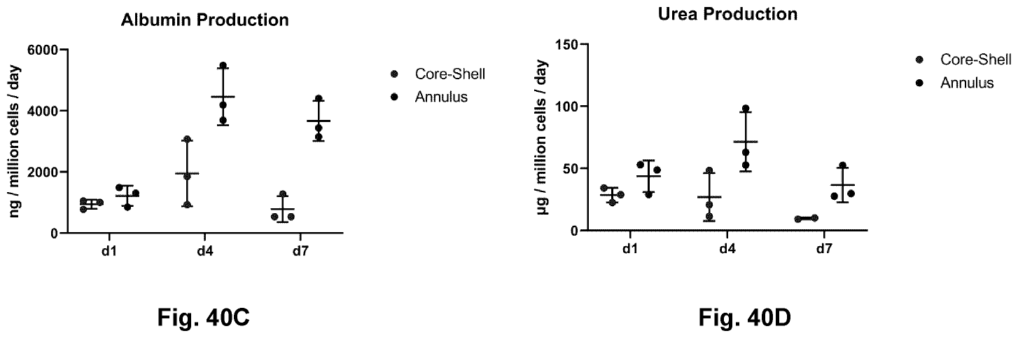

Viability of primary human hepatocyte (PHH) was shown to be improved in annulus fibers as compared to core-shell fibers. FIG. 40A depicts live (calceinAM)/dead (propidium iodide) staining of core shell and annulus fibers at day 1 and day 4 post printing. The images show lower cell viability in core shell fibers as compared to annulus fibers. Metabolic health of cells in core shell fibers and annulus fibers was quantitated using alamar Blue™ (Thermo Fisher, Waltham, MA), a non-toxic reagent that detects metabolically active cells. The data is depicted in FIG. 40B. As can be seen, metabolic health of cells in annulus fibers was greater than cells of core shell fibers.

Function of cells in core shell fibers versus annulus fibers was also examined. Albumin production was increased in annulus fibers as compared to core shell fibers (FIG. 40C), and ammonia detoxification was greater in annulus fibers as compared to core shell fibers (FIG. 40D). Taken together the data at FIGS. 39A-40D demonstrate that cell viability and function is improved in annulus fibers as compared to core shell fibers.

### Example 8: Segmentation/Compartmentalization of Core and/or Shell Materials

This Example demonstrates materials switching during the generation of core-shell fibers of the present disclosure. Core-shell fibers are used as a representative example in this particular Example, but the concept is applicable to other fibers, for example the annulus fibers of the present disclosure.

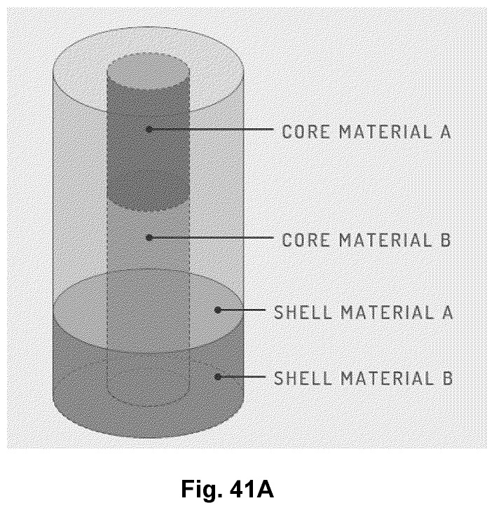

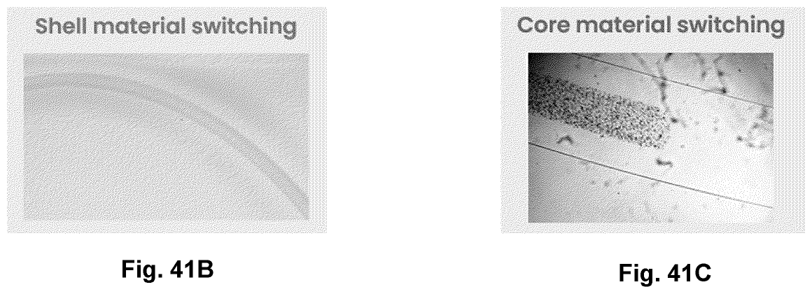

FIG. 41A depicts an exemplary illustration of a core-shell fiber of the present disclosure, where the core is comprised of a first core material (core material A) and a second core material (core material B), and wherein the shell is comprised of a first shell material (shell material A) and a second shell material (shell material B). It may be understood that FIG. 41A is meant to be illustrative, and it is within the scope of this disclosure that the core may comprise any number of different segments, and/or the outer shell may comprise any number of segments. In some embodiments, a core may comprise at least two segments, whereas the shell may be comprised of a single segment. While not explicitly illustrated, similar logic applies to annulus fibers of the present disclosure. For example, an annular fiber may include one or more of 1) a solid core having any number of segments, 2) an annulus layer having any number of segments, and 3) an external shell layer having any number of segments. In some embodiments, an annulus layer of an annulus fiber may be segmented, while an external shell and/or core may not be segmented. The annulus layer may comprise a biological material (i.e., cells).

FIG. 41B depicts an actual image of a core-shell fiber in which the external shell is comprised of two different materials. For illustrative purposes, a first visual indicator and a second visual indicator were incorporated into a first material, and a second material, respectively, so as to enable visualization of the different materials in the outer shell. As can be seen at FIG. 41B, the fiber includes two different shell materials. The process depicted at FIG. 41B can be repeated any number of times in a single fiber, depending on the application. In embodiments, one material may comprise a biological material (e.g., cells), whereas another material may be free of the biological material.

FIG. 41C depicts an actual image of a core-shell fiber in which the solid core is comprised of two different materials. Similar to that discussed with regard to FIG. 41B, the process used to make the core-shell fiber depicted at FIG. 41C may be repeated any number of times. In embodiments, one material may comprise a biological material (e.g., cells), whereas another material may be free of the biological material.

While not explicitly illustrated, the process of materials switching can be applied to both a core and an external layer (in the case of a core-shell fiber), or to a core and/or one or more layers (e.g., annulus layer and/or outer shell layer in the case of an annulus fiber).

Although the foregoing invention has been described in detail by way of illustration and example for purposes of clarity of understanding, it is readily apparent to those of ordinary skill in the art in light of the teachings of this invention that certain changes and modifications can be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

1. A bioprinted selectively permeable tissue fiber comprising a solid core, at least one annulus layer surrounding said solid core comprising at least one biological material embedded in a cross-linkable biocompatible material, and at least one external shell layer surrounding said at least one annulus layer.

2. The bioprinted selectively permeable tissue fiber according to claim 1, wherein said solid core is reinforced; preferably wherein said solid core comprises a non-biodegradable material.

3. The bioprinted selectively permeable tissue fiber according to claim 1, wherein said solid core comprises a cross-linkable material and the tissue fiber is cross-linked from the inside out.

4. The bioprinted selectively permeable tissue fiber according to claim 2, wherein said solid core comprises a non-biodegradable polymeric material selected from the group comprising or consisting of PVA.

5. The bioprinted selectively permeable tissue fiber according to claim 1 wherein said at least one biological material comprises a cell population and/or a cell-derived extracellular vesicle population in a biocompatible material.

6. The bioprinted selectively permeable tissue fiber according to claim 5, wherein the at least one biological material is homogeneously dispersed throughout the at least one annulus layer; preferably wherein the biological material is segmented/compartmentalized along the length of the fiber.

7. The bioprinted selectively permeable tissue fiber according to claim 1, wherein said biocompatible materials are selected from alginate, collagen-1, basement membrane proteins, collagen-4, collagen-2, fibronectin, vitronectin, laminin, decellularized extracellular matrices, hyaluronic acid, PEG, PEGDA and other functionalized PEG, fibrin, gelatin, GEL-MA, silk, chitosan, cellulose, POEGMA, self assembling peptides, and/or one or more functionalized alginates selected from the group consisting of methacrylated alginate, alginate furan, alginate thiol, alginate maleimide, alginate tetrazine, alginate norbornene, alginate hydrazide, DMAPS-Aid, DMAPS-Hzd, RGD-alginate, YIGSR-alginate, or combination thereof.

8. The bioprinted selectively permeable tissue fiber according to claim 5, wherein the cell population comprises cells from endocrine and exocrine glands selected from the group consisting of pancreas, liver, thyroid, pineal gland, pituitary gland, thymus, adrenal gland, ovary, testis, enteroendocrine cells, stem cells, stem-cell derived cells, or cells engineered to secrete a therapeutic agent.

9. The bioprinted selectively permeable tissue fiber according to claim 5, wherein the cell-derived extracellular vesicle population comprises exosomes.

10. The bioprinted selectively permeable tissue fiber according to claim 1, wherein said at least one external shell layer comprises immunoprotective materials and/or pro-vasculogenic materials.

11. The bioprinted selectively permeable tissue fiber according to claim 10, comprising an outer external shell layer and an inner external shell layer, wherein the outer shell layer is comprised of pro-vasculogenic materials and wherein the inner external shell layer is comprised of immunoprotective materials.

12. The bioprinted selectively permeable tissue fiber according to claim 10, wherein said immunoprotective materials are selected from alginate, chitosan, GEL-MA, PEGDA, PEG, multi-arm PEG acrylate, POEGMA, methacrylated hyaluronic acid, thiolated hyaluronic acid, PLL, hyaluronic acid, and combinations thereof.

13. The bioprinted selectively permeable tissue fiber according to claim 12, wherein said immunoprotective material comprises a functionalized alginate.

14. The bioprinted selectively permeable tissue fiber according to claim 11, wherein said immunoprotective material comprises or further comprises an acrylated zwitterionic monomer (SBMA) and PEGDA.

15. The bioprinted selectively permeable tissue fiber according to claim 10, wherein said pro-vasculogenic materials comprise one or more of fibrin, collagen, methacrylated collagen, gelatin, GEL-MA, decellularized extracellular matrix (dECM), polymethacrylic acid, RGD-alginate, YIGSR-alginate, and hyaluronic acid.

16. The bioprinted selectively permeable tissue fiber according to claim 1, wherein the diameter of said fiber is between about 2.0 mm to 0.25 mm, more preferably between about 1.5 mm to 0.45 mm, and most preferably between about 1.1 mm to 0.7 mm.

17. The bioprinted selectively permeable tissue fiber according to claim 1, wherein the diameter of said core is between about 1.92 mm to 0.05 mm, more preferably between about 1.1 mm to 0.41 mm, and most preferably between about 0.5 mm to 0.1 mm.

18. The bioprinted selectively permeable tissue fiber according to claim 1, wherein the thickness of said at least one annulus layer is between about 0.01 mm to 0.4 mm, more preferably between about 0.025 mm to 0.3 mm, and most preferably between about 0.05 mm to 0.20 mm.

19. The bioprinted selectively permeable tissue fiber according to claim 1, wherein the thickness of said at least one outer shell layer is between about 0.01 mm to 0.3 mm, more preferably between about 0.025 mm to 0.2 mm, and most preferably between about 0.05 mm to 0.2 mm.

20. A bioprinted selectively permeable tissue fiber comprising a solid core comprising at least one biological material wherein the biological material is segmented/compartmentalized along the length of the fiber, and at least one external shell layer surrounding said solid core comprising a cross-linked hydrogel material.

21. The selectively permeable bioprinted tissue fiber according to claim 20 wherein said at least one biological material comprises a cell population in a biocompatible material.

22. The bioprinted selectively permeable tissue fiber according to claim 21, wherein said biocompatible materials are selected from alginate, collagen-1, basement membrane proteins, collagen-4, collagen-2, fibronectin, vitronectin, laminin, decellularized extracellular matrices, hyaluronic acid, PEG, PEGDA and other functionalized PEG, fibrin, gelatin, GEL-MA, silk, chitosan, cellulose, POEGMA, self assembling peptides, and/or one or more functionalized alginates selected from the group consisting of methacrylated alginate, alginate furan, alginate thiol, alginate maleimide, alginate tetrazine, alginate norbornene, alginate hydrazide, DMAPS-Ald, DMAPS-Hzd, RGD-alginate, YIGSR-alginate, or combination thereof.

23. The bioprinted selectively permeable tissue fiber according to claim 20, wherein the cell population comprises cells from endocrine and exocrine glands selected from the group consisting of pancreas, liver, thyroid, parathyroid, pineal gland, pituitary gland, thymus, adrenal gland, ovary, testis, enteroendocrine cells, stem cells, stem cell-derived cells or cells engineered to secrete a therapeutic agent.

24. The bioprinted selectively permeable tissue fiber according to claim 20, wherein the cell population comprises exosomes.

25. The bioprinted selectively permeable tissue fiber according to claim 20, wherein said at least one external shell layer comprises immunoprotective materials and/or pro-vasculogenic materials.

26. The bioprinted selectively permeable tissue fiber according to claim 25, comprising an outer external shell layer and an inner external shell layer, wherein the outer shell layer is comprised of pro-vasculogenic materials and wherein the inner external shell layer is comprised of immunoprotective materials.

27. The bioprinted selectively permeable tissue fiber according to claim 25, wherein said immunoprotective materials are selected from alginate, chitosan, GEL-MA, PEGDA, PEG, multi-arm PEG acrylate, POEGMA, methacrylated hyaluronic acid, thiolated hyaluronic acid, PLL, hyaluronic acid, and combinations thereof.

28. The bioprinted selectively permeable tissue fiber according to claim 27, wherein said immunoprotective material comprises a functionalized alginate.

29. The bioprinted selectively permeable tissue fiber according to claim 26, wherein said immunoprotective material comprises or further comprises an acrylated zwitterionic monomer (SBMA) and PEGDA.

30. The bioprinted selectively permeable tissue fiber according to claim 26, wherein said pro-vasculogenic materials comprise one or more of fibrin, collagen, methacrylated collagen, gelatin, GEL-MA, decellularized extracellular matrix (dECM), polymethacrylic acid, RGD-alginate, YIGSR-alginate, and hyaluronic acid.

31. The bioprinted selectively permeable tissue fiber according to claim 20, wherein the diameter of said fiber is between about 2.0 mm to 0.2 mm, more preferably between about 1.5 mm to 0.5 mm, and most preferably between about 1.1 mm to 0.8 mm.

32. The bioprinted selectively permeable tissue fiber according to claim 20, wherein the diameter of said solid core is between about 1.98 mm to 0.18 mm more preferably between about 1.48 mm to 0.48 mm, and most preferably between about 0.9 mm to 0.50 mm.

33. The bioprinted selectively permeable tissue fiber according to claim 20, wherein the thickness of said at least one outer shell layer is between about 0.01 mm to 0.3 mm, more preferably between about 0.025 mm to 0.2 mm, and most preferably between about 0.05 mm to 0.15 mm.

34. A method for producing/secreting a biologically active agent of interest, comprising culturing bioprinted selectively permeable fibers according to claim 1 in vitro or in vivo, wherein the selectively permeable fibers comprise a biological material capable of producing/secreting the biologically active agent of interest.
